(19) United States
(12) Patent Application Publication
(10) Pub. No.: US 2007/0265223 A1
(43) Pub. Date: Nov. 15, 2007

(54) COMPOSITIONS AND METHODS OF ENHANCING SURVIVABILITY AND REDUCING INJURY OF CELLS, TISSUES, ORGANS, AND ORGANISMS UNDER HYPOXIC OR ISCHEMIC CONDITIONS

(75) Inventors: Kevin James Tomaselli, San Diego, CA (US); Thomas L. Deckwerth, Seattle, WA (US)

Correspondence Address: SEED INTELLECTUAL PROPERTY LAW GROUP PLLC, 701 FIFTH AVE, SUITE 5400, SEATTLE, WA, 98104, US

(73) Assignee: IKARIA, INC., Seattle, WA (US)

(21) Appl. No.: 11/684,968

(22) Filed: Mar. 12, 2007

Related U.S. Application Data
(60) Provisional application No. 60/783,450, filed on Mar. 17, 2006.
(60) Provisional application No. 60/781,036, filed on Mar. 10, 2006.

(51) Int. Cl. A61K 31/7076; A01N 1/02; A61P 41/00; A61P 9/10

(52) U.S. Cl. 514/46; 435/1.1

(57) ABSTRACT

The present invention provides methods, compositions, and articles of manufacture comprising adenosine, an adenosine derivative or analog, an adenosine receptor agonist, a chemical entity that is taken up by a nucleoside transporter, or a chemical entity that binds to and/or modulates a nucleoside transporter, which protect biological material from cellular or tissue damage resulting from ischemia or hypoxia, including ischemia and hypoxia due to injury, disease, or hemorrhaging. These methods, compositions, and articles of manufacture also enhance the survivability of biological material subjected to ischemia due to injury, disease, or hemorrhage.

### CROSS-REFERENCES TO RELATED APPLICATIONS

This application claims the benefit under 35 U.S.C. §119(e) of U.S. Provisional Patent Application No. 60/781,036 filed Mar. 10, 2006; and U.S. Provisional Patent Application No. 60/783,450 filed Mar. 17, 2006, where these (two) provisional applications are incorporated herein by reference in their entireties.

### BACKGROUND OF THE INVENTION

1. Field of the Invention

The present invention relates generally to the field of cell biology and physiology. More particularly, it concerns methods, compositions and articles of manufacture related to using adenosine, adenosine analogs or derivatives, adenosine receptor agonists, chemical entities that are transported into a cell by a nucleoside transporter, and chemical entities that bind and/or modulate the action of a nucleoside transporter, to enhance survivability of and/or reduce damage to cells, tissues, organs, and organisms, particularly under hypoxic or ischemic conditions.

2. Description of the Related Art

Cells, tissues, organs, and organisms that are deprived of blood flow undergo ischemic damage due to oxidative stress and eventually die. Traditional methods of reducing ischemic damage involve perfusing affected tissues with oxygen, but this procedure causes significant tissue damage and can result in serious lasting injury, such as brain damage during stroke or cardiac arrest.

Attempts have been made to reduce ischemic and reperfusion injury by inducing tissues and organs to enter a reduced metabolic state. In the context of living tissues being preserved for transplant or grafting, one common method for reducing their metabolic activity is by immersing tissues or organs in a physiologic fluid, such as saline, and placing them in the cold. However, such methods cannot be relied upon for extended periods, and the success of organ transplant and limb reattachments remains inversely related to the time the organ or limb is out of contact with the intact organism.

More extreme methods of reducing metabolic activity in whole organisms are known colloquially as “suspended animation.” Though still considered largely within the realm of science fiction, some notoriety has been achieved when wealthy individuals have sought to be cryopreserved after death in the hopes that future medical breakthroughs will permit their revival and cure of their fatal ailments. Allegedly, more than one hundred people have been cryopreserved since the first attempt in 1967, and more than one thousand people have made legal and financial arrangements for cryonics with one of several organizations, for example, Alcor Life Extension Foundation. Such methods involve the administration of anti-ischemic drugs, low temperature preservation, and methods to perfuse whole organisms with cryosuspension fluids. However, it has not yet been substantiated that this form of reduced metabolic activity is reversible.

On a related note, there are numerous reports of individuals who have survived apparent cessation of pulse and respiration after exposure to hypothermic conditions, usually in cold-water immersion. Though not fully understood by scientists, the ability to survive such situations likely derives from what is called the “mammalian diving reflex.” This reflex is believed to stimulate the vagal nervous system, which controls the lungs, heart, larynx and esophagus, in order to protect vital organs. Presumably, cold-water stimulation of nerve receptors on the skin causes shunting of blood to the brain and to the heart, and away from the skin, the gastrointestinal tract and extremities. At the same time, a protective reflex bradycardia, or slowing the heart beat, conserves the dwindling oxygen supplies within the body. Unfortunately, the expression of this reflex is not the same in all people, and is believed to be a factor in only 10-20% percent of cold-water immersion cases.

While the methods described above may be useful in certain contexts for reducing ischemic reperfusion injury, dependence upon reducing temperature can be problematic, as apparatuses and agents for producing such low temperatures may not be readily available, and damage to cells and tissue may occur as a result of freeze/thaw processes. Clearly, compositions and methods that do not rely fully on hypothermia and/or oxygen would be useful in the context of organ preservation, as well as for tissue or cell preservation. Moreover, such compositions and methods would also be useful in controlling cellular and physiologic metabolism in whole organisms subjected to traumas such as severe blood loss, hypothermia, or cardiac arrest, thereby reducing ischemic and reperfusion injury. Currently, the lack of ability to control cellular and physiologic metabolism in whole organisms subjected to such traumas is a key shortcoming in the medical field. Thus, there is a great need for improved methods for controlling metabolic processes, particularly under traumatic conditions, and pharmaceutically acceptable compositions useful in practicing these methods. The present invention fulfills these needs, and provides other related advantages.

### BRIEF SUMMARY OF THE INVENTION

The present invention is based upon the surprising discovery that adenosine can be used to enhance survivability of biological material under hypoxic or ischemic conditions, including those resulting from injury or disease. Accordingly, the present invention provides methods, composition, and articles of manufacture comprising adenosine, an adenosine derivative or analog, an adenosine receptor agonist, a chemical entity that is taken up by a nucleoside transporter, or a chemical entity that binds to and/or modulates a nucleoside transporter, for use in enhancing the survivability of and protecting a variety of different biological materials.

In one embodiment, the present invention provides a method for enhancing the survivability of a biological material exposed to ischemic or hypoxic conditions comprising contacting the biological material with an effective amount of adenosine, an adenosine derivative or analog, an adenosine receptor agonist, a chemical entity that is taken up by a nucleoside transporter, or a chemical entity that binds to and/or modulates a nucleoside transporter. In specific embodiments, the material is contacted with the adenosine, an adenosine derivative or analog, an adenosine receptor agonist, a chemical entity that is taken up by a nucleoside transporter, or a chemical entity that binds to and/or modulates a nucleoside transporter, before, during, or after being exposed to the ischemic or hypoxic conditions.

In another embodiment, the ischemic or hypoxic condition result from an injury to the material, the onset or progression of a disease that adversely affects the material, or hemorrhaging of the material. In particular embodiments, the adenosine, an adenosine derivative or analog, an adenosine receptor agonist, a chemical entity that is taken up by a nucleoside transporter, or a chemical entity that binds to and/or modulates a nucleoside transporter, is provided before the injury, before the onset or progression of the disease, or before hemorrhaging of the material. In one embodiment, the adenosine, an adenosine derivative or analog, an adenosine receptor agonist, a chemical entity that is taken up by a nucleoside transporter, or a chemical entity that binds to and/or modulates a nucleoside transporter, is not provided during or after the injury, the onset or progression of the disease, or the hemorrhaging of the material. In various embodiments, the injury is from an external physical source, including, e.g., a surgery. In one embodiment, wherein the material is provided with the adenosine, an adenosine derivative or analog, an adenosine receptor agonist, a chemical entity that is taken up by a nucleoside transporter, or a chemical entity that binds to and/or modulates a nucleoside transporter in an amount and for a time that protects the material from damage or death resulting from the injury, the onset or progression of the disease, or hemorrhaging in the material.

In various embodiments of the present invention, the biological material is selected from the group consisting of: cells, tissues, organs, organisms, and animals. In particular embodiments, it is an animal, a mammal, or a human. In other embodiments, the biological material comprises platelets. In particular embodiments, the biological material is to be transplanted or is at risk for reperfusion injury or hemorrhagic shock.

According to various embodiments of the methods of the present invention, the biological material is provided with the adenosine, an adenosine derivative or analog, an adenosine receptor agonist, a chemical entity that is taken up by a nucleoside transporter, or a chemical entity that binds to and/or modulates a nucleoside transporter, intravenously, intradermally, intraarterially, intraperitoneally, intralesionally, intracranially, intraarticularly, intraprostaticaly, intrapleurally, intratracheally, intranasally, intravitreally, intravaginally, intrarectally, topically, intratumorally, intramuscularly, intraperitoneally, intraocularly, subcutaneously, subconjunctival, intravesicularily, mucosally, intrapericardially, intraumbilically, intraocularally, orally, topically, locally, by inhalation, by injection, by infusion, by continuous infusion, by absorption, by adsorption, by immersion, by localized perfusion, via a catheter, or via a lavage. In one embodiment, 5′-AMP is provided to the biological material by infusion at a dosage in the range of 10 μg/kg/min to 350 μg/kg/min. In one embodiment, the infusion is continued for 30 minutes to 24 hours. In another embodiment the adenosine, an adenosine derivative or analog, an adenosine receptor agonist, a chemical entity that is taken up by a nucleoside transporter, or a chemical entity that binds to and/or modulates a nucleoside transporter, is provided to the material at a dosage in the range of 0.1 μmol adenosine/g material to 100 μmol adenosine/g material.

In related embodiments, the methods of the present invention further comprising exposing the biological material to a controlled pressure environment or a controlled temperature environment. In one embodiment, the controlled temperature environment is less than about 20° C. In a related embodiment, the biological material achieves a non-physiological core temperature.

In a further related embodiment, the methods of the present invention further comprise providing an effective amount of an active compound to the material. In various embodiments, the adenosine, an adenosine derivative or analog, an adenosine receptor agonist, a chemical entity that is taken up by a nucleoside transporter, or a chemical entity that binds to and/or modulates a nucleoside transporter, and the active compound are provided sequentially or are provided simultaneously.

In particular embodiments, an active compound is an oxygen antagonist, including compounds with Formula I, compounds with Formula II, or a salt or precursor thereof. In certain embodiments, the method further comprises contacting the biological material with an effective amount of an additional active compound. In more specific embodiments, the biological material is contacted with the adenosine and the additional active compound sequentially. In other more specific embodiments, the biological material is contacted with the adenosine analog and the additional active compound simultaneously. In other more specific embodiments, the additional active compound is an oxygen antagonist. In other more specific embodiments, the additional active compound is a chalcogenide compound, optionally comprising sulfur (such as H₂S) or selenium (such as H₂Se). In other more specific embodiments, the additional active compound is a chalcogenide salt, optionally comprising sulfur, selected from the group consisting of Na₂S, NaHS, K₂S, KHS, Li₂S, Rb₂S, Cs₂S, (NH₄)₂S, (NH₄)HS, BeS, MgS, CaS, SrS, and BaS, or selenium, selected from the group consisting of Na₂Se, NaHSe, K₂Se, KHSe, Li₂Se, Rb₂Se, Cs₂Se, (NH₄)₂Se, (NH₄)HSe, BeSe, MgSe, CaSe, SrSe, PoSe and BaSe. In other more specific embodiments, the additional active compound is CO₂.

In another embodiment, the active compound comprises a cationic structure capable of targeting the one or more active compounds to mitochondria.

In one embodiment, the adenosine, an adenosine derivative or analog, an adenosine receptor agonist, a chemical entity that is taken up by a nucleoside transporter, or a chemical entity that binds to and/or modulates a nucleoside transporter, is provided to the biological material as a pharmaceutical composition.

“Pharmaceutical composition” refers to a formulation of a compound and a medium generally accepted in the art for the delivery of the biologically active compound to mammals, e.g., humans. Such a medium includes all pharmaceutically acceptable carriers, diluents or excipients therefore.

“Pharmaceutically acceptable carrier, diluent or excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals.

Accordingly, the invention disclosed herein is also meant to encompass all disclosed pharmaceutically acceptable compounds being isotopically-labelled by having one or more atoms replaced by an atom having a different atomic mass or mass number. Examples of isotopes that can be incorporated into the disclosed compounds include isotopes of hydrogen, carbon, nitrogen, oxygen, fluorine, chlorine, and iodine, such as ²H, ³H, ¹¹C, ¹³C, ¹⁴C, ¹³N, ¹⁵N, ¹⁵O, ¹⁷O, ¹⁸O, ³⁵S, ¹⁸F, ³⁶Cl, ¹²³I, and ¹²⁵I, respectively. These radiolabelled compounds could be useful to help determine or measure the effectiveness of the compounds, by characterizing, for example, the site or mode of action, or binding affinity to the pharmacologically important site of action. Certain isotopically-labelled compounds, for example, those incorporating a radioactive isotope, are useful in drug and/or substrate tissue distribution studies. The radioactive isotopes tritium, i.e., ³H, and carbon-14, i.e., ¹⁴C, are particularly useful for this purpose in view of their ease of incorporation and ready means of detection. Substitution with heavier isotopes such as deuterium, i.e., ²H, may afford certain therapeutic advantages resulting from greater metabolic stability, for example, increased in vivo half-life or reduced dosage requirements, and hence may be preferred in some circumstances. Substitution with positron emitting isotopes, such as ¹¹C, ¹⁸F, ¹⁵O and ¹³N, can be useful in Positron Emission Topography (PET) studies for examining substrate receptor occupancy. Isotopically-labeled compounds can generally be prepared by conventional techniques known to those skilled in the art or by processes analogous to those described in the Preparations and Examples as set out below using an appropriate isotopically-labeled reagent in place of the non-labeled reagent previously employed.

“Therapeutically effective amount” refers to that amount of a compound of the invention which, when administered to a mammal, preferably a human, is sufficient to effect treatment, as defined below, of a disease or condition in the mammal, preferably a human. The amount of a compound of the invention which constitutes a “therapeutically effective amount” will vary depending on the compound, the condition and its severity, the manner of administration, and the age of the mammal to be treated, but can be determined routinely by one of ordinary skill in the art having regard to his own knowledge and to this disclosure.

“Treating” or “treatment” as used herein covers the treatment of the disease or condition of interest in a mammal, preferably a human, having the disease or condition of interest, and includes: (i) preventing the disease or condition from occurring in a mammal, in particular, when such mammal is predisposed to the condition but has not yet been diagnosed as having it; (ii) inhibiting the disease or condition, i.e., arresting its development; (iii) relieving the disease or condition, i.e., causing regression of the disease or condition; or (iv) relieving the symptoms resulting from the disease or condition. As used herein, the terms “disease” and “condition” may be used interchangeably or may be different in that the particular malady or condition may not have a known causative agent (so that etiology has not yet been worked out) and it is therefore not yet recognized as a disease but only as an undesirable condition or syndrome, wherein a more or less specific set of symptoms have been identified by clinicians.

In yet another related embodiment, the present invention includes a method for preventing or reducing damage to a biological material exposed to ischemic or hypoxic conditions comprising providing to the biological material an effective amount of adenosine, an adenosine derivative or analog, an adenosine receptor agonist, a chemical entity that is taken up by a nucleoside transporter, or a chemical entity that binds to and/or modulates a nucleoside transporter.

In another embodiment, the present invention includes a method for reversibly inhibiting metabolism in a biological material comprising providing to the biological material an effective amount of adenosine, an adenosine derivative or analog, an adenosine receptor agonist, a chemical entity that is taken up by a nucleoside transporter, or a chemical entity that binds to and/or modulates a nucleoside transporter. In one embodiment, the biological material is a mammal.

In a further embodiment, the present invention includes a method of enhancing survivability of a mammal to hemorrhagic shock, comprising providing to the organism an effective amount of adenosine, an adenosine derivative or analog, an adenosine receptor agonist, a chemical entity that is taken up by a nucleoside transporter, or a chemical entity that binds to and/or modulates a nucleoside transporter. In a particular embodiment, the mammal suffers hemorrhagic shock after being provided with the adenosine or related compound.

In another embodiment, the present invention includes a method of enhancing survivability of a mammal undergoing a surgery, comprising providing to the mammal an effective amount of adenosine, an adenosine derivative or analog, an adenosine receptor agonist, a chemical entity that is taken up by a nucleoside transporter, or a chemical entity that binds to and/or modulates a nucleoside transporter, prior to the surgery. In particular embodiments, the surgery is selected from elective surgery, planned surgery, or emergency surgery.

In a further embodiment, the present invention includes a method of preserving biological material ex vivo comprising contacting the biological material with adenosine, an adenosine derivative or analog, an adenosine receptor agonist, a chemical entity that is taken up by a nucleoside transporter, or a chemical entity that binds to and/or modulates a nucleoside transporter. In one embodiment, the biological material is stored at low temperature. In particular embodiments, the biological material is cells, tissue, or an organ. In one embodiment, the cells are platelets. In one embodiment, the tissue or organ is being stored prior to transplant.

In another related embodiment, the present invention provides a method of preserving non-living biological material, comprising contacting said material with adenosine, an adenosine derivative or analog, an adenosine receptor agonist, a chemical entity that is taken up by a nucleoside transporter, or a chemical entity that binds to and/or modulates a nucleoside transporter, and/or an active compound. In one embodiment, the non-living biological material is a dead animal. In another embodiment, the non-living biological material is a plant or plant product. In a particular embodiment, the plant or plant product is a food product.

In yet another related embodiment, the present invention provides a method of extending the shelf-life of a food or beverage product subject to spoilage, comprising contacting the food or beverage product with adenosine, an adenosine derivative or analog, an adenosine receptor agonist, a chemical entity that is taken up by a nucleoside transporter, or a chemical entity that binds to and/or modulates a nucleoside transporter and/or an active compound. In one embodiment, the food or beverage product is wine or beer. In various embodiments, the product comprises adenosine, an adenosine derivative or analog, an adenosine receptor agonist, a chemical entity that is taken up by a nucleoside transporter, or a chemical entity that binds to and/or modulates a nucleoside transporter and/or an active compound.

In another embodiment, the present invention includes a method of extending the shelf-life of a commercial product comprising contacting the commercial product with adenosine, an adenosine derivative or analog, an adenosine receptor agonist, a chemical entity that is taken up by a nucleoside transporter, or a chemical entity that binds to and/or modulates a nucleoside transporter and/or an active compound. In one embodiment, the commercial product is a pharmaceutical, health care, or cosmetic product. In various embodiments, the product comprises an active compound.

In another embodiment, the present invention includes a pharmaceutical composition comprising adenosine, an adenosine derivative or analog, an adenosine receptor agonist, a chemical entity that is taken up by a nucleoside transporter, or a chemical entity that binds to and/or modulates a nucleoside transporter, as well as an active compound and a pharmaceutically acceptable diluent or carrier, wherein said composition is formulated for administration to a mammal.

In another embodiment, the present invention includes an article of manufacture comprising packaging material and, contained within the packaging material, adenosine, an adenosine derivative or analog, an adenosine receptor agonist, a chemical entity that is taken up by a nucleoside transporter, or a chemical entity that binds to and/or modulates a nucleoside transporter, wherein the packaging material comprises a label that indicates that the adenosine, an adenosine derivative or analog, an adenosine receptor agonist, a chemical entity that is taken up by a nucleoside transporter, or a chemical entity that binds to and/or modulates a nucleoside transporter can be used to enhance the survivability of biological material exposed to ischemic or hypoxic conditions. In one embodiment, the adenosine is 5′-AMP. In another embodiment, the article further comprises a pharmaceutically acceptable diluent. In one embodiment, the adenosine, an adenosine derivative or analog, an adenosine receptor agonist, a chemical entity that is taken up by a nucleoside transporter, or a chemical entity that binds to and/or modulates a nucleoside transporter is provided in a first sealed contained and the pharmaceutically acceptable diluent is provided in a second sealed container. In another embodiment, the article further comprises an active compound. In a related embodiment, the adenosine, an adenosine derivative or analog, an adenosine receptor agonist, a chemical entity that is taken up by a nucleoside transporter, or a chemical entity that binds to and/or modulates a nucleoside transporter is provided in a first sealed container and the active compound is provided is a second sealed container.

In various embodiments of methods, compositions and articles of the present invention, the adenosine is adenosine monophosphate (AMP). In particular embodiments, the AMP is 5′-AMP.

### BRIEF DESCRIPTION OF THE SEVERAL VIEWS OF THE DRAWING(S)

FIGS. 1A and 1B provide graphs depicting the protective effect of graduated adenosine 5′ monophosphate (5′-AMP) treatment on mice subjected to lethal hypoxia. These graphs plot the subcutaneous body temperature of the mice over time, and indicate the starting points and dosages of 5′AMP and hypoxia treatments. FIG. 1A provides the results obtained from a mouse (MJVC80) treated with 5′-AMP, and FIG. 1B provides the results obtained from an untreated mouse (MJVC81).

FIGS. 2A and 2B provide graphs depicting the protective effect of constant 5′-AMP treatment on mice subjected to lethal hypoxia. These graphs plot the subcutaneous body temperature of the mice over time, and indicate the starting points and dosages of 5′AMP and hypoxia treatments. FIG. 2A provides the results obtained from a mouse (MJVC83) treated with 5′-AMP, and FIG. 2B provides the results obtained from an untreated mouse (MJVC84).

FIGS. 3A and 3B provide graphs depicting the protective effect of 5′-AMP bolus treatment followed by constant treatment on rats subjected to lethal hypoxia. These graphs plot the subcutaneous body temperature of the rats over time, and indicate the starting points and dosages of 5′AMP and hypoxia treatments. FIG. 3A provides the results obtained from a rat (RJVC35) treated with 5′-AMP, and FIG. 3B provides the results obtained from an untreated rat (RJVC36).

FIGS. 4A, 4B, 5A, and 5B provide graphs depicting the protective effect adenosine pre-treatment on mice subjected to lethal hypoxia. These graphs plot the subcutaneous body temperature of the mice over time, and indicate the starting points and dosages of adenosine and hypoxia treatments. FIGS. 4A and 5A provides the results obtained from mice (MJVC104 and MJVC107) treated with adenosine, and FIGS. 4B and 5B provides the results obtained from control mice (MJVC105 and MJVC108) treated with sodium chloride.

FIG. 6 provides chemical structures of selected adenosine receptor agonists acting at the A₁ subtype.

FIG. 7 provides chemical structures of selected N⁶-substituted adenosine derivatives that are selective A₁ agonists.

FIG. 8 provides chemical structures of selected A₁ receptor agonists that provide high neuroprotective and low cardiovascular effects.

FIG. 9 provides chemical structures of selected adenosine A₁ receptor agonists that are highly selective versus adenosine A_{2A} and A₃ receptors.

FIG. 10 provides chemical structures of selected 8-alkylamino derivatives of N⁶-cyclopentyladenosine (CPA), which are selective partial agonists for adenosine A₁ receptors.

FIG. 11 provides chemical structures of selected A₁ selective partial agonists characterized by high receptor affinity.

FIGS. 12A and 12B provide chemical structures of selected adenosine receptor agonists. FIG. 12A shows adenosine receptor agonists acting at the A_{2A} receptor subtype. FIG. 12B shows agonists selective for the A₃ receptor subtype.

FIG. 13 provides chemical structures for CVT-510, an A₁ adenosine receptor agonist, and a number of CVT-510 analogs with C5′ modifications and incorporated C8-substituents.

FIG. 14 provides the generic chemical structure of 5′-deoxy-5′-fluoro substituted adenosines.

FIG. 15 provides chemical structures of selected partial agonists at A₁ adenosine receptors.

FIG. 16 provides chemical structures of selected A_{2A} agonists with unaltered ribose moieties.

FIG. 17 provides chemical structures of selected A_{2A} agonists with modified ribose moieties.

FIG. 18 provides chemical structures of selected A_{2A} agonists.

FIG. 19 provides chemical structures of selected A_{2A} agonists.

FIGS. 20A and 20B provide chemical structures of selected adenosine receptor agonists. FIG. 20A shows A_{2B} adenosine receptor agonists. FIG. 20B shows non-nucleoside A₁, A_{2A}, and A_{2B} agonists.

FIG. 21 provides chemical structures of selected 3′-deoxy-3′-amino-substituted adenosine analogs with high A₃ adenosine receptor affinity.

FIG. 22 provides chemical structures of selected partial A₃ agonists.

FIG. 23 provides chemical structures of selected nonselective and A₃-selective adenosine receptor agonists.

FIG. 24 provides chemical structures of selected allosteric enhancers of the activity of adenosine receptor agonists. PD81,723 and T-62 enhance the activity of agonists acting at the A₁ adenosine receptor, and VUF5455 and DU124183 enhance the activity of agonists acting at the A₃ adenosine receptor.

FIG. 25 provides chemical structures of selected carbocyclic adenosine analogs.

FIG. 26 provides chemical structures of 2-amino-benzoylthiopene and derivatives able to selectively enhance the agonist binding to the adenosine A₁ receptor.

FIG. 27 provides chemical structures of selected 2-(Ar)alkynyl-substituted adenosine derivatives.

FIG. 28 provides chemical structures of selected dimeric A₁-A₃-adenosine receptor agonist conjugates.

FIG. 29 provides chemical structures of selected nucleosides and nucleoside analogs.

### DETAILED DESCRIPTION OF THE INVENTION

The present invention provides methods, compositions, and articles of manufacture that protect biological material from cellular or tissue damage resulting from ischemia or hypoxia due to injury, disease, or hemorrhaging. These methods, compositions, and articles of manufacture also enhance the survivability of biological material subjected to ischemia due to injury, disease, or hemorrhage.

The present invention is based, in large part, on the surprising discovery that adenosine, including adenosine 5′ monophosphate (5′AMP), enhances the survivability of organisms exposed to hypoxic conditions. This discovery further establishes that adenosine protects biological material from damage resulting from ischemic or hypoxic conditions, including those resulting from injury or disease. In addition, this finding supports the use of adenosine derivatives and analogs, as well as adenosine receptor agonists, chemical entities that bind to and/or are taken up by nucleoside transporters, to enhance survivability and/or protect biological material from injury due to ischemic or hypoxic conditions.

A previous study to identify metabolic signals regulating circadian rhythms in mammals reported that 5′-AMP levels were elevated in animals in a state of torpor induced by metabolic stress (Zhang et al., Nature 439(19):340-343, 2006). In addition, treatment of animals with 5′-AMP induced torpor, as endured by depressed body temperature and induced expression of the mClps gene. These studies implicated 5′-AMP as a circadian signal that regulates mClps expression and, thereby, induces torpor. Given the role of mClps in satiety reduction and the activation of fat catabolism, the authors of this study postulated that 5′-AMP and analogs thereof could be used to treat human obesity and insulin-resistant type-2 diabetes, and further suggested that the ability of 5′-AMP to induce torpor could be a tool in core body temperature (CBT) management during major surgery or emergency room trauma response. A likely mechanism for the effects of 5′-AMP observed by Zheng et al. involve the metabolism or conversion of 5′-AMP to adenosine and the resulting interaction of adenosine with either adenosine receptors, nucleotide transporters, or both, in cells that control body temperature and/or are capable of expressing the mClps gene in response to adenosine.

As described in the accompanying Examples, the present invention is the first demonstration that treatment with adenosine, or 5′-AMP, enhances the survivability of animals subjected to hypoxic conditions. This discovery provides the scientific basis for the use of adenosine and related chemical entities described herein, including 5′AMP, to also prevent cellular and tissue damage associated with hypoxic conditions, including those caused by injury or disease.

In particular embodiments, methods and compositions of the present invention are used to enhance survivability of or prevent injury to biological material after an injury (e.g., an accidental injury) or after the onset or progression of a disease, in order to protect the biological matter from damage associated with the injury or disease. In such embodiments, the biological material is contacted with adenosine, an adenosine derivative or analog, an adenosine receptor agonist, a chemical entity that is taken up by a nucleoside transporter, or a chemical entity that binds to and/or modulates a nucleoside transporter, after the injury or the onset or progression of a disease.

In other embodiments, methods of the present invention are used to enhance survivability of or prevent injury to biological matter prior to an injury (e.g., an elective surgery) or prior to the onset or progression of a disease, in order to protect the biological matter from damage associated with the injury or disease. In such embodiments, the biological material is contacted with adenosine, an adenosine derivative or analog, an adenosine receptor agonist, a chemical entity that is taken up by a nucleoside transporter, or a chemical entity that binds to and/or modulates a nucleoside transporter, before the injury or the onset or progression of a disease. Such methods are generally referred to as “pre-treatment.” In various embodiments, pretreatment includes methods wherein biological material is contacted either before, both before and during, or before, during, and after biological matter is subjected to adverse conditions (e.g., an injury or onset or progression of a disease).

In certain embodiments, the present compositions and methods of the present invention induce biological material to enter a hypometabolic state wherein the biological material is alive but is characterized by one or more of: (1) at least a 50% reduction in the rate or amount of carbon dioxide production by the biological matter; and (2) at least a 50% reduction in the rate or amount of oxygen consumption by the biological matter. In another embodiment, the compositions and methods of the present invention induce biological material to enter a hypometabolic state wherein the biological material is alive but is characterized by one or more of: (1) a less than 50% reduction in the rate or amount of carbon dioxide production by the biological matter; and (2) a less than 50% reduction in the rate or amount of oxygen consumption by the biological matter. Any assay to measure oxygen consumption or carbon dioxide production may be employed, and a typical assay will involve utilizing a closed environment and measuring the difference between the oxygen put into the environment and oxygen that is left in the environment after a period of time. Typically, any reduction in the metabolic activity of a biological material is reversible.

According to various embodiments of the methods of the present invention, a hypometabolic state is induced by treating biological material with an amount of adenosine, an adenosine derivative or analog, an adenosine receptor agonist, a chemical entity that is taken up by a nucleoside transporter, or a chemical entity that binds to and/or modulates a nucleoside transporter, that induces hypometabolism directly itself or, alternatively, by treating biological material with an amount of adenosine that does not itself induce hypometabolism, but instead, promotes or enhances the ability of or decreases the time required for the biological material to enter a hypometabolic state in response to another stimuli, such as, but not limited to, an injury, a disease, hypoxia, reduced temperature conditions, excessive bleeding, or treatment with one or more other active compounds (as defined herein).

It will be appreciated that the length of time with which biological material is contacted with adenosine, an adenosine derivative or analog, an adenosine receptor agonist, a chemical entity that is taken up by a nucleoside transporter, or a chemical entity that binds to and/or modulates a nucleoside transporter, and the amount of adenosine, an adenosine derivative or analog, an adenosine receptor agonist, a chemical entity that is taken up by a nucleoside transporter, or a chemical entity that binds to and/or modulates a nucleoside transporter, used will vary depending upon the type of biological material, the desired outcome, the particular type of injury or disease, and the particular type of ischemic challenge faced by the biological material. For example, inducing a hypometabolic state with respect to a whole animal and with respect to cells or tissues may require different lengths of treatment with adenosine. In addition, with respect to human subjects, e.g., subjects undergoing a surgical treatment, treatment for a hemorrhagic shock, or treatment for a hyperproliferative disorder, maintaining the subject in a hypometabolic state for 12, 18, or 24 hours is generally contemplated. With respect to non-human animal subjects, e.g. non-human animals shipped or stored for commercial purposes, maintaining the subject in a hypometabolic state for a period of 1 or 2 days, 3 or 4 days, 5 or 7 days, 1 or 2 weeks, 2 or 4 weeks, or longer is contemplated.

In addition, it is understood that the amount of adenosine, an adenosine derivative or analog, an adenosine receptor agonist, a chemical entity that is taken up by a nucleoside transporter, or a chemical entity that binds to and/or modulates a nucleoside transporter, required will vary depending upon whether the biological material is also being treated with another stimuli, i.e., an agent or conditions that induces a hypometabolic state. In such circumstances, the biological material may be contacted with adenosine, an adenosine derivative or analog, an adenosine receptor agonist, a chemical entity that is taken up by a nucleoside transporter, or a chemical entity that binds to and/or modulates a nucleoside transporter, for all or only a part of the duration of time the method is performed, in order to, e.g., enhance survivability of the biological material or protect it from ischemic damage.

Furthermore, it is understood that in all embodiments of methods of the present invention, biological matter may be contacted with one or two or more chemical entities selected from: adenosine, adenosine analogs and derivatives, adenosine receptor agonists, chemical entities that are transported into a cell by a nucleoside transporter, and chemical entities that bind and/or modulate the action of a nucleoside transporter. Similarly, compositions and articles of manufacture of the present invention may comprise, in various embodiments, one or two or more chemical entities selected from: adenosine, adenosine analogs and derivatives, adenosine receptor agonists, chemical entities that are transported into a cell by a nucleoside transporter, and chemical entities that bind and/or modulate the action of a nucleoside transporter. In various embodiments, contact may occur simultaneously, during overlapping time period, or at different times.

The term “biological material” refers to any living biological material, including cells, tissues, organs, and/or organisms, and any combination thereof. It is contemplated that the methods of the present invention may be practiced on a part of an organism (such as in cells, in tissue, and/or in one or more organs), whether that part remains within the organism or is removed from the organism, or on the whole organism. Moreover, it is contemplated in the context of cells and tissues, both homogenous and heterogeneous cell populations may be the subject of embodiments of the invention. The term “in vivo biological matter” refers to biological matter that is in vivo, i.e., still within or attached to an organism. Moreover, the term “biological matter” will be understood as synonymous with the term “biological material.” In certain embodiments, it is contemplated that one or more cells, tissues, or organs is separate from an organism. The terms “isolated” and “ex vivo” are used to describe such biological material. It is contemplated that the methods of the present invention may be practiced on in vivo and/or isolated biological material.

The cell treated according to the methods of the present invention may be eukaryotic or prokaryotic. In certain embodiments, the cell is eukaryotic. More particularly, in some embodiments, the cell is a mammalian cell. Mammalian cells include, but are not limited to those from a: human, monkey, mouse, rat, rabbit, hamster, goat, pig, dog, cat, ferret, cow, sheep, and horse.

Cells of the invention may be diploid but in some cases, the cells are haploid (sex cells). Additionally, cells may be polyploid, aneuploid, or anucleate. In particular embodiments, a cell is from a particular tissue or organ, such as one from the group consisting of: heart, lung, kidney, liver, bone marrow, pancreas, skin, bone, vein, artery, cornea, blood, small intestine, large intestine, brain, spinal cord, smooth muscle, skelet al muscle, ovary, testis, uterus, and umbilical cord. In certain embodiments, cell are characterized as one of the following cell types: platelet, myelocyte, erythrocyte, lymphocyte, adipocyte, fibroblast, epithelial cell, endothelial cell, smooth muscle cell, skelet al muscle cell, endocrine cell, glial cell, neuron, secretory cell, barrier function cell, contractile cell, absorptive cell, mucosal cell, limbus cell (from cornea), stem cell (totipotent, pluripotent or multipotent), unfertilized or fertilized oocyte, or sperm.

The terms “tissue” and “organ” are used according to their ordinary and plain meanings. Though tissue is composed of cells, it will be understood that the term “tissue” refers to an aggregate of similar cells forming a definite kind of structural material. Moreover, an organ is a particular type of tissue. In certain embodiments, the tissue or organ is “isolated,” meaning that it is not located within an organism.

In various embodiments, methods of the present invention are used to treat any type of organism, including but not limited to, mammals, reptiles, amphibians, birds, fish, invertebrates, fungi, plants, protests, and prokaryotes. In particular embodiments, a mammal is a marsupial, an insect, a primate, or a rodent. In other embodiments, an organism is a human or a non-human animal. In specific embodiments, an animal is a mouse, rat, cat, dog, horse, cow, rabbit, sheep, fruit fly, frog, worm, or human.

1. Adenosine and Related Chemical Entities

Various embodiments of the compositions, methods, and articles of manufacture of the present invention include adenosine, an adenosine derivative or analog, an adenosine receptor agonist, a chemical entity that is taken up by a nucleoside transporter, or a chemical entity that binds to and/or modulates a nucleoside transporter. Collectively, adenosine and these related chemical entities are referred to as “adenosine and related chemical entities.” It is understood that while specific illustrative embodiments described herein may refer to adenosine, these embodiments may also be practiced using adenosine derivatives and analogs, adenosine receptor agonists, and chemical entities that bind to and/or is taken up by nucleoside transporters. Furthermore, in various embodiments, chemical entities used according to the present invention are organic compounds, e.g., small molecules, inorganic compounds, polypeptides, e.g., antibodies, or nucleic acids.

In certain embodiments, compositions and methods of the present invention are practiced using adenosine and various salts, derivatives, and analogs thereof. The present invention contemplates the use of any form of adenosine, including but not limited to those described herein.

Adenosine is a nucleoside comprised of adenosine attached to a ribose (ribofuranose) moiety via a β-N₉-glycosidic bond. Adenosine plays an important role in biochemical processes, such as energy transfer, as adenosine triphosphate (ATP) and adenosine diphosphate (ADP), as well as in signal transduction as cyclic adenosine monophosphate (cAMP). If attached to a deoxyribose ring, it is referred to as deoxyadenosine adenosine.

In one embodiment, the adenosine is adenosine monophosphate (AMP). AMP is an organic compound composed of an adenosine base, the sugar ribose, and one phosphate unit. AMP is one of the possible products of the hydrolysis of adenosine triphosphate (ATP) and is, therefore, important in the transfer of chemical energy during anabolism. In particular embodiments, the methods are practiced using 5′-AMP, which includes synthetic 5′-AMP.

In related embodiments, compositions and methods of the present invention are practiced using an adenosine derivative, analog or mimetic, such as a 5′-AMP analog. Examples of adenosine analogs are described, e.g., in U.S. Pat. Nos. 6,174,873, 5,968,911, 5,491,146, 5,998,386, 5,998,387, 6,043,224, and 6,329,349. These include, but are not limited to: acid-labile s′-deoxyadenosine analogs; 2′-deoxycoformycin (also referred to as dCF, pentostatin, or NIPENT.RTM.), an inhibitor of adenosine deaminase; fludarabine monophosphate (FLU), a fluorinated analogue of adenine that is relatively resistant to adenosine-deaminase; and 2-chloro-2′-deoxyadenosine (also known as cladribine or 2CDA), a drug also resistant to adenosine deaminase through introduction of a chlorine at the 2 carbon. Another example is 2-chloro-N⁶-cyclopentyl-adenosine (CCPA). Other adenosine analogs that have useful activity include deoxyadenosines, generally, including 2′-deoxyadenosine, 3′-deoxyadenosine, and dideoxyadenosine. In another embodiment, the analog is AmP579, a mixed adenosine agonist with both A1 and A2 effects (Rhone-Poulenc Rorer, Collegeville, Pa.). The analog may also be DPMA, a selective adenosine A₂ receptor agonist, CPA, a selective adenosine A₁ receptor agonist, Benzyl-NECA, a selective A₃ receptor agonist, 2-chloroadenosine, a non selective A₂/A₁ agonist, or NECA, a non-selective A₂/A₁ agonist.

A variety of other adenosine analogs are commercially available, including, e.g., 3′-Amino-3′-deoxyadenosine (3′-AdA) 5′-Amino-5′-deoxyadenosine (5′-NH₂-Ado), 3′-Amino-3′-deoxyguanosine (3′-AdG) 8-Azidoadenosine (8-N₃-Ado), 8-Bromo-2′-deoxyadenosine (8-Br-dAdo), 8-Bromo-2′-deoxyguanosine (8-Br-dGuo), N⁶-Carbamoylthreonyladenosine (t6-Ado), 2-Chloroadenosine (2-Cl-Ado/2-CADO), 8-Chloroadenosine (8-Cl-Ado), 2′-O-Methyladenosine-3′,5′-cyclic monosphosphate (2′-O-Me-cAMP), Adenosine-3′,5′-cyclic monophosphorothioate, Rp-isomer ( Rp-cAMPS), Adenosine-3′,5′-cyclic monophosphorothioate, Rp-isomer ( Rp-cAMPS), 8-Bromoadenosine-3′,5′-cyclic monophosphorothioate, Rp-isomer ( Rp-8-Br-cAMPS), 8-Bromo-2′-monobutyryladenosine-3′,5′-cyclic monophosphorothioate, Rp-isomer ( Rp-8-Br-MB-cAMPS), 8-Chloroadenosine-3′,5′-cyclic monophosphorothioate, Rp-isomer ( Rp-8-Cl-cAMPS), 8-(4-Chlorophenylthio)adenosine-3′,5′-cyclic monophosphorothioate, Rp-isomer ( Rp-8-CPT-cAMPS), 8-Hexylaminoadenosine-3′,5′-monophosphorothioate, Rp-isomer (Rp-8-HA-cAMPS), 8-Hydroxyadenosine-3′,5′-monophosphorothioate, Rp-isomer (Rp-8-OH-cAMPS), 2′-0-Monobutyryladenosine-3′,5′-cyclic monophosphorothioate, Rp-isomer (Rp-2′-O-MB-cAMPS), 8-Piperidinoadenosine-3′,5′-cyclic monophosphorothioate, Rp-isomer (Rp-8-PIP-cAMPS), 8-(2-Aminoethylamino)adenosine-3′,5′-monophosphorothioate, Rp-isomer (Rp-8-AEA-cAMPS) , 8-(6-Aminohexylamino)adenosine-3′,5′-monophosphorothioate, Rp-isomer (Rp-8-AHA-cAMPS), 2′-(6-Aminohexylcarbamoyl)adenosine-3′,5′-monophosphorothioate, Rp-isomer (Rp-2′-AHC-cAMPS) , 8-Azidoadenosine-3′,5′-monophosphorothioate, Rp-isomer (Rp-8-N₃-cAMPS), 2-Chloroadenosine-3′,5′-monophosphorothioate, Rp-isomer (Rp-2-Cl-cAMPS) , 6-Chloropurine riboside-3′,5′-monophosphorothioate, Rp-isomer (Rp-6-Cl-cPuMPS) , 7-Deazaadenosine-3′,5′-monophosphorothioate, Rp-isomer (Rp-7-CH-cAMPS), 6-Dimethylaminopurine riboside-3′,5′-monophosphorothioate, Rp-isomer (Rp-6-DMA-cAMPS) , 6-Ethylthiopurine riboside-3′,5′-monophosphorothioate, Rp-isomer (Rp-6-EtS-cAMPS) , Inosine-3′,5′-monophosphorothioate, Rp-isomer (Rp-cIMPS) , 8-lodoadenosine-3′,5′-monophosphorothioate, Rp-isomer (Rp-8-I-cAMPS) , N⁶-Phenyladenosine-3′,5′-monophosphorothioate, Rp-isomer (Rp-6-Phe-cAMPS), and Purine riboside-3′, 5′-monophosphorothioate, Rp-isomer (Rp-cPuMPS) (Biolog Life Science Institute, Bremen, Germany).

Additional examples of adenosine analogs useful according to various embodiments of the present invention include, but are not limited to, 3-deaza-adenosine (DZA), 3-deaza-(±)aristeromycin (DZAri), 2′,3′-dideoxy-adenosine (ddAdo), 2′,3′-dideoxy-3-deaza-adenosine (ddDZA), 2′,3′-dideoxy-3-deaza-(±)aristeromycin (ddDZAri), 3-deaza-5′-(±)noraristeromycin (DZNAri), 3-deaza-neplanocin A (DZNep), and neplanocin A (NepA), as well as the acyclic adenosine analogs, (S)-9-(3-hydroxy-2-phosphonylmethoxypropyl)adenine [(S)-HPMPA], 9-(2-phosphonylmethoxyethyl)adenine (PMEA), and (S)-9-(2,3-dihydroxypropyl)adenine [(S)-DHPA] (Lin et al.).

In one particular embodiment, the adenosine analog is adenosine-5′-O-monophosphorothioate or a salt thereof, e.g., sodium salt (Axxora Platform, San Diego, Calif.). In another, it is adenosine 5′-monophosphoramidate (Sigma-Aldrich, St. Louis, Mo.).

In certain embodiments, a 5′-AMP analog is 5-aminoimidazole-4-carboxamide ribonucleoside (AICAR), shown in Formula III, or a derivative or analog thereof. 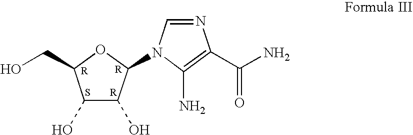
Examples of AICAR derivatives or analogs include, but are not limited to: 1H-imidazole-4-carboxamide, 5-amino-1-(5-O-phosphono-β-D-ribofuranosyl), shown in Formula IV; L-Aspartic acid, N-[[5-amino-1-(5-O-phosphono-β-D-ribofuranosyl)-1H-imidazol-4-yl]carbonyl], shown in Formula V; 1H-Imidazole-4-carboxylic acid, 5-amino-1-(3,5-O-phospinico-β-D-ribofuranosyl) (cyclic AICAR-3′,5′-MP), shown in Formula VI; 1H-Imidazole-4-carboxylic acid, 5-amino-1-(3,5-O-phosphinico-b-D-ribofuranosyl), 4-ethyl ester (cyclic carboxyethyl-AICAR-3′,5′-MP), shown in Formula VII, 5-aminoimidazole-4-carboxamide-1-b-D-ribofuranosyl 5′-monophosphate (AICAR 5′-monophosphate), and AICAR formyltransferase.

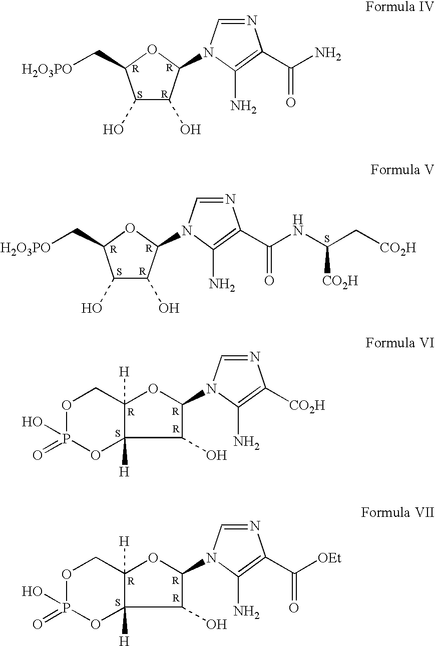

In certain embodiments, compositions and methods of the present invention are practiced using a chemical entity that binds to and/or activates adenosine receptors (e.g., an adenosine receptor agonist). Such chemical entities are known in the art and include chemical entities that agonize any, two or more, or all of the adenosine receptor subtypes, including A₁, A₂ₐ, A_{2b}, and A₃ adenosine receptors. Various adenosine receptor subtypes and agonists are described in the scientific literature, including, e.g., Muller CE, “Medicinal chemistry of adenosine A3 receptor ligands,” Curr Top Med Chem. 3(4):445-62, 2003; Cristalli G et al., “Medicinal chemistry of adenosine A2A receptor agonists,” Curr Top Med Chem. 3(4):387-401, 2003; Gao ZG, et al., “Partial agonists for A(3) adenosine receptors,” Curr Top Med Chem. 4(8):855-62, 2004; Zablocki JA et al., “Partial A(1) adenosine receptor agonists from a molecular perspective and their potential use as chronic ventricular rate control agents during atrial fibrillation (AF),” Curr Top Med Chem. 4(8):839-54, 2004; Dalpiaz A et al., “Adenosine A(1) receptor: analysis of the potential therapeutic effects obtained by its activation in the central nervous system,” Curr Med Chem. 9(21):1923-37, 2002 Nov; Cristalli G et al., “Medicinal chemistry of adenosine A2A receptor agonists,” Curr Top Med Chem. 3(4):387-401, 2003; Gao ZG et al., “Allosteric modulation of the adenosine family of receptors,” Mini Rev Med Chem. 5(6):545-53, 2005 Jun; Headrick J P et al., “A3 adenosine receptor-mediated protection of the ischemic heart,” Vascul Pharmacol. 42(5-6):271-9, 2005 April-May, Epub 2005 Apr 19;Hutchinson S A et al., “A(1) adenosine receptor agonists: medicinal chemistry and therapeutic potential,” Curr Pharm Des. 10(17):2021-39, 2004; Cerqueira M D, “The future of pharmacologic stress: selective A2A adenosine receptor agonists,” Am J Cardiol. 94(2A):33D-40D, 2004 Jul 22, discussion 40D-42D; Lukashev D et al., “Targeting hypoxia—A(2A) adenosine receptor-mediated mechanisms of tissue protection,” Drug Discov Today. 9(9):403-9, 2004 May 1; Yan L et al., “Adenosine receptor agonists: from basic medicinal chemistry to clinical development, “Expert Opin Emerg Drugs. 8(2):537-76, 2003 November; Sullivan G W, “Adenosine A2A receptor agonists as anti-inflammatory agents,” Curr Opin Investig Drugs. 4(11):1313-9, 2003 November; Jacobson K A et al., “Adenosine receptors as therapeutic targets, Nat Rev Drug Discov. 5(3):247-64, 2006 March; Gross, G. J. and Auchampach, J. A. “Reperfusion injury: Does it exist?” J. Mol. Cell. Cardiol. (2006), 42: 12 ; Baraldi, P. G. et al., “Ligands for A2B adenosine receptor subtype” Curr. Med. Chem. (2006) 13:3467; Yuzlenko, O.; Kiec-Kononowicz, K. “Potent adenosine A1 and A2A receptors antagonists: Recent developments.” Curr. Med. Chem. (2006), 13:3609, Cronstein, B. N. Adenosine receptors and wound healing, revised.” The Scientific World Journal. (2006) 6:984; Akkari, R. et al., “Recent progress in the development of Adenosine receptor ligands antiinflammatory drugs.” Curr. Top. Med. Chem. (2006) 6:1375; Vallon, V. et al., “Adenosine and kidney function.” Physiol. Rev. (2006) 86:901; Bours, M. J. L., et al., “Adenosine 5′-triphosphate and adenosine as endogenous signaling molecules in immunity and inflammation.” Pharmacol. Ther. (2006) 112:358.

The actions of adenosine are mediated through four receptors subtypes (A₁, A_{2A}, A_{2B} and A₃). Adenosine acts at the A₁ receptor subtype to cause decreases in heart rate, force of contraction, and responsiveness to adrenaline, and at the A_{2A} receptor subtype to cause dilation of coronary arteries to enhance blood flow to the heart. In the central nervous system (CNS), adenosine, released during episodes of epilepsy or as a consequence of hypoxia or stroke, acts at the A₁ receptor subtype to exert a neuroprotective action by decreasing electrical excitability, inhibiting the release of excitatory amino acids (EAA) and acts at the A_{2A} receptor subtype to increase cerebral blood flow. The A₃ receptor subtype is associated with cardioprotective effects.

The use of adenosine itself or non-selective agonists stimulates all receptor subtypes. This may have a desired effect, but it may also cause unwanted side effects. A variety of sub-type selective adenosine agonists have been developed, which have a desired effect but avoid potentially harmful side effects. Accordingly, in particular embodiments, adenosine agonists of the present invention selectively target any one of the receptor subtypes, or are non-selective and target two or more receptor subtypes. In particular embodiments, an adenosine agonist is selective for the A₁, A_{2A}, A_{2B}, or A₃ adenosine receptor subtype. In addition, agonists may be either full agonists or partial agonists. A partial agonist is a low-efficacy ligand that, in contrast to a full agonist, elicits only a submaximal response when occupying all (>95%) available receptors. The present invention contemplates the use of full and partial agonists, including, e.g., partial agonists selective for any subtype, including, e.g., A₁ partial agonists.

A variety of agonists contemplated by the present invention are described in Yan et al. 8-Butylamino-adenosine is a partial agonist for the adenosine A₁ receptor. Prototypical A₂ agonists include 2-[p-(2-carboxyethyl)phenethyl-amino]-5′-N-ethylcarboxamidoadenosine (CGS-2 1680) and HENECA. Another example of a NECA modified adenosine analog is 4-(3-[6-amino-9-(5-ethylcarbamoyl-3,4-dihydroxy-tetrahydro-furan-2-yl)-9H-purin-2-yl]-prop-2-ynyl)-cyclohexanecarboxylic acid methyl ester) from Adenosine Therapeutics. Agonists selective for the A₁ adenosine receptor subtype include, but are not limited to, CPA, CCPA, S(−)-ENBA, ADAC, AMP579, NNC-21-0136, GR79236, CVT-510 (Tecadenoson), SDZ WAG 994, and Selodenoson. Agonists selective for the A_{2A} adenosine receptor subtype include, but are not limited to, NECA, CGS21680, DPMA, Binodenoson, ATL-146e, and CV-3146. Agonists selective for the A_{2B} adenosine receptor subtype include, but are not limited to, LUF5835. Agonists selective for the A3 adenosine receptor subtype include, but are not limited to, IB-MECA, Cl-IB-MECA, LJ568, CP-608039, MRS3558, and MRS1898. In various embodiments, the agonist is AmP579, a mixed adenosine agonist with both A1 and A2 effects (Rhone-Poulenc Rorer, Collegeville, Pa.). The analog may also be DPMA, a selective adenosine A₂ receptor agonist, CPA, a selective adenosine A₁ receptor agonist, Benzyl-NECA, a selective A₃ receptor agonist, 2-chloroadenosine, a non-selective A₂/A₁ agonist, or NECA, a non-selective A₂/A₁ agonist.

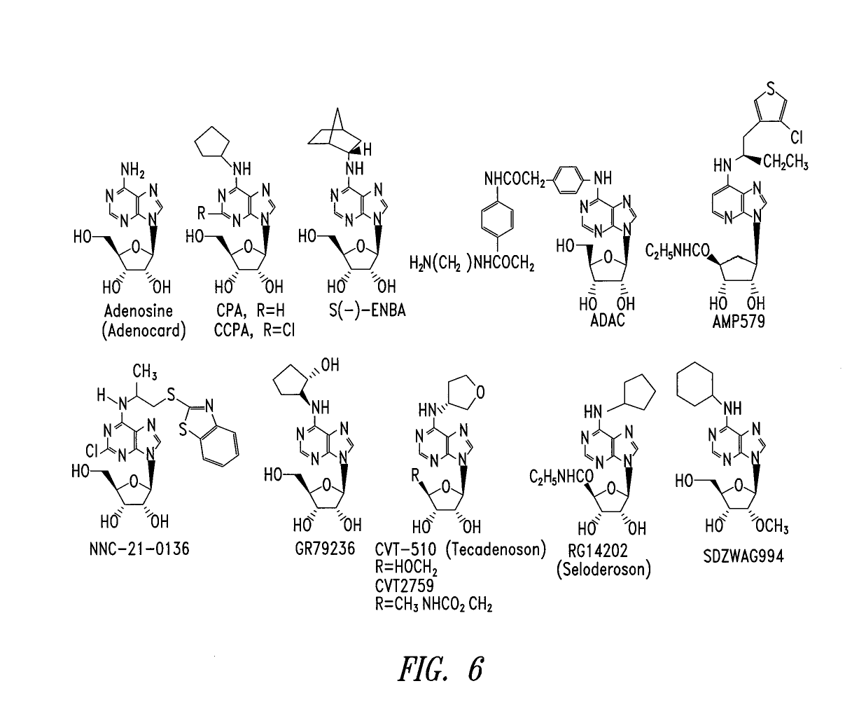

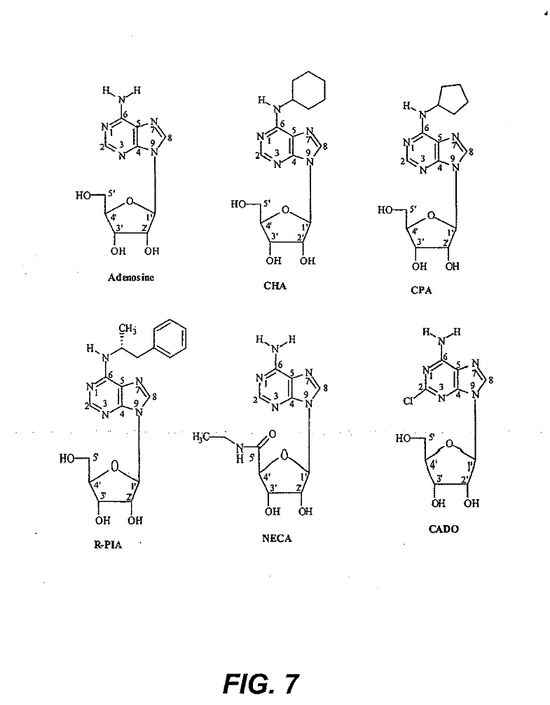

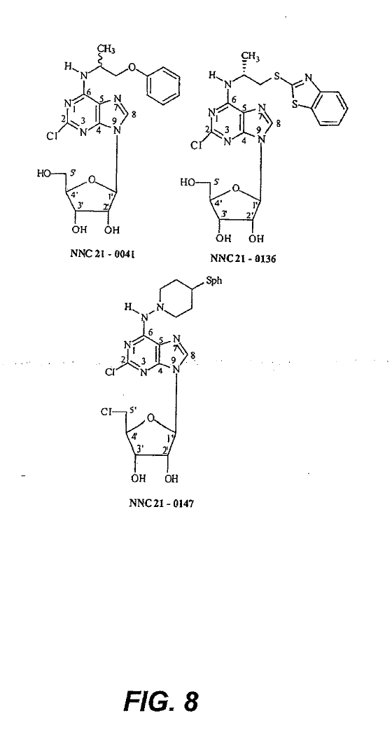

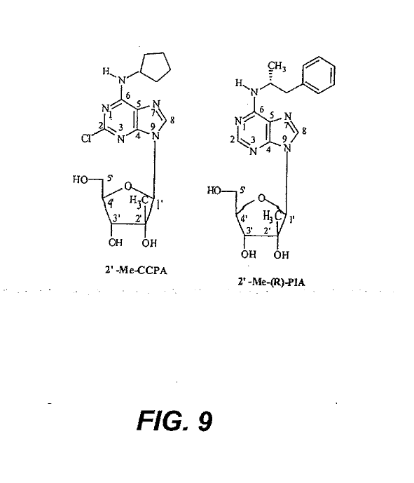

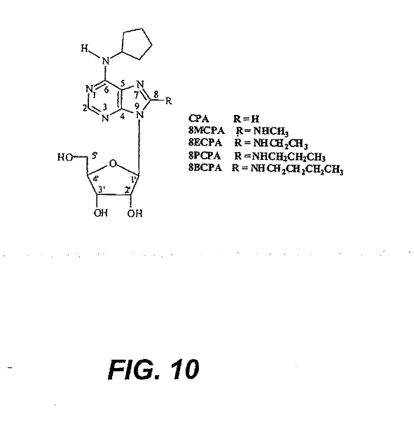

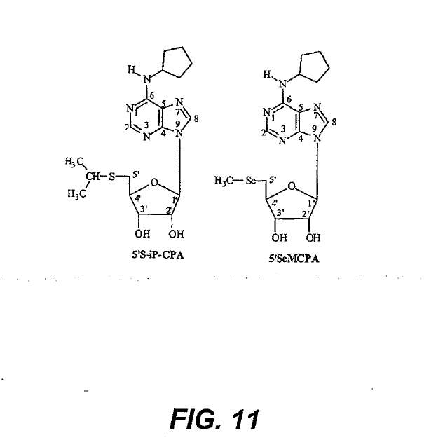

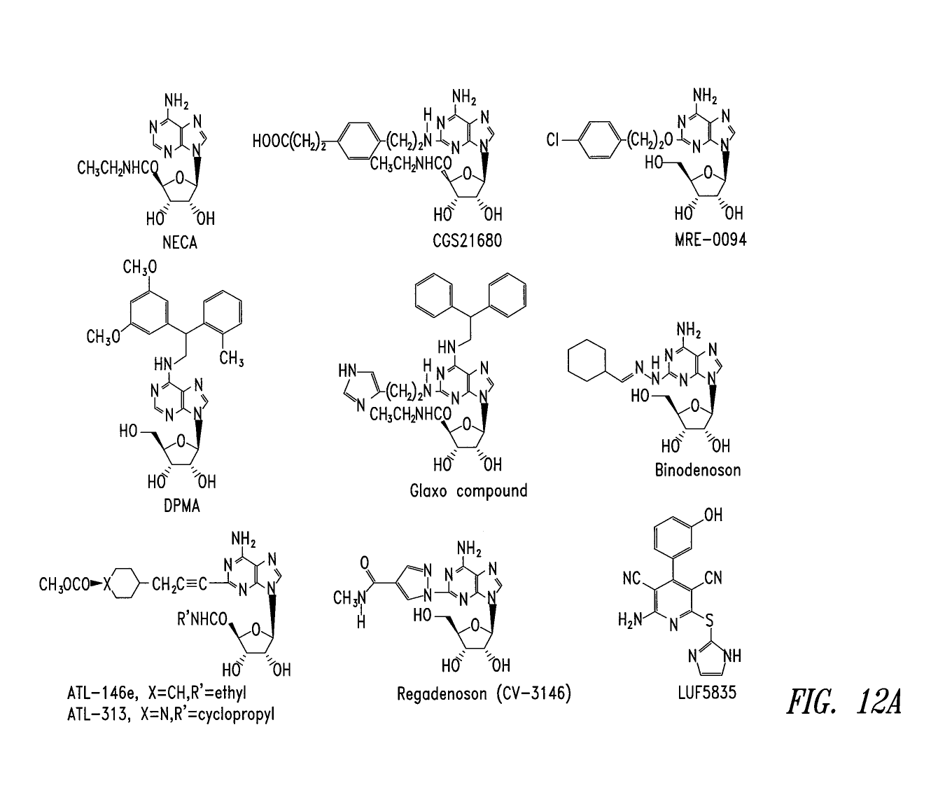

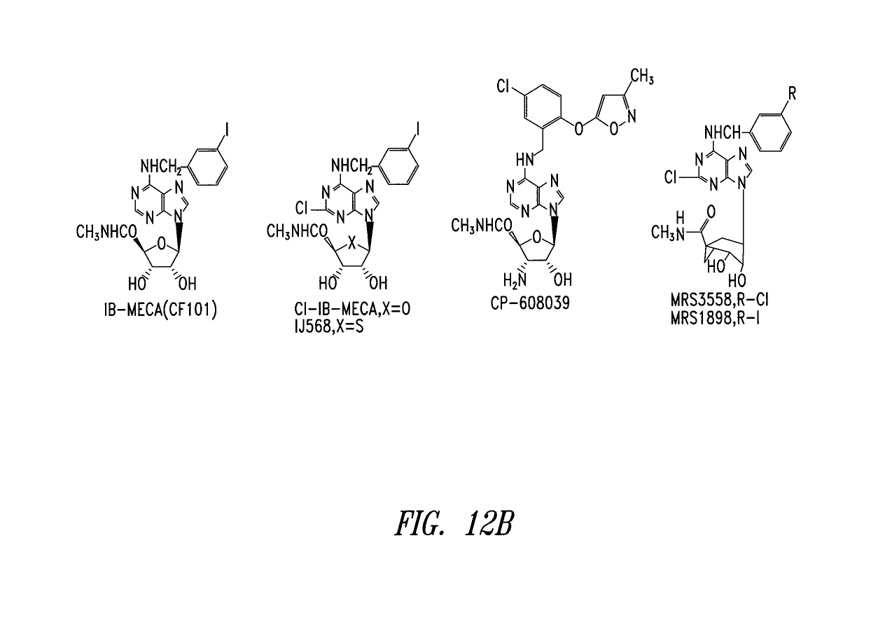

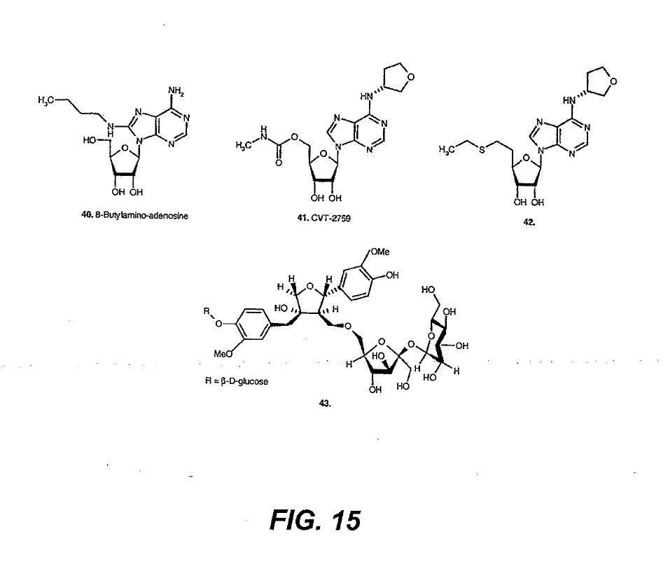

The structures of adenosine and exemplary A₁ selective agonists are shown in FIGS. 6-11 and 15. The structures of selected A_{2A} and A_{2B} selective agonists are shown in FIG. 12A, and structures of selected A₃ selective agonists are shown in FIG. 12B. These compounds are further described in Dalpiaz et al. and Jacobson et al. Interestingly, structure-activity studies have established a relationship governing agonist structure and receptor subtype selectivity, as described in Hutchinson et al. and shown in Table 1 below. The potency and selectivity of particular adenosine receptor ligands are provided in Table 1 of Muller.

TABLE 1
Adenosine Structure-Activity Relationships Governing
Receptor Subtype Selectivity

| | Adenosine Receptor Subtype | | |
|---|---|---|---|
| 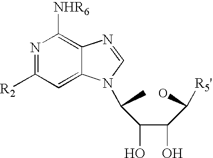 | A₁ A_{2A} A₃ | | |
| R₂ | H or X | 1-3 linker | H or Cl |
| | | with | |
| | | cycloalkyl or | |
| | | phenyl | |
| R_{5′} | CH₂OH | CH₂OH′ | C(O)NHR |
| | CH₂Cl | C(O)NHR | |
| R_{6 cycloalkyl} | H | H | |
| | substituted | substituted | substituted |
| | phenyl | phenyl | benzyl |

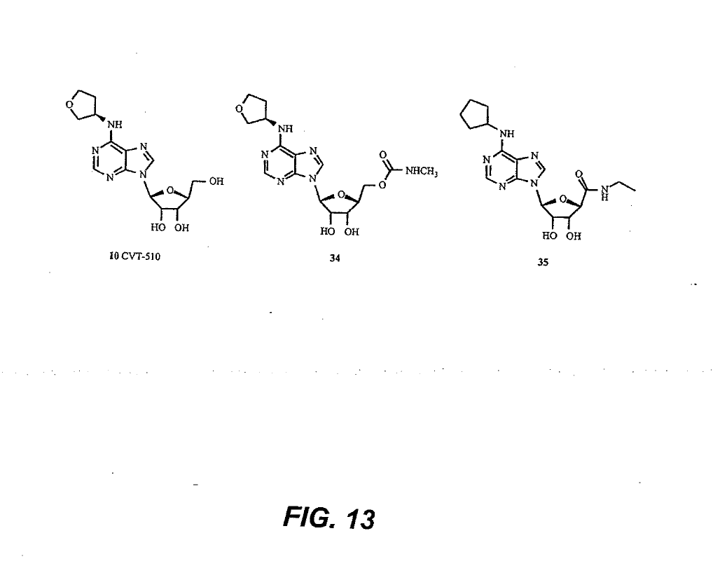

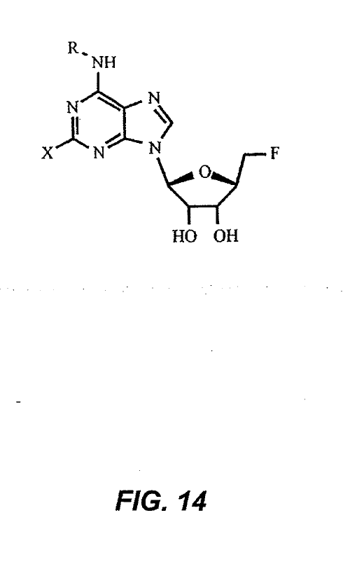

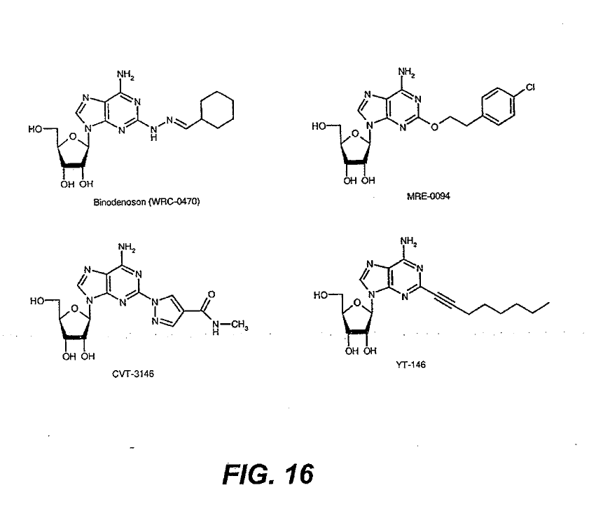

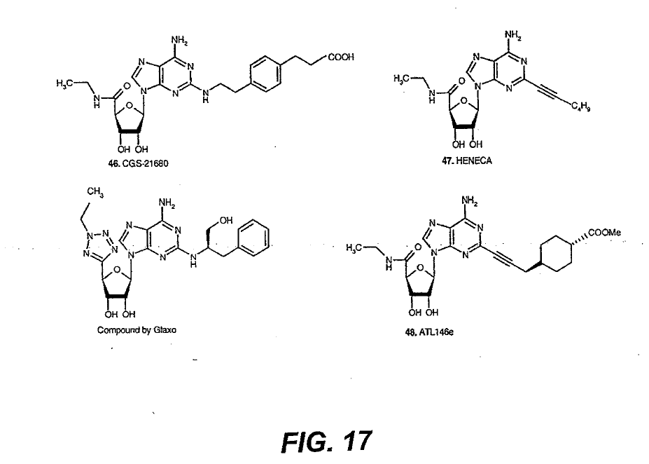

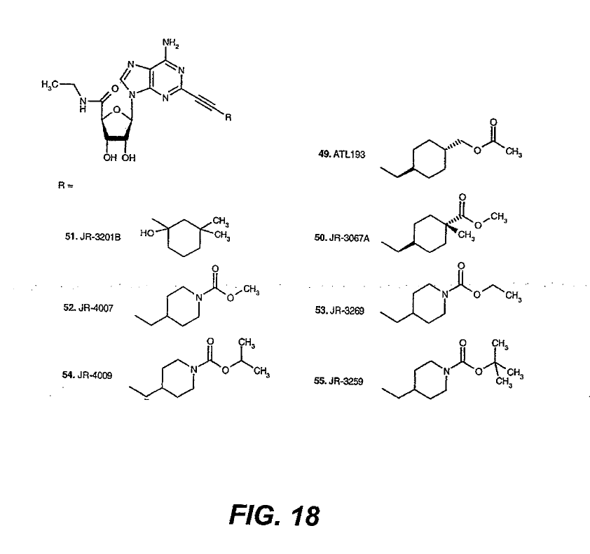

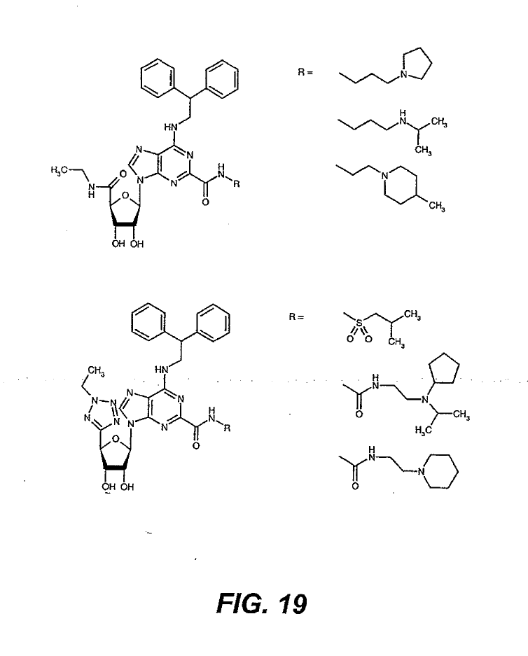

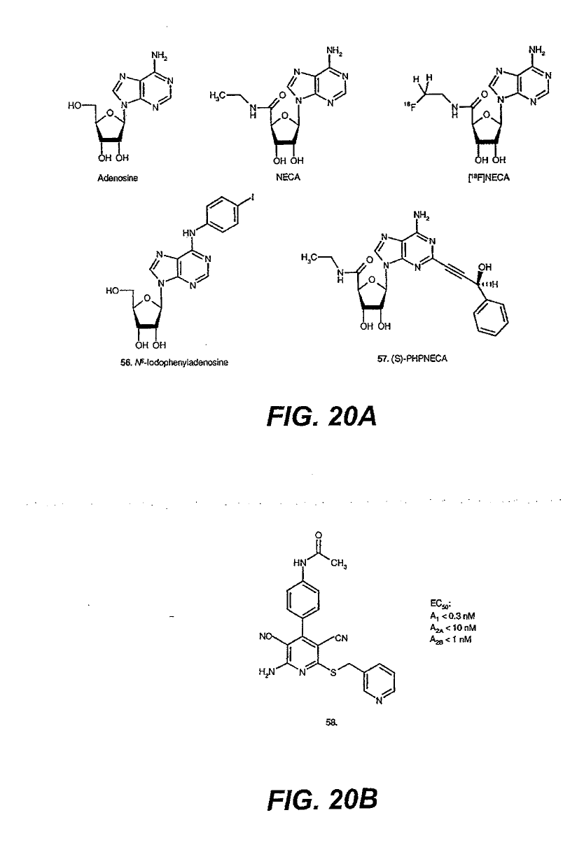

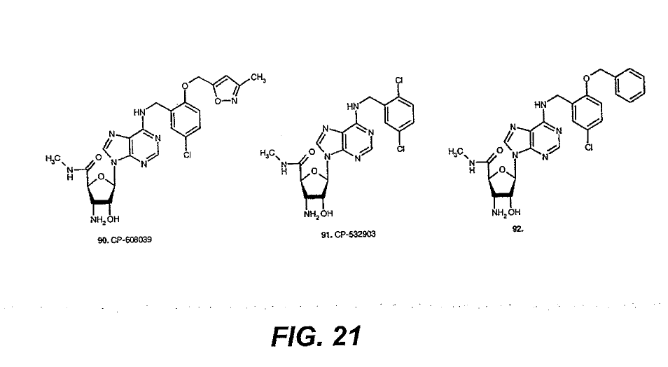

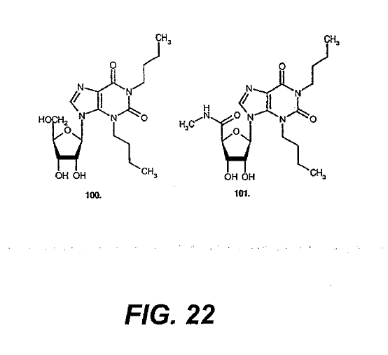

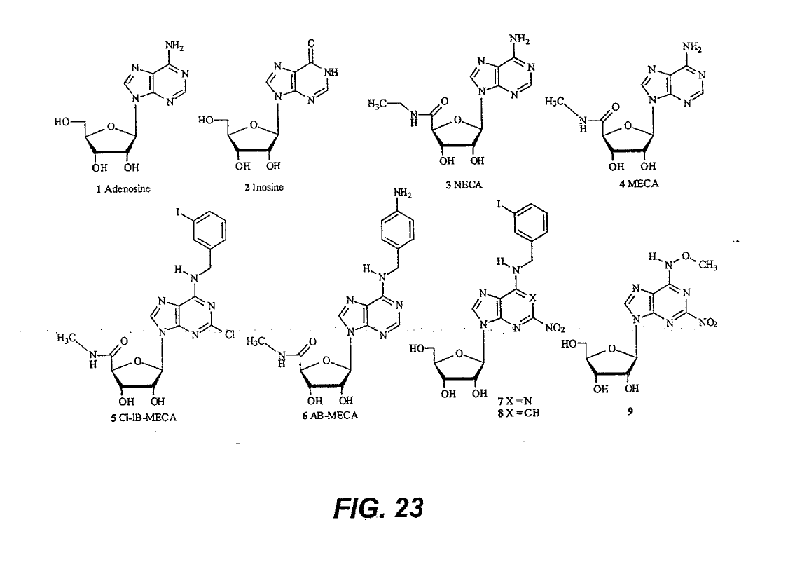

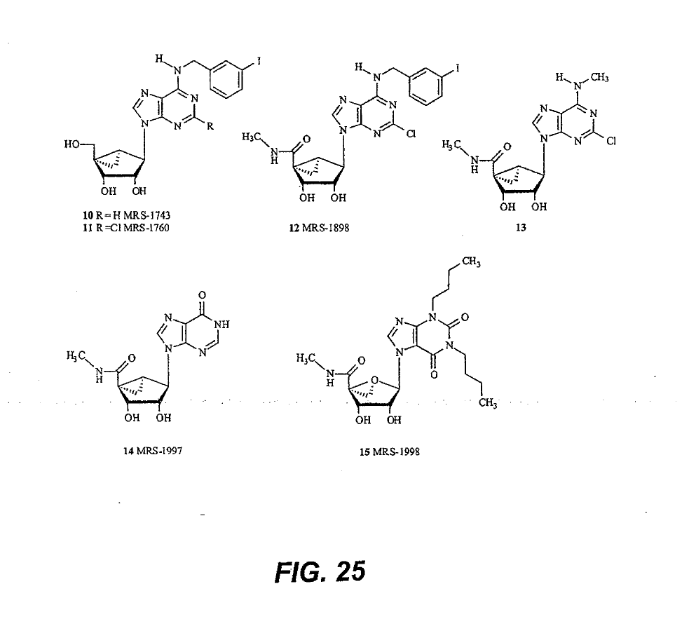

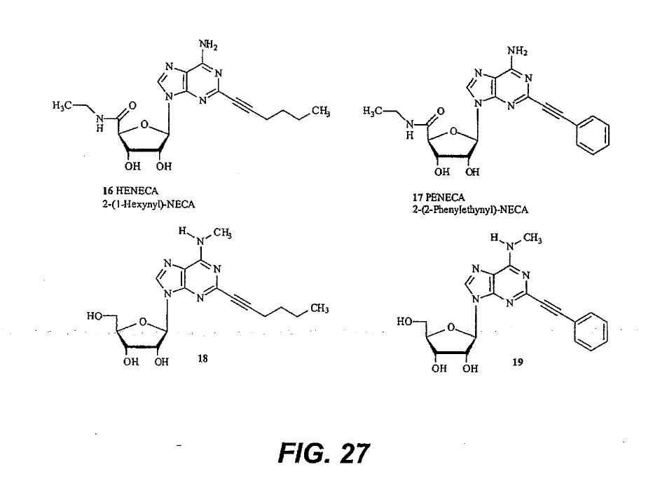

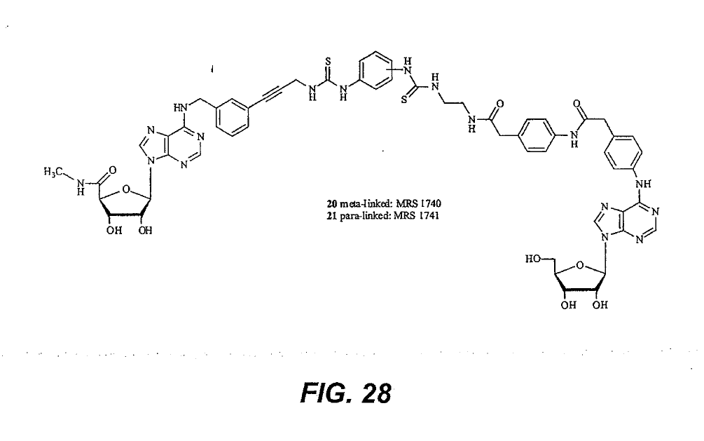

Structures of additional exemplary adenosine derivatives and adenosine receptor agonists are shown in FIGS. 13-23, 25, 27, and 28, and further described in Jacobson et al., Yan et al., and Muller.

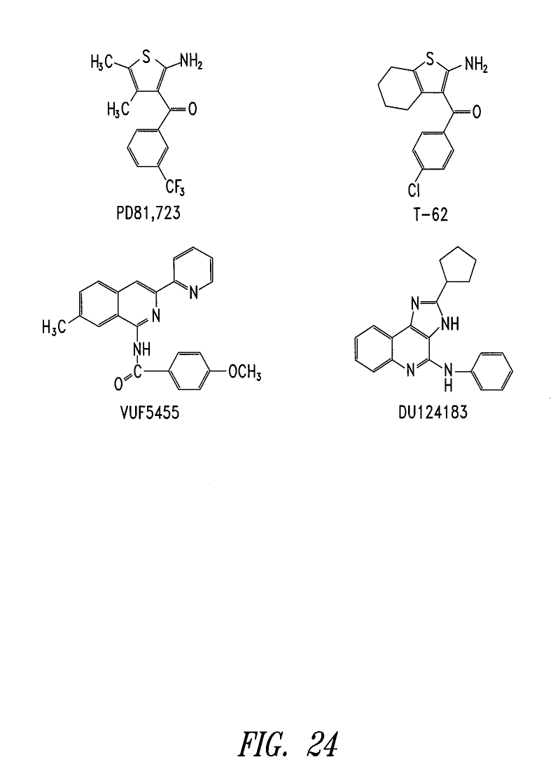

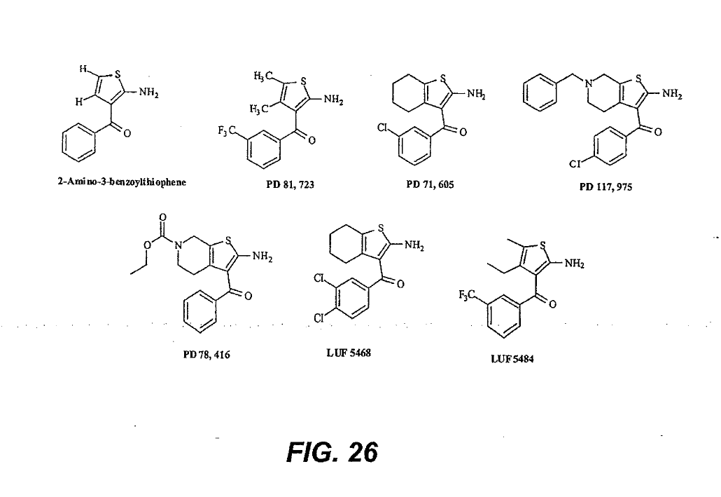

In further embodiments of the present invention, an adenosine receptor agonist is used in combination with an allosteric enhancer of agonist action. An allosteric enhancer enhances the activity of an agonist. In particular embodiments, it does so in a subtype selective manner. For example, PD81,723 and T-62 enhance the activity of agonists acting at the A₁ adenosine receptor subtype, and VUF5455 and DU124183 enhance the activity of agonists acting at the A₃ adenosine receptor subtype. The structures of representative allosteric inhibitors are shown in FIGS. 24 and 26.

In other particular embodiments, adenosine receptor agonists include those chemical entities described in any of PCT Patent Application Publication Nos. WO 2005/117910, WO 2005/107463, WO 2005/082379, WO 2005/051298, WO 2005/028489, WO 2005/018532, WO 2005/012323, WO 2005/012309, WO 2004/078184, WO 2004/069185, WO 2004/065380, WO 2004/056181, WO 2004/016635, WO 2004/011010, WO 2004/007519, WO 2003/070739, WO 2003/061670, WO 2003/048180, WO 2003/048120, WO 2003/029264, WO 2003/014137, WO 2003/006465, WO 2002/096462, WO 2002/074056, WO 2001/062979, WO 2001/040246, WO 2001/040245, WO 2001/040244, WO 2001/040243, WO 2001/039777, WO 2001/019360, WO 2000/071558, WO 2002/036610, WO 99/34804, WO 99/24451, WO 99/24450, WO 99/24449, WO 99/21617, WO 99/06053, WO 98/50047, WO 97/24363, WO 95/11904, WO 95/02604, WO 94/23723, WO 93/11138, and WO 92/20346. The present invention contemplates the use of any chemical entity described in these applications.

In certain embodiments, compositions and methods of the present invention are practiced using a chemical entity that binds to and/or is transported into cells by nucleotide transporters. Nucleoside transporters modulate many physiological processes, ranging from cardiac contractility to platelet aggregation, by regulating access of adenosine to cell surface receptors (Shryock et al; Baldwin et al.). In addition, nucleoside transporters mediate cellular uptake of physiologic nucleosides for nucleic acid synthesis in the salvage pathways in many cell types. Nucleoside transporters also represent the route of uptake for cytotoxic nucleoside analogues used for chemotherapy of cancer and viral diseases in man (Baldwin et al.; Mackey et al.).

In mammalian cells, there are two major nucleoside transporter gene families: the equilibrative nucleoside transporters (ENTs) and the concentrative nucleoside transporters (CNTs). The ENTs are facilitated carrier proteins, and the CNTs are Na(+)-dependent secondary active transporters. Mammalian nucleoside transporters are further described in Kong et al.

ENTs are ubiquitously distributed in mammalian tissues, and play important roles in a host of physiological processes. They can be divided into two classes on the basis of their sensitivity or resistance to the transport inhibitor nitrobenzylthioinosine (NBMPR). The equilibrative sensitive or es-type transporters are potently inhibited by NBMPR, with Kᵢ values in the nanomolar range. In contrast, the equilibrative insensitive or ei-type transporters are not much affected by NBMPR concentrations below 1 μM. One example of an es-type transporter is human equilibrative nucleoside transporter 1 (hENT1) (Griffiths et al.). HENT1 is a member of a novel transporter family, the ENT family, which is widely distributed in mammals, plants, yeasts, insects, nematodes and protozoans, but is apparently absent from prokaryotes (Hyde et al.).

In addition to equilibrative nucleoside transporters, active, sodium-linked uptake systems for nucleosides are found in many mammalian tissues, being especially abundant in intestine, kidney and liver. These transport activities have been divided into three classes on the basis of their substrate-specificity—the cit class are pyrimidine-selective, the cif class are purine-selective and the cib class exhibit broad substrate selectivity. All these active transporters belong to a second novel protein family designated the concentrative nucleoside transporter (CNT) family. The cib-type transporters (designated CNT3) are members of a subfamily of the CNTs, most closely related to a transporter from the most primitive living vertebrate, the hagfish. Additional representatives of the CNT family have been identified in many other eukaryotes, including nematodes and insects, as well as in many prokaryotes. Interestingly, different CNT subfamilies exhibit different stoichiometries of ion symport: the mammalian cit-and cif-type transporters (CNT1 and CNT2 respectively) transport 1 Na⁺ per nucleoside, CNT3 transports 2 Na⁺ ions, and the bacterial transporter NupC transports H⁺ ions rather than sodium. Other CNTs include N4 and N5, as described in Kong et al. The present invention contemplates the use of chemical entities, e.g., compounds, that specifically bind and/or are actively transported into a cell by any of the nucleoside transporters described herein, including, e.g., hENT1, rENT1, mENT1, hENT2, rENT2, mENT2, hENT3, mENT3, hENT4, hCNT1, rCNT1, pkCNT1, hCNT2 (hSPNT), mCNT2, rbCNT2 (rbSPNT1), hCNT3, mCNT3, and homologs, including human homologs, thereof.

Nucleoside transporters are further described in Thorn J A, et al., “Adenosine transporters,” Gen Pharmacol. 27(4):613-20, 1996 June; Baldwin S A et al., “The equilibrative nucleoside transporter family, SLC29,” Pflugers Arch. 447(5):735-43, 2004 February, Epub 2003 Jun. 28; Gray J H et al., “The concentrative nucleoside transporter family, SLC28,” Pflugers Arch. 447(5):728-34, 2004 February, Epub 2003 Jul. 11; Kong W et al., “Mammalian nucleoside transporters,” Curr Drug Metab. 5(1):63-84, 2004 Feb; Noji T et al., “Adenosine uptake inhibitors,” Eur J Pharmacol. 8;495(1):1-16, 2004 July; and Podgorska M et al., “Recent advances in studies on biochemical and structural properties of equilibrative and concentrative nucleoside transporters,” Acta Biochim Pol. 52(4):749-58, 2005, Epub 2005 Oct. 25.

The present invention contemplates the use of chemical entities, e.g., compounds that bind to and/or are transported by any nucleoside transporter, including but not limited to those described herein. In one embodiment, a chemical entity that binds to and/or is transported by a nucleoside transporter is identified according to methods described in PCT Application Publication No. WO 03/087354. In particular embodiments, chemical entities that bind to and/or are taken up by a nucleoside transporter have a biological or physiological effect upon biological material with which it is contacted. Examples of such effects include reduced body temperature and reduced metabolism. In one embodiment, the chemical entity causes biological material to enter a hypometabolic state.

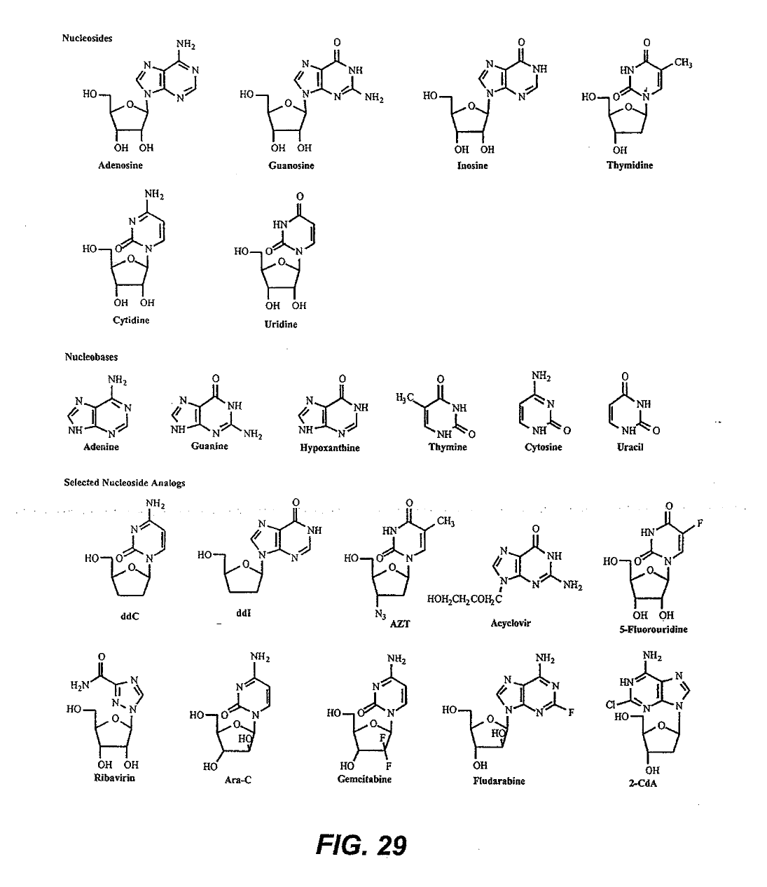

Examples of structures, i.e., nucleosides and nucleoside analogs, that bind to nucleoside transporters and may be used according to the present invention are shown in FIG. 29.

In one embodiment, the present invention contemplates the use of substituted pyrazolopyrimidines and thiazolopyrimidines, including those described in PCT Patent Application Publication No. WO 02/072585. These include compounds having the general structure shown in Formulas VIIIa, VIIIb, and IX. 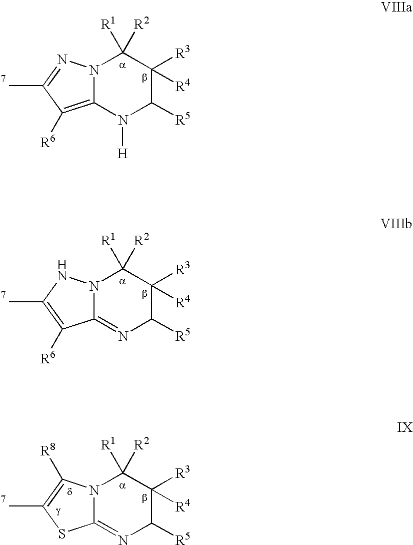
wherein
R¹ and R² independently of one another denote H, O—R⁹, S—R¹⁰, C₁₋₁₂-alkyl, C₃₋₈-cycloalkyl, —CH₂—C₃₋₈-cycloalkyl, aryl, —(C₁₋₆-alkyl)-aryl, heterocyclyl or —(C₁₋₆alkyl)-heterocyclyl, in which one of the radicals R¹ and R² is H and the other radical of R¹ and R² is not H, or in the case that one of the radicals R¹ and R² denotes aryl, the other radical of R¹ and R² denotes H or C₁₋₁₂-alkyl,
R³ and R⁴ denote H, C₁₋₁₂-alkyl, C₃₋₈-cycloalkyl, —CH₂—C₃₋₈-cycloalkyl, aryl or —(C₁₋₆-alkyl)-aryl in which at least one of the radicals R³ and R⁴ is H, or
one of the radicals R¹ and R² together with one of the radicals R³ and R⁴ forms W, where W denotes α′-(CH₂)-β′ where n=3, 4, 5 or 6, α′-CH═CH—CH₂-β′, α′-CH₂—CH═CH-β′, α′-CH═CH—CH₂—CH₂-β′, α′-CH₂—CH═CH—CH₂-β′, α′-CH₂—CH₂—CH═CH-β′, 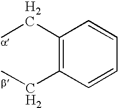
   α′-O—(CH2)ₘ-β′ where m=2, 3, 4 or 5, 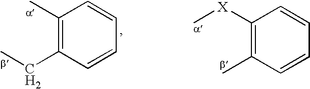
   where X═CH₂, O or S, 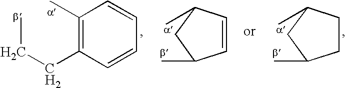
   the end of W identified by α′ is joined to the atom of the compound of the general structure (I A), (I B) or (II) identified by α, and the end of W identified by β is joined to the atom of the compound of the general structure (I A), (I B) or (II) identified by β, the other radical of R¹ and R² is H or C₁₋₁₂-alkyl, and the other radical of R³ and R⁴ is H or C₁₋₁₂-alkyl;
R⁵ denotes C₁₋₁₂-alkyl, C₃₋₈-cycloalkyl, —CH₂—C₃₋₈-cycloalkyl, aryl, —(C₁₋₆ alkyl)-aryl, heterocyclyl, —(C₁₋₆ alkyl)-heterocyclyl or C(═O)R¹¹;
R⁶ denotes H, C₁₋₈-alkyl, —CN, fluorine, chlorine, bromine, iodine, NO₂, NH₂, NHR¹², NR¹³R¹⁴, OR¹⁵, S(O)ₚR¹⁶ where p=0, 1 or 2, —C(═O)R¹⁷ or —N═N-aryl;
R⁷ denotes H, C₁₋₈-alkyl, aryl, −CN, fluorine, chlorine, bromine, iodine, NO₂, NH₂, NHR¹², NR¹³R¹⁴, OR¹⁸, S(O)_{q}R¹⁹ where q=0, 1 or 2 or denotes C(═O)R²⁰;
R⁸ denotes H, C₁₋₈-alkyl or aryl, or p0 the radicals R⁷ and R⁸ together form Y, where Y denotes γ′-CR²¹═CR²²—CR²³═CR²⁴-δ′ and the end of Y identified by γ′ is joined to the atom of the general structure (II) identified by γ, and the end of Y identified by δ′ is joined to the atom of the general structure (II) identified by δ;
R⁹ and R¹⁰ independently of one another denote H, C₁₋₈-alkyl, C₃₋₈-cycloalkyl, —CH₂—C₃₋₈-cycloalkyl, aryl or —(C₁₋₆-alkyl)-aryl;
R¹¹ denotes H, C₁₋₈-alkyl, C₃₋₈-cycloalkyl, —CH₂—C₃₋₈-cycloalkyl, aryl or OR²⁵;
R¹² denotes C₁₋₆-alkyl or —CH₂-aryl;
R¹³ and R¹⁴ are identical or different C₁₋₆-alkyl or together denote -(CH2)ₕ- where h=4 or 5;
R¹⁵ and R¹⁶ independently of one another denote H, C₁₋₈-alkyl, C₃₋₈-cycloalkyl, —CH₂—C₃₋₈-cycloalkyl, aryl or —(C₁₋₆-alkyl) -aryl;
R¹⁷ denotes H, C₁₋₈-alkyl, C₃₋₈-cycloalkyl, —CH₂—C₃₋₈-cycloalkyl, aryl, —(C₁₋₆-alkyl)-aryl, NH₂, NHR¹², NR¹³R¹⁴ or OR²⁶;
R¹⁸ and R¹⁹ independently of one another denote H, C₁₋₈-alkyl, C₃₋₈-cycloalkyl, —CH₂—C₃₋₈-cycloalkyl, aryl or —(C₁₋₆-alkyl)-aryl;
R²⁰ denotes H, C₁₋₈-alkyl, C₃₋₈-cycloalkyl, —CH₂—C₃₋₈-cycloalkyl, aryl or —(C₁₋₆-alkyl)-aryl or OR²⁷;
R²¹, R²², R²³ and R²⁴ independently of one another denote H, fluorine, chlorine, bromine, iodine or OR²⁸;
R²⁵, R²⁶, R²⁷ and R²⁸ independently of one another denote H or C₁₋₆-alkyl, where R²⁵ does not denote H if simultaneously R¹ denotes aryl and R² denotes alkyl.

The compounds according to the invention of the general structure (VIII A), (VIII B) or (IX) in the represented form or in the form of their acid(s) or their base(s) or in the form of one of their salts, in particular one of their physiologically compatible salts, or in the form of one of their solvates, in particular the hydrates; in the form of their racemate, in the form of the pure stereoisomers, in particular enantiomers or diastereomers, or in the form of mixtures of the stereoisomers, in particular of the enantiomers or diastereomers, are present in an arbitrary mixture ratio. The compounds according to the invention, in particular the pyrazolopyrimidines (VIII) according to the invention, may be present in tautomeric forms, in the case of (VIII) in the forms (VIII A) and (VIII B), wherein the optionally preferred tautomeric form may vary from compound to compound and for example depending on the aggregate state or on the chosen solvent.

“Optional” or “optionally” means that the subsequently described event of circumstances may or may not occur, and that the description includes instances where said event or circumstance occurs and instances in which it does not. For example, “optionally substituted aryl” means that the aryl radical may or may not be substituted and that the description includes both substituted aryl radicals and aryl radicals having no substitution.

2. Active Compounds

In certain embodiments, methods of the present invention comprise contacting biological material with a combination of adenosine and one or more additional active compounds. In other embodiments, particular methods of the present invention comprise contacting non-living biological material or non-biological material with an active compound.

As used herein, an “active compound” is a compound that can act on biological material to produce any of a number of effects, including, but not limited to, enhancing or increasing survivability under hypoxic or ischemic conditions, preventing cell or tissue damage due to ischemia or hypoxia, inducing a hypometabolic state, and/or achieving any of the therapeutic applications discussed herein. Accordingly, adenosine is itself an active compound. A variety of active compounds are described, e.g., in U.S. Ser. No. 60/673037, filed Apr. 20, 2005; U.S. Ser. No. 60/673295, filed Apr. 20, 2005; U.S. Ser. No. 60/713073, filed Aug. 31, 2005 and U.S. Ser. No. 60/762462, filed Jan. 26, 2006.

In some embodiments, an active compound is an oxygen antagonist, which may act directly or indirectly. Oxygen metabolism is a fundamental requirement for life in aerobic metazoans. Aerobic respiration accounts for the vast majority of energy production in most animals and also serves to maintain the redox potential necessary to carry out important cellular reactions. In hypoxia, decreased oxygen availability results in inefficient transfer of electrons to molecular oxygen in the final step of the electron transport chain. This inefficiency results in both a decrease in aerobic energy production and an increase in the production of damaging free radicals, mainly due to the premature release of electrons at complex IlIl and the formation of O₂⁻ by cytochrome oxidase (Semenza, 1999). Limited energy supplies and free radical damage can interfere with essential cellular processes such as protein synthesis and maintenance of membrane polarities (Hochachka et al., 1996) and ultimately lead to cell death.

In other embodiments, an active compound is a protective metabolic agent. Metabolism is generally understood as referring to chemical processes (in a cell or organism) that are required for life; they involve a variety of reactions to sustain energy production and synthesize (anabolism) and break down (catabolism) complex molecules.

In one embodiment, an active compound is carbon monoxide (CO). CO can be toxic to organisms whose blood carries oxygen to sustain its survival. It may be poisonous by entering the lungs through normal breathing and displacing oxygen from the bloodstream. Interruption of the normal supply of oxygen jeopardizes the functions of the heart, brain and other vital functions of the body. However, the use of carbon monoxide for medical applications is being explored (Ryter et al., 2004). At amounts of 50 parts per million (ppm), carbon monoxide presents no symptoms to humans exposed to it. However, at 200 ppm, within two-three hours the carbon monoxide can cause a slight headache; at 400 ppm, within one to two hours it can cause a frontal headache that may become widespread within three hours; and, at 800 ppm it can cause dizziness, nausea, and/or convulsions within 45 minutes, and render the subject insensible within two hours. At levels of around 1000 ppm, an organism can expire after exposure for more than around 1-2 minutes.

In other embodiments, an active compound is a chalcogenide compound. Compounds containing a chalcogen element, those in Group 6 of the periodic table, but excluding oxides, are commonly termed “chalcogenides” or “chalcogenide compounds (used interchangeably herein). These elements are sulfur (S), selenium (Se), tellurium (Te), and polonium (Po). Common chalcogenides contain one or more of S, Se and Te, in addition to other elements. Chalcogenides include elemental forms such as micronized and/or nanomilled particles of S and Se. Chalcogenides may be provided in liquid as well as gaseous forms.

In particular embodiments of the present invention, an active compound is the chalcogenide, hydrogen sulfide. Hydrogen sulfide (H₂S) is a potentially toxic gas that is often associated with petrochemical and natural gas, sewage, paper pulp, leather tanning, and food processing. One effect, at the cellular level, appears to be inhibition of cytochrome oxidase (at least in part), inhibition of other oxidative enzymes, and the reduction in activity of oxidative phosphorylation resulting in cellular hypoxia. Typical levels of hydrogen sulfide contemplated for use in accordance with the present invention include values of about 1 to about 500 ppm, about 10 to about 400 ppm, about 50 to about 300 ppm, and about 80 to about 300 ppm, or the equivalent oral, intravenous or transdermal dosage thereof. Other relevant ranges include about 10 to about 80 ppm, about 20 to about 80 ppm, about 10 to about 70 ppm, about 20 to about 70 ppm, about 20 to about 60 ppm, and about 30 to about 60 ppm, or the equivalent oral, intravenous or transdermal thereof. It also is contemplated that, for a given animal in a given time period, the chalcogenide atmosphere should be reduced to avoid a potentially lethal build up of chalcogenide in the subject. For example, an initial environmental concentration of 80 ppm may be reduced after 30 min to 60 ppm, followed by further reductions at 1 hr (40 ppm) and 2 hrs (20 ppm). In particular embodiments, effective concentrations of hydrogen sulfide in a human are in the range of 50 ppm to 500 ppm, delivered continuously. For certain embodiments of intravenous administration, effective concentrations are in the range of 0.5 to 50 milligrams per kilogram of body weight delivered continuously.

The present invention also concerns the use of compounds and agents that produce H₂S under certain conditions, such as upon exposure, or soon thereafter, to biological matter. It is contemplated that such precursors yield H₂S upon one or more enzymatic or chemical reactions. In one embodiment, precursors of H2S include NaHS.

“Sulfide” refers to sulfur in its −2 valence state, either as H2S or as a salt thereof (e.g., NaHS, Na2S, etc.). “H2S” is generated by the spontaneous dissociation of the chalcogenide salt and H2S donor, sodium hydrosulfide (NaHS), in aqueous solution according to the equations:
NaHS→Na++HS⁻
2HS⁻←→H₂S+S₂⁻
HS⁻+H+←→H₂S.

In certain embodiments, the chalcogenide is a salt, preferably salts wherein the chalcogen is in a −2 oxidation state. Sulfide salts encompassed by embodiments of the invention include, but are not limited to, sodium sulfide (Na₂S), sodium hydrogen sulfide (NaHS), potassium sulfide (K₂S), potassium hydrogen sulfide (KHS), lithium sulfide (Li₂S), rubidium sulfide (Rb₂S), cesium sulfide (Cs₂S), ammonium sulfide ((NH₄)₂S), ammonium hydrogen sulfide (NH₄)HS, beryllium sulfide (BeS), magnesium sulfide (MgS), calcium sulfide (CaS), strontium sulfide (SrS), barium sulfide (BaS), and the like.

It is understood that in other embodiments, the present invention may be practiced using chalcogenides other than sulfur. In certain embodiments, the chalcogenide compound comprises sulfur, while in others it comprises selenium, tellurium, or polonium. In certain embodiments, a chalcogenide compound contains one or more exposed sulfide groups. In particular embodiments, it is contemplated that this chalcogenide compound contains 1, 2, 3, 4, 5, 6 or more exposed sulfide groups, or any range derivable therein. In particular embodiments, such a sulfide-containing compound is CS₂ (carbon disulfide).

In other embodiments, as active compound is a chalcogenide precursor. “Chalcogenide precursor” refers to compounds and agents that can yield a chalcogenide, e.g., hydrogen sulfide (H₂S), under certain conditions, such as upon exposure, or soon thereafter, to biological matter. Such precursors yield H₂S or another chalcogenide upon one or more enzymatic or chemical reactions. In certain embodiments, the chalcogenide precursor is dimethylsulfoxide (DMSO), dimethylsulfide (DMS), methylmercaptan (CH₃SH), mercaptoethanol, thiocyanate, hydrogen cyanide, methanethiol (MeSH), or carbon disulfide (CS₂). In certain embodiments, the chalcogenide precursor is CS₂, MeSH, or DMS.

Compounds on the order of the size of these molecules are particularly contemplated (that is, within about 50% of their molecular weights).

In certain embodiments of the invention, an active compound has a chemical structure as set forth as Formula I or IV described herein, or is a precursor of Formula I or IV.

A variety of chemical structures and compounds are described herein. The following definitions apply to terms used to describe these structures and compounds discussed herein:

“Alkyl,” where used, either alone or within other terms such as “arylalkyl”, “aminoalkyl”, “thioalkyl” “cyanoalkyl” and “hydroxyalkyl”, refers to linear or branched radicals having one to about twenty carbon atoms. The term “lower alkyl” refers to C₁-C₆ alkyl radicals. As used herein the term alkyl includes those radicals that are substituted with groups such as hydroxy, halo (such as F, Cl, Br, I), haloalkyl, alkoxy, haloalkoxy, alkylthio, cyano, isocyano, carboxy (—COOH), alkoxycarbonyl, (—COOR), acyl, acyloxy, amino, alykamino, urea (—NHCONHR), thiol, alkylthio, sulfoxy, sulfonyl, arylsulfonyl, alkylsulfonyl, sulfonamido, arylsulfonamido, heteroaryl, heterocyclyl, heterocycloalkyl, amidyl, alkylimino carbonyl, amidino, guanidono, hydrazino, hydrazide, sodium sulfonyl (—SO₃Na), sodium sulfonylalkyl (—R SO₃Na). Examples of such radicals include, but are not limited to, methyl, ethyl, n-propyl, isopropyl, n-butyl, isobutyl, sec-butyl, tert-butyl, pentyl, iso-amyl, hexyl and the like.

“Hydroxyalkyl” refers to an alkyl radical, as defined herein, substituted with one or more hydroxyl radicals. Examples of hydroxyalkyl radicals include, but are not limited to, hydroxymethyl, 2-hydroxyethyl, 2-hydroxypropyl, 3-hydroxypropyl, 2-hydroxybutyl, 3-hydroxybutyl, 4-hydroxybutyl, 2,3-dihydroxypropyl, 1-(hydroxymethyl)-2-hydroxyethyl, 2,3-dihydroxybutyl, 3,4-dihydroxybutyl, and 2-(hydroxymethyl)-3-hydroxypropyl, and the like.

“Arylalkyl” refers to the radical R′R-wherein an alkyl radical, “R” is substituted with an aryl radical “R′.” Examples of arylalkyl radicals include, but are not limited to, benzyl, phenylethyl, 3-phenylpropyl, and the like.

“Aminoalkyl” refers to the radical H₂NR′—, wherein an alkyl radical is substituted with am amino radical. Examples of such radicals include aminomethyl, amino ethyl, and the like. “Alkylaminoalkyl” refers to an alkyl radical substituted with an alkylamino radical.

“Alkylsulfonamido” refers to a sulfonamido group (—S(O)₂—NRR′) appended to an alkyl group, as defined herein.

“Thioalkyl” refers to wherein an alkyl radical is substituted with one or more thiol radicals. “Alkylthioalkyl” refers to wherein an alkyl radical is substituted with one or more alkylthio radicals. Examples include, but are not limited to, methylthiomethyl, ethylthioisopropyl, and the like. Arylthioalkyl” refers to wherein an alkyl radical, as herein defined, is substituted with one or more arylthio radicals.

“Carboxyalkyl” refers to the radicals —RCO₂H, wherein an alkyl radical is substituted with a carboxyl radical. Example include, but are not limited to, carboxymethyl, carboxyehtyl, carboxypropyl, and the like.

“Alkylene” refers to bridging alkyl radicals.

The term “alkenyl” refers to an unsaturated, acyclic hydrocarbon radical in so much as it contains at least one double bond. Such alkenyl radicals contain from about 2 to about 20 carbon atoms. The term “lower alkenyl” refers to C₁-C₆ alkenyl radicals. As used herein, the term alkenyl radicals includes those radicals substituted as for alkyl radicals. Examples of suitable alkenyl radicals include propenyl, 2-chloropropenyl, buten-1-yl, isobutenyl, pent-1-en-1-yl, 2-2-methy-1-buten-1-yl, 3-methyl-1-buten-1-yl, hex-2-en-1-yl, 3-hydroxyhex-1-en-1-yl, hept-1-en-1-yl, and oct-1-en-1-yl, and the like.

The term “alkynyl” refers to an unsaturated, acyclic hydrocarbon radical in so much as it contains one or more triple bonds, such radicals containing about 2 to about 20 carbon atoms. The term “lower alkynyl” refers to C₁-C₆ alkynyl radicals. As used herein, the term alkynyl radicals includes those radicals substituted as for alkyl radicals. Examples of suitable alkynyl radicals include ethynyl, propynyl, hydroxypropynyl, but-1-yn-1-yl, but-1-yn-2-yl, pent-1-yn-1-yl, pent-1-yn-2-yl, 4-methoxypent-1-yn-2-yl, 3-methylbut-1-yn-1-yl, hex-1-yn-1-yl 1-yn-2-yl, hex-1-yn-3-yl, 3,3-dimethyl-1-butyn-1-yl radicals and the like

“Alkoxy,” refers to the radical R′O—, wherein R′ is an alkyl radical as defined herein. Examples include, but are not limited to, methoxy, ethoxy, propoxy, butoxy, isopropoxy, tert-butoxy alkyls, and the like. Alkoxyalkyl” refers to alkyl radicals substituted by one or more alkoxy radicals. Examples include, but are not limited to, methoxymethyl, ethoxyethyl, methoxyethyl, isopropoxyethyl, and the like.

“Alkoxycarbonyl” refers to the radical R—O—C(O)—, wherein R is an alkyl radical as defined herein. Examples of alkoxycarbonyl radicals include, but are not limted to, methoxycarbonyl, ethoxycarbonyl, sec-butoxycarbonyl, isoprpoxycarbonyl, and the like. Alkoxythiocarbonyl refers to R—O—C(S)—.

“Aryl” refers to the monovalent aromatic carbocyclic radical consisting of one individual ring, or one or more fused rings in which at least one ring is aromatic in nature, which can optionally be substituted with one or more, preferably one or two, substituents such as hydroxy, halo (such as F, Cl, Br, I), haloalkyl, alkoxy, haloalkoxy, alkylthio, cyano, carboxy (—COOH), alkoxycarbonyl, (—COOR), acyl, acyloxy, amino, alykamino, urea (—NHCONHR), thiol, alkylthio, sulfoxy, sulfonyl, arylsulfonyl, alkylsulfonyl, sulfonamido, arylsulfonamido, heteroaryl, heterocyclyl, heterocycloalkyl, amidyl, alkylimino carbonyl, amidino, guanidono, hydrazino, hydrazide, sodium sulfonyl (—SO₃Na), sodium sulfonylalkyl (—R SO₃Na), unless otherwise indicated. Alternatively two adjacent atoms of the aryl ring may be substituted with a methylenedioxy or ethylenedioxy group. Examples of aryl radicals include, but are not limited to, phenyl, naphthyl, biphenyl, indanyl, anthraquinolyl, tert-butyl-phenyl, 1,3-benzodioxolyl, and the like.

“Arylsulfonamido” refers to a sulfonamido group, as defined herein, appended to an aryl group, as defined herein.

“Thioaryl” refers to an aryl group substituted with one or more thiol radicals.

“Alkylamino” refers to amino groups that are substituted with one or two alkyl radicals. Examples include monosubstituted N-alkylamino radicals and N,N-dialkylamino radicals. Examples include N-methylamino, N-ethylamino, N,N-dimeythylamino N,N-diethylamino, N-methyl, N-ethyl-amino, and the like.

“Aminocarbonyl” refers to the radical H₂NCO—. “Aminocarbonyalkyl” refers to the substitution of an alkyl radical, as herein defined, by one or more aminocarbonyl radicals.

“Amidyl” refers to RCO—NH—, wherein R is a H or aklyl, aryl, or heteroaryl, as defined herein.

“Imino carbonyl” refers to a carbon radical having two of the four covalent bond sites shared with an imino group. Examples of such imino carbonyl radicals include, for example, C═NH, C═NCH₃, C═NOH, and C═NOCH₃. The term “alkylimino carbonyl” refers to an imino radical substituted with an alkyl group, The term “amidino” refers to a substituted or unsubstituted amino group bonded to one of two available bonds of an iminocarbonyl radical. Examples of such amidino radicals include, for example, NH₂—C═NH, NH₂—C═NCH₃, NH—C═NOCH₃ and NH(CH₃)—C═NOH. The term “guanidino” refers to an amidino group bonded to an amino group as defined above where said amino group can be bonded to a third group. Examples of such guanidino radicals include, for example, NH₂—C(NH) —NH—, NH₂—C(NCH₃)—NH—, NH₂—C(NOCH₃)—NH—, and CH₃NH—C(NOH)—NH—. The term “hydrazino” refers to —NH—NRR′, where R and R′ are independently hydrogen, alkyl and the like. “Hydrazide” refers to —C(═O) —NH—NRR′.

The term “heterocyclyl” refers to saturated and partially saturated heteroatom-containing ring-shaped radicals having from 4 through 15 ring members, herein referred to as “C₄-C₁₅ heterocyclyl” selected from carbon, nitrogen, sulfur and oxygen, wherein at least one ring atom is a heteroatom. Heterocyclyl radicals may contain one, two or three rings wherein such rings may be attached in a pendant manner or may be fused. Examples of saturated heterocyclic radicals include saturated 3 to 6-membered heteromonocylic group containing 1 to 4 nitrogen atoms[e.g. pyrrolidinyl, imidazolidinyl, piperidino, piperazinyl, etc]; saturated 3 to 6-membered heteromonocyclic group containing 1 to 2 oxygen atoms and 1 to 3 nitrogen atoms [e.g. morpholinyl, etc.]; saturated 3 to 6-membered heteromonocyclic group containing 1 to 2 sulfur atoms and 1 to 3 nitrogen atoms [e.g., thiazolidinyl, etc.]. Examples of partially saturated heterocyclyl radicals include dihydrothiophene, dihydropyran, dihydrofuran and dihydrothiazole. Non-limiting examples of heterocyclic radicals include 2-pyrrolinyl, 3-pyrrolinyl, pyrrolindinyl, 1,3-dioxolanyl, 2H-pyranyl, 4H-pyranyl, piperidinyl, 1,4-dioxanyl, morpholinyl, 1,4-dithianyl, thiomorpholinyl, and the like. Such heterocyclyl groups may be optionally substituted with groups such as substituents such as hydroxy, halo (such as F, Cl, Br, I), haloalkyl, alkoxy, haloalkoxy, alkylthio, cyano, carboxy (—COOH), alkoxycarbonyl, (—COOR), acyl, acyloxy, amino, alykamino, urea (—NHCONHR), thiol, alkylthio, sulfoxy, sulfonyl, arylsulfonyl, alkylsulfonyl, sulfonamido, arylsulfonamido, heteroaryl, heterocyclyl, heterocycloalkyl, amidyl, alkylimino carbonyl, amidino, guanidono, hydrazino, hydrazide, sodium sulfonyl (—SO₃Na), sodium sulfonylalkyl (—RSO₃Na).

“Hetroaryl” refers to monovalent aromatic cyclic radicals having one or more rings, preferably one to three rings, of four to eight atoms per ring, incorporating one or more heteroatoms, preferably one or two, within the ring (chosen from nitrogen, oxygen, or sulfur), which can optionally be substituted with one or more, preferably one or two substituents selected from substituents such as hydroxy, halo (such as F, Cl, Br, I), haloalkyl, alkoxy, haloalkoxy, alkylthio, cyano, carboxy (—COOH), alkoxycarbonyl, (—COOR), acyl, acyloxy, amino, alykamino, urea (—NHCONHR), thiol, alkylthio, sulfoxy, sulfonyl, arylsulfonyl, alkylsulfonyl, sulfonamido, arylsulfonamido, heteroaryl, heterocyclyl, heterocycloalkyl, amidyl, alkylimino carbonyl, amidino, guanidono, hydrazino, hydrazide, sodium sulfonyl (—SO₃Na), sodium sulfonylalkyl (—RSO₃Na), unless otherwise indicated. Examples of heteroaryl radicals include, but are not limited to, imidazolyl, oxazolyl, thiazolyl, pyrazinyl, thienyl, furanyl, pyridinyl, quinolinyl, isoquinolinyl, benzofuryl, benzothiophenyl, benzothiopyranyl, benzimidazolyl, benzoxazolyl, benzothiazolyl, benzopyranyl, indazolyl, indolyl, isoindolyl, quinolinyl, isoquinolinyl, naphthyridinyl, benezenesulfonyl-thiophenyl, and the like.

“Heteroaryloxy” refers to heteroaryl radicals attached to an oxy radical. Examples of such radicals include, but are not limited to, 2-thiophenyloxy, 2-pyrimidyloxy, 2-pyridyloxy, 3-pyridyloxy, 4-pyridyloxy, and the like

“Heteroaryloxyalkyl” refers to alkyl radicals substituted with one or more heteroaryloxy radicals. Examples of such radicals include 2-pyridyloxymethyl, 3-pyridyloxyethyl, 4-pyridyloxymethyl, and the like.

“Cycloalkyl” refers to monovalent saturated carbocyclic radicals consisting of one or more rings, typically one or two rings, of three to eight carbons per ring, which can typically be substituted with one or more, substitutents hydroxy, halo (such as F, Cl, Br, I), haloalkyl, alkoxy, haloalkoxy, alkylthio, cyano, carboxy (—COOH), alkoxycarbonyl, (—COOR), acyl, acyloxy, amino, alykamino, urea (—NHCONHR), thiol, alkylthio, sulfoxy, sulfonyl, arylsulfonyl, alkylsulfonyl, sulfonamido, arylsulfonamido, heteroaryl, heterocyclyl, heterocycloalkyl, amidyl, alkylimino carbonyl, amidino, guanidono, hydrazino, hydrazide, sodium sulfonyl (—SO₃Na), sodium sulfonylalkyl (—R SO₃Na), unless otherwise indicated. Examples of cycloalkyl radicals include, but are not limited to, cyclopropyl, cyclobutyl, 3-ethylcyclobutyl, cyclopentyl, cycloheptyl, and the like. “Cycloalkenyl” refers to radicals having three to ten carbon atoms and one or more carbon-carbon double bonds. Typical cycloalkenyl radicals have three to seven carbon atoms. Examples include cyclobutenyl, cyclopentenyl, cyclohexenyl, cycloheptenyl, and the like “Cycloalkenylalkyl” refers to radicals wherein an alkyl radical, as defined herein, is substituted by one or more cycloalkenyl radicals.

“Cylcoalkoxy” refers to cycloalkyl radicals attached to an oxy radical. Examples include, but are not limited to, cyclohexoxy, cyclopentoxy and the like.

“Cylcoalkoxyalkyl” refers to alkyl radicals substituted one or more cycloalkoxy radicals. Examples include cyclohexoxyethyl, cyclopentoxymethyl, and the like.

“Sulfinyl” refers to —S(O)—.

“Sulfonyl” refers to —S(O)₂—, wherein “alkylsulfonyl” refers to a sulfonyl radical substituted with an alkyl radical, RSO₂—, arylsulfonyl refers to aryl radicals attached to a sulfonyl radical. “Sulfonamido” refers to —S(O)₂—NRR′.

“Sulfonic acid” refers to —S(O)₂OH. “Sulfonic ester” refers to —S(O)₂OR, wherein R is a group such as an alkyl as in sulfonic alkyl ester.

“Thio” refers to —S—. “Alkylthio” refers to RS-wherein a thiol radical is substituted with an alkyl radical R. Examples include methylthio, ethylthio, butlythio, and the like. “Arylthio” refers to R′S—, wherein a thio radical is substituted with an aryl radical, as herein defined. “Examples include, but are not limited to, phenylthio, and the like. Examples include, but are not limited to, phenylthiomethyl and the like. “Alkylthiosulfonic acid” refers to the radical HO₃SR′S—, wherein an alkylthioradical is substituted with a sulfonic acid radical.

“Thiosulfenyl” refers to —S—SH.

“Acyl”, alone or in combination, refers to a carbonyl or thionocarbonyl group bonded to a radical selected from, for example, hydrido, alkyl, alkenyl, alkynyl, haloalkyl, alkoxy, alkoxyalkyl, haloalkoxy, aryl, heterocyclyl, heteroaryl, alkylsulfinylalkyl, alkylsulfonylalkyl, aralkyl, cycloalkyl, cycloalkylalkyl, cycloalkenyl, alkylthio, arylthio, amino, alkylamino, dialkylamino, aralkoxy, arylthio, and alkylthioalkyl. Examples of “acyl” are formyl, acetyl, benzoyl, trifluoroacetyl, phthaloyl, malonyl, nicotinyl, and the like.

The term “acylthiol” and “acyldisulfide” refers to the radicals RCOS— and RCOSS— respectively.

The term “thiocarbonyl” refers to the compounds and moieties which contain a carbon connected with a double bond to a sulfur atom —C(═S)—. “Alkylthiocarbonyl” refers to wherein a thiocarbonyl group is substituted with an alkyl radical, R. as defined herein, to form the monovalent radical RC(═S)—. “Aminothiocarbonyl” refers to a thiocarbonyl group substituted with an amino group, NH₂C(═S)—.

“Carbonyloxy” refers to —OCOR.

“Alkoxycarbonyl” refers to —COOR.

“Carboxyl” refers to —COOH.

For those compounds with stereoisomers, all stereoisomers thereof, including cis/trans geometric isomers, diastereomers and the individual enantiomers are contemplated.

Moreover, in certain methods of the present invention, an active compound has a chemical structure of (referred to as Formula I):

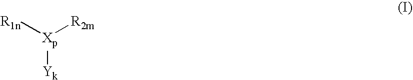

wherein X is N, O, Po, S, Se, or Te;

wherein Y is N or O;

wherein R₁ is H, C, lower alkyl, a lower alcohol, or CN;

wherein R₂ is H, C, lower alkyl, or a lower alcohol, or CN;

wherein n is 0 or 1;

wherein m is 0 or 1;

wherein k is 0, 1, 2, 3, or 4; and,

wherein p is 1 or 2.

The terms “lower alkyl” and “lower alcohol” are used according to their ordinary meanings and the symbols are the ones used to refer to chemical elements. This chemical structure will be referred to as the “reducing agent structure” and any compound having this structure will be referred to as a reducing agent structure compound. In additional embodiments, k is 0 in the reducing agent structure. Moreover, in other embodiments, the R₁ and/or R₂ groups can be an amine or lower alkyl amine. In others, R₁ and/or R₂ could be a short chain alcohol or a short chain ketone. Additionally, R₁ and R₂ may be a linear of branched chain bridg and/or the compound may be a cyclic compound. In still further embodiments, X may also be a halogen. The term “lower” is meant to refer to 1, 2, 3, 4, 5, or 6 carbon atoms, or any range derivable therein. Moreover, R₁ and/or R₂ may be other small organic groups, including, C₂-C₅ esters, amides, aldehydes, ketones, carboxylic acids, ethers, nitriles, anhydrides, halides, acyl halides, sulfides, sulfones, sulfonic acids, sulfoxides, and/or thiols. Such substitutions are clearly contemplated with respect to R₁ and/or R₂. In certain other embodiments, R₁ and/or R₂ may be short chain versions of the small organic groups discussed above. “Short chain” means 1, 2,3, 4, 5, 6, 7, 8, 9, 10, 11, or 12 carbon molecules, or any range derivable therein.

A further aspect of the invention encompasses active compounds represented by Formula II:

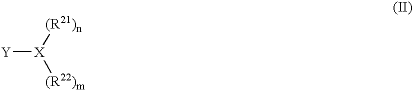

wherein:

X is N, O, P, Po, S, Se, Te, O—O, Po—Po, S—S, Se—Se, or Te—Te;

n and m are independently 0 or 1; and

wherein R²¹ and R²² are independently hydrogen, halo, cyano, phosphate, thio, alkyl, alkenyl, alkynyl, alkoxy, aminoalkyl, cyanoalkyl, hydroxyalkyl, haloalkyl, hydroxyhaloalkyl, alkylsulfonic acid, thiosulfonic acid, alkylthiosulfonic acid, thioalkyl, alkylthio, alkylthioalkyl, alkylaryl, carbonyl, alkylcarbonyl, haloalkylcarbonyl, alkylthiocarbonyl, aminocarbonyl, aminothiocarbonyl, alkylaminothiocarbonyl, haloalkylcarbonyl, alkoxycarbonyl, aminoalkylthio, hydroxyalkylthio, cycloalkyl, cycloalkenyl, aryl, aryloxy, heteroaryloxy, heterocyclyl, heterocyclyloxy, sulfonic acid, sulfonic alkyl ester, thiosulfate, or sulfonamido; and

Y is cyano, isocyano, amino, alkyl amino, aminocarbonyl, aminocarbonyl alkyl, alkylcarbonylamino, amidino, guanidine, hydrazino, hydrazide, hydroxyl, alkoxy, aryloxy, hetroaryloxy, cyloalkyloxy, carbonyloxy, alkylcarbonyloxy, haloakylcarbonyloxy, arylcarbonyloxy, carbonylperoxy, alkylcarbonylperoxy, arylcarbonylperoxy, phosphate, alkylphosphate esters, sulfonic acid, sulfonic alkyl ester, thiosulfate, thiosulfenyl, sulfonamide, —R²³R²⁴ wherein R²³ is S, SS, Po, Po—Po, Se, Se—Se, Te, or Te—Te, and R²⁴ is defined as for R²¹ herein, or Y is

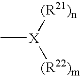

wherein X, R²¹ and R²², are as defined herein.

Moreover, it is contemplated that in some embodiments of the invention, biological matter is provided with a precursor compound that becomes the active version of the Formula I or IV compound by exposure to biological matter, such as by chemical or enzymatic means. In addition, the compound may be provided to the biological matter as a salt of the compound, in the form of a free radical, or a negatively charged, positively charged or multiply charged species. Some compounds qualify as both a Formula I and a Formula II compound and in such cases, the use of the phrase “Formula I or Formula II” is not intended to connote the exclusion of such compounds.

Additional active compounds include, but are not limited to, the following structures, many of which are readily available and known to those of skill in the art (identified by CAS number): 104376-79-6 (Ceftriaxone Sodium Salt); 105879-42-3; 1094-08-2 (Ethopropatine HCl); 1098-60-8 (Triflupromazine HCl); 111974-72-2; 113-59-7; 113-98-4 (Penicillin G K⁺); 115-55-9; 1179-69-7; 118292-40-3; 119478-56-7; 120138-50-3; 121123-17-9; 121249-14-7; 1229-35-2; 1240-15-9; 1257-78-9 (Prochlorperazine Edisylate Salt); 128345-62-0; 130-61-0 (Thioridazine HCl) 132-98-9 (Penicillin V K⁺); 13412-64-1 (Dicloxacillin Na⁺ Hydrate); 134678-17-4; 144604-00-2; 146-54-3; 146-54-5 (Fluphenazine 2HCl); 151767-02-1; 159989-65-8; 16960-16-0 (Adrenocorticotropic Hormone Fragment 1-24); 1982-37-2; 21462-39-5 (Clindamycin HCl); 22189-31-7; 22202-75-1; 23288-49-5 (Probucol); 23325-78-2; 24356-60-3 (Cephapirin); 24729-96-2 (Clindamycin); 25507-04-4; 26605-69-6; 27164-46-1 (Cefazolin Na+); 2746-81-8; 29560-58-8; 2975-34-0; 32672-69-8 (Mesoridazine Benzene Sulfonate); 32887-01-7; 33286-22-5 ((⁺)-cis-Diltiazem HCl); 33564-30-6 (Cefoxitin Na+); 346-18-9; 3485-14-1; 3511-16-8; 37091-65-9 (Azlocillin Na+); 37661-08-8; 3819-00-9; 38821-53-3 (Cephradine); 41372-02-5; 42540-40-9 (Cefamandole Nafate); 4330-99-8 (Trimeprazine hemi-(⁺)-tartrate Salt); 440-17-5 Trifluoperazine 2HCl; 4697-14-7 (Ticarcillin 2Na⁺); 4800-94-6 (Carbenicillin 2Na+); 50-52-2; 50-53-3; 5002-47-1; 51481-61-9 (Cimetidine); 52239-63-1 (6-propyl-2-thiouracil); 53-60-1 (Promazine HCl); 5321-32-4; 54965-21-8 (Albendazole); 5591-45-7 (Thiothixene); 56238-63-2 (Cefuroxime Na⁺); 56796-39-5 (Cefmetazole Na+); 5714-00-1; 58-33-3 (Promethazine HCl); 58-38-8; 58-39-9 (Perphenazine); 58-71-9 Cephalothin Na⁺); 59703-84-3 (Piperacillin Na⁺); 60-99-1 (Methotrimeprazine Maleate Salt); 60925-61-3; 61270-78-8; 6130-64-9 (Penicillin G Procaine Salt Hydrate); 61318-91-0 Sulconazole Nitrate Salt); 61336-70-7 Amoxicillin Trihydrate); 62893-20-3 Cefoperazone Na⁺); 64485-93-4 (Cefotaxime Na+); 64544-07-6; 64872-77-1; 64953-12-4 Moxalactam Na⁺); 66104-23-2 (Pergolide Mesylate Salt); 66309-69-1; 66357-59-3 (Ranitidine HCl); 66592-87-8 (Cefodroxil); 68401-82-1; 69-09-0 (Chlorpromazine HCl); 69-52-3 (Ampicillin Na⁺); 69-53-4 (Ampicillin); 69-57-8 Penicillin G Na⁺); 70059-30-2; 70356-03-5; 7081-40-5; 7081-44-9 (Cloxacillin Na⁺ H₂O); 7177-50-6 Nafcillin Na+H₂O); 7179-49-9; 7240-38-2 (Oxacillin Na H₂O); 7246-14-2; 74356-00-6; 74431-23-5; 74849-93-7; 75738-58-8; 76824-35-6 (Famotidine); 76963-41-2; 79350-37-1; 81129-83-1; 84-02-6 (Prochlorperazine Dimaleate Salt); 87-08-1 (Phenoxymethylpenicillinic Acid); 87239-81-4; 91-33-8 (Benzthiazide); 91832-40-5; 94841-17-5; 99294-94-7; 154-42-7 (6-Thioguanine); 36735-22-5; 536-33-4 (Ethionamide); 52-67-5 (D-Penicillamine); 304-55-2 (Meso-2,3-Dimercaptosuccinic Acid); 59-52-9 2,3-Dimercapto +propanol 6112-76-1 (6-mercaptopurine); 616-91-1 (N-acetyl-L-cysteine); 62571-86-2 (Captopril); 52-01-7 (spironolactone); and, 80474-14-2 (fluticasone propionate).

Selectively targeting mitochondria is considered an embodiment of the invention in some aspects so as to enhance activity of an active compound. Such selective mitochondrial targeting has been accomplished by conjugating agents to a lipophilic triphenylphosphonium cation, which readily cross lipid bilayers and accumulate approximately a 1000 fold within the mitochondrial matrix drive by the large potential (150 to −180 mv) across the mitochondrial inner membrane. Analogs of both vitamin E and ubiquinone have been prepared and used to successfully target mitochondria. (Smith et al., 1999; Kelso et al., 2001; Dhanasekaran et al., 2004). A thiol, thibutyltriphosphonium bromide, has been prepared and used to target mitochondria wherein it accumulated several hundred-fold (Burns et al., 1995; Burns & Murphey), 1997).

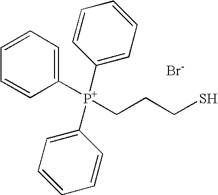

Such conjugates would appear to be suitable candidates for active compounds. In addition to free thiol agents, thiosulfenyl substituted compounds, (H—S—S—R) may be useful. It is contemplated that in some embodiments the agents have the structure:

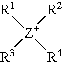

W here Z is P or N;

R¹, R² and R³ are aryl, hetroaryl, akylaryl, cycloalkyl, or alkyl (suitably phenyl, benzyl, tolyl, pyridyl, cyclohexyl, C₃-C₁₀ alkyl, optionally halogenated);

R⁴ is —R⁵SR⁶, wherein R⁵ is C₁-C₁₀ alkyl, R⁶ is H or SH, SO₃H, or PO₃H.

Other active compounds may be identified using a variety of different tests. Reduced metabolism can be measured by a number of ways, including by quantifying the amount of oxygen consumed by a biological sample, the amount of carbon dioxide produced by the sample (indirect measurement of cellular respiration), or characterizing motility.

3. Methods of Use

The present invention provides a variety of methods for enhancing survivability of biological material under ischemic or hypoxic conditions, which involve contacting the biological material with adenosine, an adenosine derivative or analog, an adenosine receptor agonist, a chemical entity that is taken up by a nucleoside transporter, or a chemical entity that binds to and/or modulates a nucleoside transporter. In various embodiments, the biological material is contacted prior to being subjected to ischemic or hypoxic conditions. In other embodiments, the biological material is contacted during all or part of the time of exposure to ischemic or hypoxic conditions. In another related embodiment, the biological material is contacted both prior to and during all or part of the time of exposure to ischemic or hypoxic conditions. It is understood that while specific illustrative embodiments described herein may refer to adenosine, these embodiments may also be practiced using adenosine derivatives and analogs, adenosine receptor agonists, and chemical entities that bind to and/or is taken up by nucleoside transporters.

Enhancing survivability generally refers to either or both of (1) increasing the likelihood that a biological material will survive exposure to ischemic or hypoxic conditions and (2) extending the duration of time that a biological material survives exposure to ischemic or hypoxic conditions. In particular embodiments, by contacting the biological material with adenosine, an adenosine derivative or analog, an adenosine receptor agonist, a chemical entity that is taken up by a nucleoside transporter, or a chemical entity that binds to and/or modulates a nucleoside transporter, the likelihood that the biological material will survive being exposed to hypoxic or ischemic conditions is increased by at least 50%, at least 100%, at least 200%, at least 300%, at least 400%, at least 500%, or at least 1000%. In other embodiments, by contacting the biological material with adenosine, an adenosine derivative or analog, an adenosine receptor agonist, a chemical entity that is taken up by a nucleoside transporter, or a chemical entity that binds to and/or modulates a nucleoside transporter, the duration of time that the biological material will survive during or after exposure to ischemic or hypoxic conditions is increase by at least 50%, at least 100%, at least 200%, at least 300%, at least 400%, at least 500%, or at least 1000%.

It is understood that the particular applications of the methods of the present invention vary depending upon the type of biological material being treated, i.e., cells, tissues, organs, or organisms, and the particular ischemic or hypoxic conditions under which the biological material is exposed. Specific embodiments related to particular types of biological material and ischemic or hypoxic conditions are described further herein. lschemic and hypoxic conditions may be accidental or purposeful, and ischemic and hypoxic conditions may result from a variety of biological and environmental factors. For example, in the context of mammals, ischemic and hypoxic conditions include those resulting from injury or disease, as well as those resulting from cryopreservation techniques. In the context of tissues and organs, hypoxic and ischemic conditions may arise during procedures to preserve organs or tissues prior to transplant or grafting. Similarly, cells may be exposed to hypoxic or ischemic conditions during cryopreservation.

Specific examples of conditions leading to ischemia and hypoxia include, but are not limited to, when oxygen concentrations are reduced in the environment (hypoxia or anoxia, such as at high altitudes or under water); when the biological material is incapable of receiving that oxygen (such as during ischemia), which can be caused by i) reduced blood flow to organs (e.g., heart, brain, and/or kidneys) as a result of blood vessel occlusion (e.g., myocardial infarction, and/or stroke), ii) extracorporeal blood shunting as occurs during heart/lung bypass surgery (e.g., “pumphead syndrome” in which heart or brain tissue is damaged as a result of cardiopulmonary bypass), or iii) as a result of blood loss due to trauma (e.g., hemorrhagic shock or surgery); hypothermia, where the biological material is subjected to sub-physiological temperatures, due to exposure to cold environment or a state of low temperature of the biological material, such that it is unable to maintain adequate oxygenation of the biological materials; hyperthermia, whereby temperatures where the biological material is subjected to supra-physiological temperatures, due to exposure to hot environment or a state of high temperature of the biological material such as by a malignant fever; and conditions of excess heavy met als, such as iron disorders (genetic as well as environmental) such as hemochromatosis, acquired iron overload, sickle-cell anemia, juvenile hemochromatosis African siderosis, thalassemia, porphyria cutanea tarda, sideroblastic anemia, iron-deficiency anemia and anemia of chronic disease.

a. In Vivo methods

In certain embodiments, the present invention provides methods of enhancing the survivability of whole organisms, including, e.g., mammals, that are subjected to ischemic or hypoxic conditions. In related embodiments, the present invention provides methods of preventing injury of whole organisms, including, e.g., mammals, including cell or tissue injuries resulting from ischemia or hypoxia. It is understood that the whole animal or only a portion thereof, e.g., a particular organ, may be subjected to ischemic or hypoxic conditions. However, in particular embodiments, the whole organism may be subjected to ischemic conditions, for example, to assist in the preservation of the organism.

In particular embodiments, the ischemic or hypoxic conditions are the result of an injury or disease suffered by the organism. Accordingly, the present invention provides methods of enhancing survivability of an organism suffering from any disease or injury, including those described below, which methods comprise contacting the organism with an effective amount of adenosine, an adenosine derivative or analog, an adenosine receptor agonist, a chemical entity that is taken up by a nucleoside transporter, or a chemical entity that binds to and/or modulates a nucleoside transporter.

Examples of specific diseases that can induce ischemia or hypoxia include, but are not limited to, tumors, heart diseases, and neurological diseases. Specific injuries that can result in ischemic or hypoxic conditions include, but are not limited to, external insults, such as burns, cutting wounds, amputations, gunshot wounds, or surgical trauma. In addition, injuries include internal insults, such as stroke or heart attack, which result in the acute reduction in circulation. Other injuries include reductions in circulation due to non-invasive stress, such as exposure to cold or radiation, or a planned reduction in circulation, e.g., during heart surgery. On a cellular level, such injuries often result in exposure of cells, tissues, and/or organs to hypoxic conditions, thereby resulting in induction of programmed cell death, or “apoptosis.” Systemically, these injuries can lead to the induction of a series of biochemical processes, such as clotting, inflammation, hypotension, and may give rise to shock, which if it persists may lead to organ dysfunction, irreversible cell damage and death. In a specific scenario, where medical attention is not readily available, such contacting, alternatively in conjunction with reduction in the temperature of the tissue, organ or organism, can “buy time” for the subject, either by bringing medical attention to the subject, or by transporting the subject to the medical attention.

The present invention also contemplates methods for inducing tissue regeneration and wound healing by prevention/delay of biological processes that may result in delayed wound healing and tissue regeneration. In this context, in scenarios in which there is a substantial wound to the limb or organism, the contacting with adenosine, an adenosine derivative or analog, an adenosine receptor agonist, a chemical entity that is taken up by a nucleoside transporter, or a chemical entity that binds to and/or modulates a nucleoside transporter, in vivo or ex vivo, alone or in combination with another active compound or reduced oxygen conditions, alternatively in conjunction with reduction in the temperature of the tissue, organ or organism, aids in the wound healing and tissue regeneration process by managing the biological processes that inhibit healing and regeneration.

In certain embodiments, methods of the present invention can be implemented to enhance survivability and prevent ischemic injury resulting from cardiac arrest or stroke. Accordingly, in one embodiment, the present invention includes methods of enhancing survivability or reducing ischemic injury in a patient suffering from or at risk of cardiac arrest or stroke, comprising providing an effective amount of adenosine, an adenosine derivative or analog, an adenosine receptor agonist, a chemical entity that is taken up by a nucleoside transporter, or a chemical entity that binds to and/or modulates a nucleoside transporter to the patient before, after, or both before and after myocardial infarction, cardiac arrest or stroke.

In certain embodiments, methods of the present invention include pre-treating a biological material, e.g., a patient, prior to an ischemic or hypoxic injury or disease insult. These methods can be used when an injury or disease with the potential to cause ischemia or hypoxia is scheduled or elected in advance, or predicted in advance to likely occur. Examples of such situations include, but are not limited to, major surgery where blood loss may occur spontaneously or as a result of a procedure, cardiopulmonary bypass in which oxygenation of the blood may be compromised or in which vascular delivery of blood may be reduced (as in the setting of coronary artery bypass graft (CABG) surgery), or in the treatment of organ donors prior to removal of donor organs for transport and transplantation into a recipient in need of an organ transplant. Other examples include, but are not limited to, medical conditions in which a risk of injury or disease progression is inherent (e.g., in the context of unstable angina, following angioplasty, bleeding aneurysms, hemorrhagic strokes, following major trauma or blood loss), or in which the risk can be diagnosed using a medical diagnostic test. In one embodiment, the ischemia or hypoxia is not myocardial ischemia or hypoxia. In one embodiment, the ischemia or hypoxia is not due to myocardial infarction. In another embodiment, the biological material is not a myocyte or heart tissue.

In certain embodiments, exposure to adenosine, an adenosine derivative or analog, an adenosine receptor agonist, a chemical entity that is taken up by a nucleoside transporter, or a chemical entity that binds to and/or modulates a nucleoside transporter enhances survivability or reduces damage when exposure occurs before the injurious or disease insult. In other embodiments, exposure to adenosine, an adenosine derivative or analog, an adenosine receptor agonist, a chemical entity that is taken up by a nucleoside transporter, or a chemical entity that binds to and/or modulates a nucleoside transporter, enhances survivability or reduces damage when exposure occurs after the onset or detection of the injurious or disease insult, and either before or after the injury or disease causes ischemia or hypoxia.

In certain embodiments, the present invention includes methods of enhancing survivability of a mammal undergoing a surgery. In a related embodiment, a method is provided for protecting a mammal from suffering ischemic injury or cellular damage resulting from a surgery. These methods comprise providing to the mammal an effective amount of adenosine, an adenosine derivative or analog, an adenosine receptor agonist, a chemical entity that is taken up by a nucleoside transporter, or a chemical entity that binds to and/or modulates a nucleoside transporter, prior to, during, or both prior to and during the surgery. The surgery may be elective, planned, or emergency surgery, such as, e.g., cardiopulmonary surgery. The adenosine, an adenosine derivative or analog, an adenosine receptor agonist, a chemical entity that is taken up by a nucleoside transporter, or a chemical entity that binds to and/or modulates a nucleoside transporter, may be administered by any means available in the art, including, e.g., intraarterially or intraperitoneally.

The invention has particular importance with respect to the risk of ischemic injury from emergency surgical procedures, such as thoractomy, laparotomy, and splenic transection. Therefore, it includes methods of enhancing survivability or reducing or preventing ischemic injury in a patient undergoing an emergency surgery, comprising providing an effective amount of adenosine, an adenosine derivative or analog, an adenosine receptor agonist, a chemical entity that is taken up by a nucleoside transporter, or a chemical entity that binds to and/or modulates a nucleoside transporter, to the patient before, after, or both before and after surgery.

In another embodiment, the present invention includes a method of enhancing survivability of a mammal suffering from a disease or adverse medical condition that causes ischemia or hypoxia within a region of the mammal. A related embodiment includes a method of protecting a mammal from suffering ischemic injury or cellular damage from a disease or adverse medical condition. These methods typically comprise providing to the mammal an effective amount of adenosine, an adenosine derivative or analog, an adenosine receptor agonist, a chemical entity that is taken up by a nucleoside transporter, or a chemical entity that binds to and/or modulates a nucleoside transporter, prior to, after, or both prior to and after, the onset of or progression of the disease or adverse medical condition. This embodiment may be used in the context of a variety of different diseases and adverse medical conditions, including, e.g., unstable angina, post-angioplasty, aneurism, hemorrhagic stroke or shock, trauma, and blood loss.

In specific embodiments, the invention concerns methods of preventing an organism, such as a mammal, from bleeding to death or suffering irreversible tissue damage as a result of bleeding by providing to the mammal an effective amount of adenosine, an adenosine derivative or analog, an adenosine receptor agonist, a chemical entity that is taken up by a nucleoside transporter, or a chemical entity that binds to and/or modulates a nucleoside transporter. In certain additional embodiments, the organism may go into hemorrhagic shock but not die from excessive bleeding. The terms “bleeding” and “hemorrhaging” are used interchangeably to refer to any discharge of blood from a blood vessel. It includes, but is not limited to, internal and external bleeding, bleeding from an injury (which may be from an internal source, or from an external physical source such as from a gunshot, stabbing, physical trauma, etc.).

Moreover, additional embodiments of the invention concern enhancing survivability and preventing irreversible tissue damage from blood loss or other lack of oxygenation to cells or tissue, such as from lack of an adequate blood supply. This may be the result of, for example, actual blood loss, or it may be from conditions or diseases that cause blockage of blood flow to cells or tissue, that reduce blood pressure locally or overall in an organism, that reduce the amount of oxygen is carried in the blood, or that reduces the number of oxygen carrying cells in the blood. Conditions and diseases that may be involved include, but are not limited to, blood clots and embolisms, cysts, growths, tumors, anemia (including sickle cell anemia), hemophilia, other blood clotting diseases (e.g., von Willebrand, ITP), and atherosclerosis. Such conditions and diseases also include those that create essentially hypoxic or anoxic conditions for cells or tissue in an organism because of an injury, disease, or condition.

In one embodiment, the present invention provides methods to enhance the survivability of and prevent injury or damage to biological material undergoing hemorrhagic shock, which include contacting the biological material subjected to shock with adenosine. In certain embodiment, these methods are used to preserve a patient's vital organs and life. Hemorrhagic shock is a life-threatening condition in which adequate perfusion to sustain the physiologic needs of organs or tissues is not present. The resulting inadequate oxygenation of tissues and organs can result in significant tissue and organ damage, and frequently death. Hemorrhagic shock may result from inadequate blood volume (hypovolaemic shock), inadequate cardiac function (cardiogenic shock), or inadequate vasomotor tone, also referred to as distributive shock (neurogenic shock, septic shock, anaphylactic shock). Specific conditions associated with hemorrhagic shock include, e.g., sepsis, blood loss, impaired autoregulation, and loss of autonomic tone, spontaneous hemorrhage (e.g., gastrointestinal bleeding, childbirth), surgery, and other causes. Most frequently, clinical hemorrhagic shock is caused by an acute bleeding episode with a discrete precipitating event. Less commonly, hemorrhagic shock may be seen in chronic conditions with subacute blood loss.

In a particular embodiment, the present invention includes a method of contacting a patient suffering from an acute injury and at risk of or in a state of hemorrhagic shock with an effective amount of adenosine, an adenosine derivative or analog, an adenosine receptor agonist, a chemical entity that is taken up by a nucleoside transporter, or a chemical entity that binds to and/or modulates a nucleoside transporter, within one hour of the injury. This method allows for the patient to be transported to a controlled environment (e.g., surgery), where the initial cause of the shock can be addressed, and then the patient can be brought back to normal function in a controlled manner. For this indication, the first hour after injury, referred to as the “golden hour,” is crucial to a successful outcome. Stabilizing the patient in this time period is the major goal, and transport to a critical care facility (e.g., emergency room, surgery, etc.) where the injury can be properly addressed.

In particular embodiments, the present invention provides methods related to treating cancer and other hyperproliferative diseases. Cancer is a leading cause of mortality in industrialized countries around the world. The most conventional approach to the treatment of cancer is by administering a cytotoxic agent to the cancer patient (or treatment ex vivo of a tissue) such that the agent has a more lethal effect on the cancer cells than normal cells. The higher the dose or the more lethal the agent, the more effective it is in killing cancer cells. However, by the same token, such agents are all that more toxic (and sometimes lethal) to normal cells. Hence, chemo-and radiotherapy are often characterized by severe side effects, some of which are life threatening, e.g., sores in the mouth, difficulty swallowing, dry mouth, nausea, diarrhea, vomiting, fatigue, bleeding, hair loss and infection, skin irritation and loss of energy (Curran, 1998; Brizel, 1998).

In one embodiment, the present invention contemplates the use of adenosine, an adenosine derivative or analog, an adenosine receptor agonist, a chemical entity that is taken up by a nucleoside transporter, or a chemical entity that binds to and/or modulates a nucleoside transporter, to protect normal tissues of a patient being treated for cancer or another hyperproliferative disease, thereby reducing the potential impact of chemo- or radiotherapy on those tissues, and enhancing survivability of the patient. These methods permit the use of higher doses of chemo-and radiotherapy, thereby increasing the anti-cancer effects of these treatments. Recent studies suggest that transient and reversible lowering of the core body temperature, or “hypothermia,” may lead to improvements in the fight against cancer. Hypothermia of 28° C. was recently found to reduce radiation, doxorubicin-and cisplatin-induced toxicity in mice. The cancer fighting activity of these drugs/treatments was not compromised when administered to cooled animals; rather, it was enhanced, particularly for cisplatin (Lundgren-Eriksson et al., 2001).

Treatment of virtually any hyperproliferative disorder, including benign and malignant neoplasias, non-neoplastic hyperproliferative conditions, pre-neoplastic conditions, and precancerous lesions, is contemplated. Such disorders include restenosis, cancer, multi-drug resistant cancer, primary psoriasis and metastatic tumors, angiogenesis, rheumatoid arthritis, inflammatory bowel disease, psoriasis, eczema, and secondary cataracts, as well as oral hairy leukoplasia, bronchial dysplasia, carcinomas in situ, and intraepithelial hyperplasia. In particular, the present invention is directed at the treatment of human cancers including cancers of the prostate, lung, brain, skin, liver, breast, lymphoid system, stomach, testicles, ovaries, pancreas, bone, bone marrow, gastrointestine, head and neck, cervix, esophagus, eye, gall bladder, kidney, adrenal glands, heart, colon and blood. Cancers involving epithelial and endothelial cells are also contemplated for treatment.

In certain embodiments, adenosine, an adenosine derivative or analog, an adenosine receptor agonist, a chemical entity that is taken up by a nucleoside transporter, or a chemical entity that binds to and/or modulates a nucleoside transporter, is provided to a patient suffering from cancer or another hyperproliferative disease or condition in combination with one or more anti-proliferative agents effective in the treatment of hyperproliferative disease. An antiproliferative agent is an agent capable of negatively affecting cell growth in a subject, for example, by killing cancer cells, inducing apoptosis in cancer cells, reducing the growth rate of cancer cells, reducing the incidence or number of metastases, reducing tumor size, inhibiting tumor growth, reducing the blood supply to a tumor or cancer cells, promoting an immune response against cancer cells or a tumor, preventing or inhibiting the progression of cancer, or increasing the lifespan of a subject with cancer.

In specific embodiments of the present invention, a patient suffering from cancer or another hyperproliferative disease is contacted with adenosine, an adenosine derivative or analog, an adenosine receptor agonist, a chemical entity that is taken up by a nucleoside transporter, or a chemical entity that binds to and/or modulates a nucleoside transporter, in combination with one or more anticancer agents. In particular embodiments, the adenosine or other chemical entity enhances survivability of the patient suffering from cancer or other hyperproliferative disease, while in other embodiments, the adenosine or other chemical entity protects the patient from ischemic injury caused by the one or more anticancer agents. In particular embodiments, the use of adenosine or other chemical entity allows the patient to be exposed to greater amounts of the anticancer treatment, so one embodiment includes contacting a patient with adenosine in combination with a higher dose of anticancer agent or a longer duration of contact with the anticancer agent as compared to the amount routinely used or considered safe in the absence of adenosine.

Anticancer agents include, but are not limited to, biological agents (biotherapy), chemotherapy agents, and radiotherapy agents. In one embodiment, methods of the present invention involve contacting or exposing a patient (or their cells) with adenosine and the anticancer agent(s) at the same time. This may be achieved by contacting the cell with a single composition or pharmacological formulation that includes both agents, or by contacting or exposing the cell with two distinct compositions or formulations, at the same time, wherein one composition includes adenosine and the other includes the anticancer agent(s). Alternatively, treatment with adenosine may precede or follow the anticancer agent treatment by intervals ranging from minutes to weeks. In embodiments where the adenosine and anticancer agent are applied separately to the cell, one would generally ensure that a significant period of time did not expire between the time of each delivery, such that the adenosine and secondary agent would still be able to exert an advantageously combined effect on the cell. In such instances, it is contemplated that one may contact the cell with both modalities within about 12-24 h of each other and, more preferably, within about 6-12 h of each other. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations. In some embodiments of the invention, biological matter is exposed to adenosine for about, at least, at least about, or at most about 30 seconds, 1, 2, 3, 4, 5, 10, 15, 20, 25, 30, 35, 40, 45, 50, 55 minutes, 1, 2, 3, 4, 5, 6 hours or more, and any range or combination therein.

Administration of the adenosine and chemotherapeutics to a patient will follow general protocols for the administration of chemotherapeutics, taking into account the toxicity, if any, of the compound. It is expected that the treatment cycles would be repeated as necessary. It also is contemplated that various standard therapies, as well as surgical intervention, may be applied in combination with the above-described anti-cancer therapy. It is further contemplated that any combination treatment contemplated for use with adenosine and a non-active compound (such as chemotherapy), may be applied with respect to adenosine or multiple active compounds.

Chemotherapeutic agents that may be used in combination with adenosine according to methods of the present invention include, for example, cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, taxol, gemcitabien, navelbine, farnesyl-protein transferase inhibitors, transplatinum, 5-fluorouracil, vincristine, vinblastine and methotrexate, Temazolomide (an aqueous form of DTIC), or any analog or derivative variant of the foregoing.

The methods of the present invention may also be practiced using a combination of adenosine, an adenosine derivative or analog, an adenosine receptor agonist, a chemical entity that is taken up by a nucleoside transporter, or a chemical entity that binds to and/or modulates a nucleoside transporter, and radiotherapy. Examples of radiotherapy that have been used extensively include what are commonly known as γ-rays, X-rays, and/or the directed delivery of radioisotopes to tumor cells. Other forms of DNA damaging factors are also contemplated such as microwaves and UV-irradiation. It is most likely that all of these factors affect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

Methods of the invention further include contacting a patient with adenosine, an adenosine derivative or analog, an adenosine receptor agonist, a chemical entity that is taken up by a nucleoside transporter, or a chemical entity that binds to and/or modulates a nucleoside transporter, in combination with an immunotherapeutic agent. Immunotherapeutics, generally, rely on the use of immune effector cells and molecules to target and destroy cancer cells. The immune effector may be, for example, an antibody specific for some marker on the surface of a tumor cell. The antibody alone may serve as an effector of therapy or it may recruit other cells to actually effect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells. In one aspect of immunotherapy, the tumor cell bears some marker that is amenable to targeting, i.e., is not present on the majority of other cells. Many tumor markers exist and any of these may be suitable for targeting in the context of the present invention. Common tumor markers include carcinoembryonic antigen, prostate specific antigen, urinary tumor associated antigen, fet al antigen, tyrosinase (p97), gp68, TAG-72,HMFG, Sialyl Lewis Antigen, MucA, MucB, PLAP, estrogen receptor, laminin receptor, erb B and p155. An alternative aspect of immunotherapy is to anticancer effects with immune stimulatory effects. Immune stimulating molecules also exist including: cytokines such as IL-2, IL-4, IL-12, GM-CSF, gamma-IFN, chemokines such as MIP-1, MCP-1, IL-8 and growth factors such as FLT3 ligand. Combining immune stimulating molecules, either as proteins or using gene delivery in combination with a tumor suppressor such as mda-7 has been shown to enhance anti-tumor effects (Ju et al., 2000)

Immunotherapies contemplated by the present invention also include, but are not limited to, immune adjuvants (e.g., Mycobacterium bovis, Plasmodium falciparum, dinitrochlorobenzene and aromatic compounds) (U.S. Pat. No. 5,801,005; U.S. Pat. No. 5,739,169;Hui and Hashimoto, 1998; Christodoulides et al., 1998 ), cytokine therapy (e.g., interferons α, β and γ; IL-1, GM-CSF and TNF) (Bukowski et al., 1998; Davidson et al., 1998;Hellstrand et al., 1998) gene therapy (e.g., TNF, IL-1, IL-2, p53) (Qin et al., 1998; Austin-Ward and Villaseca, 1998; U.S. Pat. No. 5,830,880 and U.S. Pat. No. 5,846,945) and monoclonal antibodies (e.g., anti-ganglioside GM2, anti-HER-2, anti-p185) (Pietras et al., 1998;Hanibuchi et al., 1998). Herceptin (trastuzumab) is a chimeric (mouse-human) monoclonal antibody that blocks the HER2-neu receptor. It possesses anti-tumor activity and has been approved for use in the treatment of malignant tumors (Dillman, 1999). Combination therapy of cancer with herceptin and chemotherapy has been shown to be more effective than the individual therapies. Thus, it is contemplated that one or more anti-cancer therapies may be employed with the anti-tumor therapies described herein.

The methods of the present invention may be used in the treatment of neurodegenerative diseases associated with ischemia or hypoxia. Neurodegenerative diseases are characterized by degeneration of neuronal tissue, and are often accompanied by loss of memory, loss of motor function, and dementia. With dementing diseases, intellectual and higher integrative cognitive faculties become more and more impaired over time. It is estimated that approximately 15% of people 65 years or older are mildly to moderately demented. Neurodegenerative diseases include Parkinson's disease; primary neurodegenerative disease; Huntington's Chorea; stroke and other hypoxic or ischemic processes; neurotrauma; metabolically induced neurological damage; sequelae from cerebral seizures; hemorrhagic shock; secondary neurodegenerative disease (metabolic or toxic); Alzheimer's disease, other memory disorders; or vascular dementia, multi-infarct dementia, Lewy body dementia, or neurodegenerative dementia. The present invention provides methods of preventing tissue damage from neurological diseases associated with ischemia, comprising administering adenosine, an adenosine derivative or analog, an adenosine receptor agonist, a chemical entity that is taken up by a nucleoside transporter, or a chemical entity that binds to and/or modulates a nucleoside transporter, to a patient suffering from such a disease or condition.

In yet another embodiment, the methods of the present invention are used to treat a mammal with extreme hypothermia. The methods and compositions of the present invention are useful for enhancing survivability of an organism subjected to extreme hypothermia. In one embodiment, these methods include enhancing survivability of an organism by inducing mild hypothermia in the organism in combination with contacting the organism with adenosine. Hypothermia can be mild, moderate or profound. Mild hypothermia comprises achievement of a core body temperature of approximately between 0.1 and 5 degrees Celsius below the normal core body temperature of the mammal. The normal core body temperature of a mammal is usually between 35 and 38 degrees Celsius. Moderate hypothermia comprises achievement of a core body temperature of approximately between 5 and 15 degrees Celsius below the normal core body temperature of the mammal. Profound hypothermia comprises achievement of a core body temperature of approximately between 15 and 37 degrees Celsius below the normal core body temperature of the mammal.

Mild hypothermia is known in the art to be therapeutically useful and effective in both non-human mammals and in humans. The therapeutic benefit of mild hypothermia has been observed in human clinical trials in the context of out-of-hospital cardiac arrest. Exposure of humans to mild hypothermia in the context of cardiac arrest results in a survival advantage and an improved neurological outcome compared to standard of care with normothermia, or absence of mild hypothermia (Bernard et al., 2002; The Hypothermia After Cardiac Arrest Study Group et al. 2002).

In one embodiment, a method of the present invention provides that patients with extreme hypothermia are administered or exposed to adenosine, an adenosine derivative or analog, an adenosine receptor agonist, a chemical entity that is taken up by a nucleoside transporter, or a chemical entity that binds to and/or modulates a nucleoside transporter, and then gradually restored to normal temperature while withdrawing, in a controlled fashion, the adenosine. In this way, the adenosine or other chemical entity buffers the biological systems within the subject so that they may be initiated gradually without shock (or harm) to the subject. Ideally, the patient will be stabilized in terms of heart rate, respiration and temperature prior to effecting any change. Once stable, the ambient environmental temperature will be increased, again gradually. This may be accomplished simply by removing the subject from the hypothermic conditions. A more regulated increase in temperature may be effected by adding successive layers of clothing or blankets, by use of a thermal wrap with gradual increase in heat, or if possible, by placing the subject in chamber whose temperature may be gradually increased.

It is preferred that the vital signs of the subject are monitored over the course of the temperature increase. Also, in conjunction with increasing the temperature, the adenosine or other chemical entity is removed from the subject's environment. Both heat and adenosine treatment are ceased at the appropriate endpoint, judged by the medical personnel monitoring the situation, but in any event at the time the subject's temperature and other vital signs return to a normal range. Continued monitoring following cessation of treatment is recommended for a period of at least 24 hrs.

Methods and compositions of the present invention have advantages over other methods known in the art, including, but not limited to, packing the subject in ice, or surrounding the subject with a “cooling tent” that circulates cool air or liquid, for inducing mild, moderate, or profound hypothermia in mammals or humans. In these cases, the subject resists the reduction of core body temperature below normothermia and tries to generate heat by shivering. Shivering, and the body heat engendered therein, can have a negative impact on the achievement of mild hypothermia by, for example, slowing the rate of decrease in the core body temperature that is achieved using the standard methods of hypothermia induction. Consequently, humans subjected to therapeutic levels of hypothermia are also treated with a drug that inhibits shivering (by blocking neurotransmission at the neuromuscular junctions) (Bernard et al., 2002).

In certain embodiments, methods and compositions of the present invention are combined with invasive methods or medical devices known in the art to induce therapeutic hypothermia in mammals or humans. Such invasive methods and devices include, but are not limited to, flexible probes or catheters that can be inserted into the vasculature of the subject in need of hypothermia, wherein the temperature of the catheter is adjusted to below the normal body temperature of the subject, resulting in the cooling of blood which is in contact with the catheter. The cooled blood subsequently engenders a decrease in the core body temperature of the mammal. By incorporating feedback from a thermocouple monitoring the core body temperature of the mammal, the temperature of the catheter can be modulated so as to maintain a pre-specified core body temperature. Such medical devices for achieving and maintaining mild or moderate hypothermia, referred to in the art as endovascular temperature therapy, are known in the art and are described for example on the World Wide Web at innercoool.com and radiantmedical.com.

In other embodiments, the methods of the present invention are used to treat hyperthermia. Under certain conditions, which can result from genetic, infectious, drug, or environmental causes, patients can loose homeostatic temperature regulation resulting in severe uncontrollable fever (hyperthermia). This can result in mortality or long-term morbidity, especially brain damage, if it is not controlled properly. The present invention provides methods of treating hyperthermia that involve contacting the patient with adenosine, an adenosine derivative or analog, an adenosine receptor agonist, a chemical entity that is taken up by a nucleoside transporter, or a chemical entity that binds to and/or modulates a nucleoside transporter (alone or in combination with another active compound) to induce reduced metabolic activity and enhance survivability or reduce injury to potentially affected brain tissue. In particular embodiments, the patient is contacted for between about 6 and about 24 hours, during which time the source of the fever can be addressed. This treatment can be combined with whole-body temperature regulation, such as ice bath/blanket/cooling system.

b. Ex Vivo methods

In certain embodiments, the methods of the present invention are used to enhance the survivability of ex vivo biological matter subjected to hypoxic or ischemic conditions, including, e.g., isolated cells, tissues and organs. Specific examples of such ex vivo biological material include platelets and other blood products, as well as tissues and organs to be transplanted.

In one embodiment, methods of the present invention may be used to enhance survivability of biological material in the laboratory or research context, for example when cell lines or laboratory organisms are purposefully subjected to hypoxic or ischemic conditions, e.g., during cryopreservation and storage.

The present invention can be extended to protecting cells in culture, which might otherwise die or be induced into apoptosis. According to the present invention, cells are exposed to adenosine, an adenosine derivative or analog, an adenosine receptor agonist, a chemical entity that is taken up by a nucleoside transporter, or a chemical entity that binds to and/or modulates a nucleoside transporter, prior to and/or while in culture. Cells that can be cultured according to the invention include those that can eventually be placed back into a physiological context, i.e., those for subsequent transplant. Such cells include, but are not limited to, bone marrow cells, skin cells, stem cells, and epithelial cells. Also, some transplantable cells would greatly benefit from expansion in culture, thereby increasing the amount of material that can be introduced into the host. In one particular embodiment, the methods of the present invention are applied to epithelial cells from the gastrointestinal tract.

Furthermore, the invention extends to the culture of tumor cells. Culture of tumor cells is known to result in alteration of the phenotype and, in some cases, death. This makes tissue culture experiments on tumor cells highly unpredictable.

General cell culture techniques are well known to those of skill in the art. Examples of this knowledge can be found in Shaw (1996) and Davis (1994), both of which are incorporated by reference herein. General information and modifications of traditional cell culture techniques is also found in U.S. Pat. No. 5,580,781, which is incorporated by reference. Furthermore, techniques for culturing skin cells are described in U.S. Pat. No. 6,057,148, which is incorporated by reference. It is contemplated that these techniques, as well as others known to those of skill in the art, will be supplemented with media containing adenosine, an adenosine derivative or analog, an adenosine receptor agonist, a chemical entity that is taken up by a nucleoside transporter, or a chemical entity that binds to and/or modulates a nucleoside transporter.

The invention also provides methods of enhancing the survivability or preserving tissues and organs for transplant, which comprise contacting the tissue or organ with adenosine, an adenosine derivative or analog, an adenosine receptor agonist, a chemical entity that is taken up by a nucleoside transporter, or a chemical entity that binds to and/or modulates a nucleoside transporter. Initial contact can occur prior to removal from a donor or following removal from a donor. While there is a constant need for organ donors, a significant hurdle in providing those in need of an organ transplant with an organ is the limitations in current organ preservation techniques. Indeed, the primary cause of organ transplant failure for transplanted hearts in the first 30 days is ischemic-reperfusion injury. It is widely believed that a human heart must be transported within four hours for there to be any chance of the subsequent transplantation to be a success. Similarly, the maximum cold ischemic time allowed for liver is 12-24 hours, kidney is 48-72 hours, pancreas is 12-24 hours, and small intestine is 12 hours (Rager, 2004). Tissues useful for transplant include, but are not limited to, skin tissue. Organs useful for transplant include, but are not limited to, hearts, lungs, kidneys, livers, pancreas, small intestine, and cornea.

Currently, preserving solid organs depends on rapid intravascular cooling done in situ, followed by removal of the organs, storage of the organs in ice-cold preservation fluid and rapid transport to the recipients' hospitals. The cold ischemic time is the length of time the organs are on ice, without blood flow. The maximum cold ischemic time limits the amount of time that can pass between organ recovery and the organ transplant. Between 2%-10% of matched and procured organs cannot be used due to extended ischemic time, depending on the type of organ. Similarly, approximately 10 to 20% of procured organs are not used due to poor organ function and/or infection (not including HIV/CMV/hepatitis).

Current preservation techniques involve the use of ice-cold solutions that include electrolytes, antioxidants, hydrogen ion buffers and sugars. Punch et a/., 2001. Appropriate tissue matching depends on blood group matching (e.g., blood type, A, B or O) for all organs. Immunosuppresive regimens typically include three drugs: a glucocorticoid such as prednisone, an antimetabolite such as azathiprine or mycophenolate, and a calcineurin inhibitor such as cyclosporine or tacrolimus.

The two most frequently used methods for preserving/transporting hearts for transplantation are hypothermic storage and continuous perfusion. In the former method, the heart is arrested, removed from the donor, and then rapidly cooled and transported in cold storage. In the latter method, the following steps are typically employed: 1) pulsatile flow; 2) hypothermia; 3) membrane oxygenation, and 4) a perfusate containing both.

The methods of the present invention may be used to increase the survivability of donor tissues and organs, thereby extending the time before the donor tissue must be transplanted into a recipient and blood flow restored. These methods may be combined with current preservation methods, including the use of preservation agents and oxygen perfusion. A variety of preservation solutions have been disclosed in which the organ is surrounded or perfused with the preservation solution while it is transported. One of the most commonly used solution is ViaSpan® (Belzer UW), which can be combined with cold storage. Other examples of such solutions or components of such solutions include the St. Thomas solution (Ledingham et al., J. Thorac. Cardiobasc. Surg. 93:240-246, 1987), Broussais solution, UW solution (Ledingham et al., Circulation 82 (Part 2)IV351-8, 1990), Celsior solution (Menasche et al., Eur. J. Cardio. Thorax. Surg. 8:207-213, 1994), Stanford University solution, and solution B20 (Bernard et al., J. Thorac. Cardiovasc. Surg. 90:235-242, 1985), as well as those described and/or claimed in U.S. Pat. Nos. 6,524,785; 6,492,103; 6,365,338; 6,054,261; 5,719,174; 5,693,462; 5,599,659; 5,552,267; 5,405,742; 5,370,989; 5,066,578; 4,938,961; and, 4,798,824. In addition to solutions, other types of materials are also known for use in transporting organs and tissue. These include gelatinous or other semi-solid material, such as those described, for example, in U.S. Pat. No. 5,736,397.

Some of the systems and solutions for organ preservation specifically involve oxygen perfusion in the solution or system to expose the organ to oxygen, because it is believed that maintaining the organ or tissue in an oxygenated environment improves viability. See Kuroda et al., (Transplantation 46(3):457-460, 1988) and U.S. Pat. Nos. 6,490,880; 6,046,046; 5,476,763; 5,285,657; 3,995,444; 3,881,990; and, 3,777,507. A variety of systems and containers for transporting organs and tissues have been developed, which provide cooling and/or oxygen perfusion. These may be employed in combination with contacting the tissue or organ with adenosine, according to the present invention. Specific apparatuses include, for example, cooling systems described in U.S. Pat. Nos. 4,292,817, 4,473,637, 4,745,759, 5,434,045 and 4,723,974. Others constitute a system in which an apparatus is devised for perfusion of the organ or tissue in a preservation solution, as is described in U.S. Pat. Nos. 6,490,880; 6,100,082; 6,046,046; 5,326,706; 5,285,657; 5,157,930; 4,951,482; 4,502,295; and, 4,186,565.

In certain embodiments, the present invention provides methods to enhance survivability of platelets. Platelets are small cell fragments (˜⅓ size of erythrocytes) that play a vital role in the formation of blood clots at the site of bleeding. Platelet concentrates are transfused for a variety of indications, for example: 1) to prevent bleeding due to thrombocytopenia; 2) in a bleeding patient to maintain a platelet count above 50,000; 3) to address abnormal platelet function that is congenital or due to medications, sepsis, malignancy, tissue trauma, obstetrical complications, extra corporeal circulation, or organ failure such as liver or kidney disease.

Each unit of platelets contains an average of 0.8-0.85×10¹¹ platelets. Platelet concentrates also contain about 60 mL of plasma (coagulation factors) and small numbers of red blood cells and leukocytes. Platelet units must be maintained at room temperature (20° C.-24° C.) and agitated during storage. They can be stored at the Blood Center for up to 5 days. Longer storage is not possible at present due to deterioration of the platelets, and the risk of microbial contamination. Two sources of platelets currently exist: (1) pooled random donor platelet concentrates prepared from platelets that have been harvested by centrifuging units of whole blood; and (2) apheresis platelets, collected from a single donor, prepared in standard (equivalent to ˜4 pooled units) and “large” (equivalent to ˜6 pooled units) sizes.

Platelet storage poses problems that are not found with the storage of whole blood or other components. While whole blood, red and white cells may be stored at 4° C. for weeks, platelets will aggregate in cold storage and when allowed to settle. Therefore, the standard method of storing platelets is at room temperature, approximately 20 to 24° C., with gentle agitation. Even under these conditions, platelets can only be stored for 5 days before they need to be discarded. This problem of outdating results in approximately $500 million annually in lost revenue for US hospitals. If even a moderate increase in shelf life could be attained, approximately 90% of this loss could be avoided.

An additional problem with platelet storage is bacterial contamination. Contamination is primarily due to staphylococci from the skin during the phlebotomy, or else donor bacteremia. The bacterial contamination of platelets represents the largest infectious risk with any blood transfusion procedure.

A significant factor affecting the viability of platelets is regulation of pH. Virtually all units of platelets stored according to the currently accepted methods show a decrease in pH from their initial value of approximately 7.0. This decrease is primarily due to the production of lactic acid by platelet glycolysis and to a lesser extent to accumulation of CO₂ from oxidative phosphorylation. As the pH falls, the platelets change shape from discs to spheres. If the pH falls below 6.0, irreversible changes in platelet morphology and physiology render them non-viable after transfusion. An important goal in platelet preservation, therefore, is to prevent this decrease in pH. It was previously thought that platelets must be stored in a container permeable to oxygen since glycolysis is stimulated when oxygen availability is limited (see e.g., U.S. Pat. No. 5,569,579). However, it has more recently been demonstrated that the viability of stored platelets can be extended by storing them in an anoxic environment.

The present invention provides methods of enhancing survivability of platelets, including, in particular embodiments, platelets stored in an anoxic environment, comprising contacting the platelets with an effective amount of adenosine, an adenosine derivative or analog, an adenosine receptor agonist, a chemical entity that is taken up by a nucleoside transporter, or a chemical entity that binds to and/or modulates a nucleoside transporter, during storage.

In various embodiments of the methods of the present invention, including those specifically exemplified above, biological material is exposed to adenosine, an adenosine derivative or analog, an adenosine receptor agonist, a chemical entity that is taken up by a nucleoside transporter, or a chemical entity that binds to and/or modulates a nucleoside transporter, once or more than one time. In certain embodiments, biological matter is exposed to adenosine 1, 2, 3, 4, 5, 6, 7, 8, 9, 10 or more times, meaning when a biological matter is exposed multiple times that there are periods of respite (with respect to exposure to the active compound) in between.

It is also contemplated that adenosine, an adenosine derivative or analog, an adenosine receptor agonist, a chemical entity that is taken up by a nucleoside transporter, or a chemical entity that binds to and/or modulates a nucleoside transporter, may be administered before, during, after, or any combination thereof, in relation to the onset or progression of an injurious insult or disease condition. In certain embodiments, pre-treatment of biological matter with adenosine or other chemical entity and/or one or more active compounds is sufficient to enhance survivability and/or reduce damage from an injurious or disease insult. Pre-treatment is defined as exposure of the biological matter to adenosine before the onset or detection of the injurious or disease insult. Pre-treatment can be followed by termination of exposure at or near the onset of the insult or continued exposure after the onset of insult.

In various embodiments, the present invention comprises contacting living biological matter with an effective amount of adenosine, an adenosine derivative or analog, an adenosine receptor agonist, a chemical entity that is taken up by a nucleoside transporter, or a chemical entity that binds to and/or modulates a nucleoside transporter. The term “effective amount” means an amount that can achieve the stated result. In certain methods of the present invention, an “effective amount” is, for example, an amount that enhances the survivability of biological matter in response to ischemic or hypoxic conditions, or an amount that protects biological material from injury due to ischemic or hypoxic conditions.

In some embodiments, an effective amount is characterized as a sublethal dose. In the context of cells, tissues, or organs (not the whole organism), a “sublethal dose” means a single administration that is less than half of the amount that would cause at least a majority of cells in a biological matter to die within 24 hours of the administration. In the context of the entire organism, then a “sublethal dose” means a single administration that is less than half of the amount that would cause the organism to die within 24 hours of the administration. In other embodiments, an effective amount is characterized as a near-lethal dose. Similarly, in the context of cells, tissues, or organs (not the whole organism), a “near lethal dose” means a single administration that is within 25% of the amount that would cause at least a majority of cell(s) to die within 24 hours of the administration. If treatment of the entire organism is desired, then a “near lethal dose” means a single administration that is within 25% of the amount that would cause the organism to die within 24 hours of the administration. In some embodiments a sublethal dose is administered by administering a predetermined amount of adenosine, an adenosine derivative or analog, an adenosine receptor agonist, a chemical entity that is taken up by a nucleoside transporter, or a chemical entity that binds to and/or modulates a nucleoside transporter, or active compound to the biological material.

Furthermore, it is contemplated that in some embodiments an effective amount is characterized as a supralethal dose of adenosine, an adenosine derivative or analog, an adenosine receptor agonist, a chemical entity that is taken up by a nucleoside transporter, or a chemical entity that binds to and/or modulates a nucleoside transporter, or active compound. In the context of cells, tissues, or organs (not the whole organism), a “supra-lethal dose” means a single administration that is at least 1.5 times (1.5×) the amount that would cause at least a majority of cells in a biological matter to die within 24 hours of the administration. If treatment of the entire organism is desired, then a “supra-lethal dose” means a single administration that is at least 1.5 times the amount that would cause the organism to die within 24 hours of the administration. It is specifically contemplated that the supra-lethal dose can be about, at least about, or at most about 1.5×, 2×, 3×, 4×, 5×, 10×, 20×, 30×, 40×, 50×, 60×, 70×, 80×, 90×, 100×, 150×, 200×, 250×, 300×, 400×, 500×, 600×, 700×, 800×, 900×, 1000×, 1100×, 1200×, 1300×, 1400×, 1500×, 1600×, 1700×, 1800×, 1900×, 2000×, 3000×, 4000×, 5000×, 6000×, 7000×, 8000×, 9000×, 10,000× or more, or any range derivable therein, the amount that would cause at least a majority of cells in a biological matter (or the entire organism) to die within 24 hours of the administration.

The amount adenosine, an adenosine derivative or analog, an adenosine receptor agonist, a chemical entity that is taken up by a nucleoside transporter, or a chemical entity that binds to and/or modulates a nucleoside transporter, or active compound that is provided to biological material can be about, at least, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 mg, mg/kg, or mg/m2, or any range derivable therein. Alternatively, the amount may be expressed as 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 mM or M, or any range derivable therein.

In various embodiments of the present invention, biological material is exposed to adenosine, an adenosine derivative or analog, an adenosine receptor agonist, a chemical entity that is taken up by a nucleoside transporter, or a chemical entity that binds to and/or modulates a nucleoside transporter, for about, at least, at least about, or at most about 30 seconds, 1, 2, 3, 4, 5, 10, 15, 20, 25, 30, 35, 40, 45, 50, 55 minutes, 1, 2, 3, 4, 5, 6, 7, 8, 9, 10, 11, 12, 13, 14, 15, 16, 17, 18, 19, 20, 21, 22, 23, 24 hours, 1, 2, 3, 4, 5, 6, 7 days or more, and any range or combination therein.

In some embodiments, an effective amount is administered by monitoring, alone or in combination, the amount of adenosine, an adenosine derivative or analog, an adenosine receptor agonist, a chemical entity that is taken up by a nucleoside transporter, or a chemical entity that binds to and/or modulates a nucleoside transporter, and/or active compound administered, monitoring the duration of administration, monitoring a physiological response (e.g., pulse, respiration, pain response, movement or motility, metabolic parameters such as cellular energy production or redox state, etc.) of the biological material to the administration of the adenosine, an adenosine derivative or analog, an adenosine receptor agonist, a chemical entity that is taken up by a nucleoside transporter, or a chemical entity that binds to and/or modulates a nucleoside transporter, or active compound, and reducing, interrupting or ceasing administration of the adenosine and/or active compound when a predetermined floor or ceiling for a change in that response is measured. Moreover, these steps can be employed additionally in any method of the invention.

The term “expose” is used according to its ordinary meaning to indicate that biological matter is subjected to adenosine and/or an active compound. In certain embodiments, this is achieved by contacting the biological matter with adenosine or an active compound. In the case of in vivo cells, tissues, or organs, “expose” may further mean “to lay open” these materials so that it can be contacted with an adenosine or active compound. This can be done, for example, surgically. Exposing biological matter to adenosine or active compound can be by incubation in or with (includes immersion), perfusion or infusion, injection of biological matter, or applying adenosine or active compound to the biological matter. In addition, if treatment of an entire organism is desirable, inhalation or ingestion of the adenosine or active compound, or any route of systemic administration is contemplated. Furthermore, the term “provide” is used according to its ordinary and plain meaning to mean “to supply.” It is contemplated that adenosine or an active compound may be provided to biological matter in one form and be converted by chemical reaction to its form as an active compound. The term “provide” encompasses the term “expose” in the context of the term “effective amount,” according to the present invention.

The present invention also provides methods and compositions for preserving both non-living biological material and preserving or extending the shelf-life of non-biological material. These methods comprise contacting the non-living biological material or non-biological material with adenosine, an adenosine derivative or analog, an adenosine receptor agonist, a chemical entity that is taken up by a nucleoside transporter, or a chemical entity that binds to and/or modulates a nucleoside transporter, or an active compound. In certain embodiments, the non-living biological material or non-biological material is contacted with adenosine, an adenosine derivative or analog, an adenosine receptor agonist, a chemical entity that is taken up by a nucleoside transporter, or a chemical entity that binds to and/or modulates a nucleoside transporter, or an active compound prior to storage, while in other embodiments, the non-living biological material is contacted with adenosine, an adenosine derivative or analog, an adenosine receptor agonist, a chemical entity that is taken up by a nucleoside transporter, or a chemical entity that binds to and/or modulates a nucleoside transporter, or an active compound during storage. In these embodiments of the present invention, an effective amount is considered an amount sufficient to preserve a non-living biological material for a duration of time at least 50% or at least 100% longer than in the absence of contacting the non-living biological material with an active compound. An effective amount may also be defined as an amount sufficient to reduce the degradation rate of a non-living biological material by at least 25% or 50% as compared to the rate of degradation of the biological material when not contacted with adenosine, an adenosine derivative or analog, an adenosine receptor agonist, a chemical entity that is taken up by a nucleoside transporter, or a chemical entity that binds to and/or modulates a nucleoside transporter, or an active compound. An effective amount is further defined as an amount sufficient to extend the shelf-life of a product, e.g., food, beverage, pharmaceutical, health care, or cosmetic, by at least 50% or at least 100% as compared to the shelf-life of the product when not contacted with adenosine, an adenosine derivative or analog, an adenosine receptor agonist, a chemical entity that is taken up by a nucleoside transporter, or a chemical entity that binds to and/or modulates a nucleoside transporter, or an active compound.

Methods of the present invention may be combined with other preservation methods available in the art, including methods such as storing a non-living biological material or non-biological material at low temperature conditions, contacting the non-living biological material or non-biological material with preservatives or embalming fluids, and contacting the non-living biological material or non-biological material with antibacterial agents.

In the context of non-living biological material, the methods of the present invention are useful in a variety of contexts. For example, they may be used to preserve a dead animal, e.g., a cadaver, prior to burial, cremation, dissection, or taxidermy. The present invention, therefore, provides a method of preserving a dead animal comprising contacting the dead animal with an active compound. This method may be accomplished in a variety of manners, including, e.g., adding an active compound to embalming fluid, immersing the dead animal in a solution comprising an active compound, or infusing the dead animal with a solution comprising adenosine, an adenosine derivative or analog, an adenosine receptor agonist, a chemical entity that is taken up by a nucleoside transporter, or a chemical entity that binds to and/or modulates a nucleoside transporter, or an active compound.

These methods may also be used to preserve non-living biological material for scientific or investigative purposes, e.g., when the non-living biological material is a biological sample to be tested for disease or infection or used to obtain blood type or DNA sequence information, e.g., for forensic or identification purposes. Accordingly, in one embodiment, the present invention provides a method of preserving a non-living biological sample comprising contacting the sample with or storing the sample in the presence of adenosine, an adenosine derivative or analog, an adenosine receptor agonist, a chemical entity that is taken up by a nucleoside transporter, or a chemical entity that binds to and/or modulates a nucleoside transporter, or an active compound. The invention further provides kits suitable for accomplishing these methods, which include a sealable container comprising a solution containing adenosine, an adenosine derivative or analog, an adenosine receptor agonist, a chemical entity that is taken up by a nucleoside transporter, or a chemical entity that binds to and/or modulates a nucleoside transporter, or an active compound, into which a biological sample may be placed and stored until further processed.

Methods of the present invention may also be used to preserve other non-living biological matter, including, e.g., cut flowers and food products, including both animal and plant-based food products. Such methods will extend the shelf-life of such products, as well as reduce spoilage and associated disease. In various embodiments, cut flowers and food products such as vegetables or fruits, and meats, are contacted with a solution comprising adenosine, an adenosine derivative or analog, an adenosine receptor agonist, a chemical entity that is taken up by a nucleoside transporter, or a chemical entity that binds to and/or modulates a nucleoside transporter, or an active compound. Certain products, such as cut flowers or vegetables with stems, such as asparagus and broccoli, may be stored so that their stems remain in contact with a solution comprising adenosine, an adenosine derivative or analog, an adenosine receptor agonist, a chemical entity that is taken up by a nucleoside transporter, or a chemical entity that binds to and/or modulates a nucleoside transporter, or an active compound.

The present invention similarly provides methods of extending the shelf-life and reducing spoilage of other food products subject to spoilage, such as, e.g., wine, beer, and cheeses. In various embodiments, these non-biological products are contacted with or exposed to adenosine, an adenosine derivative or analog, an adenosine receptor agonist, a chemical entity that is taken up by a nucleoside transporter, or a chemical entity that binds to and/or modulates a nucleoside transporter, or an active compound. In certain embodiments, the product contains an effective amount of adenosine, an adenosine derivative or analog, an adenosine receptor agonist, a chemical entity that is taken up by a nucleoside transporter, or a chemical entity that binds to and/or modulates a nucleoside transporter, or an active compound, wherein the adenosine, adenosine receptor agonist, chemical entity, or active compound is safe for human ingestion. In other embodiments, a compound may be exposed to or contacted with adenosine, an adenosine derivative or analog, an adenosine receptor agonist, a chemical entity that is taken up by a nucleoside transporter, or a chemical entity that binds to and/or modulates a nucleoside transporter, or an active compound prior to packaging or bottling. In one embodiment, a beer or wine is stored or bottled under a gas comprising an active compound. In certain embodiments, the product is contacted with adenosine, an adenosine derivative or analog, an adenosine receptor agonist, a chemical entity that is taken up by a nucleoside transporter, or a chemical entity that binds to and/or modulates a nucleoside transporter, or an active compound during a portion of the manufacturing process, e.g., while a wine is being stored in barrels.

Compositions and method of the present invention may also be used to preserve or increase the shelf-life of other products subject to spoilage or bacterial contamination, including, e.g., pharmaceutical products, health care products, and cosmetic products. These methods comprise contacting the product with adenosine, an adenosine derivative or analog, an adenosine receptor agonist, a chemical entity that is taken up by a nucleoside transporter, or a chemical entity that binds to and/or modulates a nucleoside transporter, or an active compound before packaging, or formulating the product in a solution comprising adenosine, an adenosine derivative or analog, an adenosine receptor agonist, a chemical entity that bind to and/or is taken up by a nucleoside transporter, or an active compound.

The amount of adenosine, adenosine derivative or analog, adenosine receptor agonist, chemical entity that bind to and/or is taken up by a nucleoside transporter, or active compound that is provided to non-living biological material or non-biological material can be about, at least, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 mg, mg/kg, or mg/m2, or any range derivable therein. Alternatively, the amount may be expressed as 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 mM or M, or any range derivable therein.

In various embodiments of the present invention, non-living biological material or non-biological material is exposed for about, at least, at least about, or at most about 30 seconds, 1, 2, 3, 4, 5, 10, 15, 20, 25, 30, 35, 40, 45, 50, 55 minutes, 1, 2, 3, 4, 5, 6, 7, 8, 9, 10, 11, 12, 13, 14, 15, 16, 17, 18, 19, 20, 21, 22, 23, 24 hours, 1, 2, 3, 4, 5, 6, 7 days or more, and any range or combination therein. In particular embodiments, exposure continues for the duration of the shelf-life of the material, and may be greater than one month, six months, one year, or even two years.

4. Combination Treatments

In certain embodiments, methods of the present invention comprise contacting biological material with a combination of adenosine, an adenosine derivative or analog, an adenosine receptor agonist, a chemical entity that is taken up by a nucleoside transporter, or a chemical entity that binds to and/or modulates a nucleoside transporter, and one or more additional active compounds. In other related embodiment, methods of the present invention comprise contacting biological material with adenosine, an adenosine derivative or analog, an adenosine receptor agonist, a chemical entity that is taken up by a nucleoside transporter, or a chemical entity that binds to and/or modulates a nucleoside transporter, in combination with another treatment that reduces metabolic activity. Examples of such other treatments include, but are not limited to, reduced oxygen conditions and reduced temperature conditions.

It is understood according to the present invention that the dosages of adenosine, adenosine derivative or analog, adenosine receptor agonist, chemical entity that bind to and/or is taken up by a nucleoside transporter, or other active compound, and duration of treatment are generally lower when using a combination of adenosine, an adenosine derivative or analog, an adenosine receptor agonist, a chemical entity that is taken up by a nucleoside transporter, or a chemical entity that binds to and/or modulates a nucleoside transporter, with another active compound or treatment as compared to using the adenosine, an adenosine derivative or analog, an adenosine receptor agonist, a chemical entity that is taken up by a nucleoside transporter, or a chemical entity that binds to and/or modulates a nucleoside transporter, active compound, or treatment alone. Accordingly, the combination treatments provided by the present invention may offer advantages associated with reduced side effects associated with treatment using certain active compounds or other treatments to reduce metabolic activity of prevent injury.

Methods of the present invention that involve providing to biological material adenosine, an adenosine derivative or analog, an adenosine receptor agonist, a chemical entity that is taken up by a nucleoside transporter, or a chemical entity that binds to and/or modulates a nucleoside transporter, in combination with one or more active compounds may be practiced, in various embodiments, by providing the adenosine or other chemical entity, and one or more additional active compounds simultaneously, contemporaneously, or at different times. For example, in one embodiment, biological material is contacted with adenosine, an adenosine derivative or analog, an adenosine receptor agonist, a chemical entity that is taken up by a nucleoside transporter, or a chemical entity that binds to and/or modulates a nucleoside transporter, and subsequently contacted with one or more additional active compounds. Alternatively, biological material is contacted with one or more additional active compound and then subsequently contacted with adenosine, an adenosine derivative or analog, an adenosine receptor agonist, a chemical entity that is taken up by a nucleoside transporter, or a chemical entity that binds to and/or modulates a nucleoside transporter. Contact with adenosine, an adenosine derivative or analog, an adenosine receptor agonist, a chemical entity that is taken up by a nucleoside transporter, or a chemical entity that binds to and/or modulates a nucleoside transporter, and one or more active compounds may be discrete, wherein contact with one is terminated prior to contact with another, or it may overlap or occur concurrently.

In the context of combination methods involving contacting biological material with adenosine and another treatment that reduces metabolic cellular or tissue damage due to injury or disease, the biological matter may be subjected to either adenosine or the other treatment at the same time, one before the other, or during overlapping time periods.

In other embodiments, the methods of the present invention include combination treatment with adenosine, an adenosine derivative or analog, an adenosine receptor agonist, a chemical entity that is taken up by a nucleoside transporter, or a chemical entity that binds to and/or modulates a nucleoside transporter, and another agents effective in the treatment of the specific disease or condition being treated (secondary therapy). For example, the treatment of stroke (antistroke treatment) typically involves an antiplatelet (aspirin, clopidogrel, dipyridamole, ticlopidine), an anticoagulant (heparin, warfarin), or a thrombolytic (tissue plasminogen activator). Any of these compounds may be used in combination with adenosine, an adenosine derivative or analog, an adenosine receptor agonist, a chemical entity that is taken up by a nucleoside transporter, or a chemical entity that binds to and/or modulates a nucleoside transporter, according to the methods of the present invention.

In other embodiments of the present invention, adenosine, an adenosine derivative or analog, an adenosine receptor agonist, a chemical entity that is taken up by a nucleoside transporter, or a chemical entity that binds to and/or modulates a nucleoside transporter, is provided to biological material in combination with an environmental condition associated with reduced metabolic activity of biological material. Such environmental conditions include low temperatures and hypoxic or anoxic conditions.

Standard methods of achieving hypoxia or anoxia are well established and include using environmental chambers that rely on chemical catalysts to remove oxygen from the chamber. Such chambers are available commercially from, for example, BD Diagnostic Systems (Sparks, MD) as GASPAK Disposable Hydrogen+Carbon Dioxide Envelopes or BIO-BAG Environmental Chambers. Alternatively, oxygen may be depleted by exchanging the air in a chamber with a non-oxygen gas, such as nitrogen. Oxygen concentration may be determined, for example using a FYRITE Oxygen Analyzer (Bacharach, Pittsburgh Pa.).

It is contemplated that methods of the invention can use a combination of exposure to adenosine, an adenosine derivative or analog, an adenosine receptor agonist, a chemical entity that is taken up by a nucleoside transporter, or a chemical entity that binds to and/or modulates a nucleoside transporter, and alteration of oxygen concentrations compared to room air. Moreover, the oxygen concentration of the environment containing biological matter can be about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74,75, 76, 77, 78, 79, 80, 81, 82, 83, 84, 85, 86, 87, 88, 89, 90, 91, 92, 93, 94, 95, 96, 97, 98, 99, or 100%, or any range derivable therein. Moreover, it is contemplated that a change in concentration can be any of the above percentages or ranges, in terms of a decrease or increase compared to room air or to a controlled environment.

In certain embodiments, reduced temperature conditions are sub-physiological temperatures with reference to the particular biological material being treated, which are understood to differ depending upon the biological material being treated. In particular embodiments, a biological matter is contacted with adenosine, an adenosine derivative or analog, an adenosine receptor agonist, a chemical entity that is taken up by a nucleoside transporter, or a chemical entity that binds to and/or modulates a nucleoside transporter, and also subjected to reduced temperature conditions of less than 37° C., les than 30° C., less than 25° C., less than 20° C., les than 15° C., less than 10° C., less than 5° C., less than 0° C., less than −20° C., or less than −70° C.

5. Pharmaceutical Administration and Compositions

The methods of the present invention comprise contacting a biological material with adenosine, an adenosine derivative or analog, an adenosine receptor agonist, a chemical entity that is taken up by a nucleoside transporter, or a chemical entity that binds to and/or modulates a nucleoside transporter, alone or in combination with one or more additional active compounds. The routes of administration of adenosine, an adenosine derivative or analog, an adenosine receptor agonist, a chemical entity that is taken up by a nucleoside transporter, or a chemical entity that binds to and/or modulates a nucleoside transporter, or other active compound will vary, naturally, with the location and nature of the condition to be treated, and include, e.g., inhalation, intradermal, transdermal, parenteral, intravenous, intramuscular, intranasal, subcutaneous, percutaneous, intratracheal, intraperitoneal, intratumoral, perfusion, lavage, direct injection, and oral administration and formulation. In particular embodiments, certain active compounds or chemical entities are administered topically, as solid dosage forms, using perfusion systems, or by catheter. In certain embodiments, adenosine and/or active compounds may be administered as medical gases by inhalation or intubation, as injectable liquids by intravascular, intravenous, intra-arterial, intracerobroventicular, intraperitoneal, or subcutaneous administration. In particular embodiments, the pharmaceutical compositions disclosed herein are administered parenterally, intradermally, intramuscularly, transdermally or even intraperitoneally, as described in U.S. Pat. Nos. 5,543,158; 5,641,515 and 5,399,363. In specific embodiments, adenosine is administered intraarterially, intravenously, or intraperitoneally. Gases may delivered as known in the art, including the use of a respiration system as described in U.S. Provisional Patent Applications 60/673,037 and 60/673,295 both filed on Apr. 20, 2005, as well as U.S. Provisional Patent Application 60/713,073, filed Aug. 31, 2005, and U.S. Provisional Patent Application 60/731,549, filed Oct. 28, 2005.

In addition, the amount of adenosine, an adenosine derivative or analog, an adenosine receptor agonist, a chemical entity that is taken up by a nucleoside transporter, or a chemical entity that binds to and/or modulates a nucleoside transporter, or other active compound provided to a biological material may vary depending on the type of biological material (cell type, tissue type, organism genus and species, etc.) and/or its size (weight, surface area, etc.). It will generally be the case that the larger the organism, the larger the dose. Therefore, an effective amount for a mouse will generally be lower than an effective amount for a rat, which will generally be lower than an effective amount for a dog, which will generally be lower than an effective amount for a human.

In certain embodiments, adenosine, an adenosine derivative or analog, an adenosine receptor agonist, a chemical entity that is taken up by a nucleoside transporter, or a chemical entity that binds to and/or modulates a nucleoside transporter, including 5′-AMP, is administered at a dosage of dosage of 0.1 μmol adenosine/g matter to 100 μmol adenosine/g biological material. In one embodiment, 5′-AMP is administered to a human at a dosage of 160-200 mg of AMP per day. In another embodiment, adenosine is provided to the biological material by infusion at a dosage in the range of 10 μg/kg/min to 350 μg/kg/min. In another embodiment, adenosine is administered to the biological material at a dosage in the range of 10 μg/kg/min to 100 μg/kg/min. In another embodiment, adenosine is administered to the biological material at a dosage in the range of 50 μg/kg/min to 70 μg/kg/min. In one embodiment, the infusion is continued for 30 minutes to 24 hours. In another embodiment the adenosine is provided to the material at a dosage in the range of 0.1 μmol adenosine/g material to 100 μmol adenosine/g material.

Similarly, the length of time of administration may vary depending on the type of biological material (cell type, tissue type, organism genus and species, etc.) and/or its size (weight, surface area, etc.) and will depend in part upon dosage form and route of administration. In particular embodiments, adenosine, an adenosine derivative or analog, an adenosine receptor agonist, a chemical entity that is taken up by a nucleoside transporter, or a chemical entity that binds to and/or modulates a nucleoside transporter, or an active compound is provided for about or at least 30 seconds, 1 minute, 2 minutes, 3 minutes, 5 minutes, 10 minutes, 15 minutes, 30 minutes, 1 hour, 2 hours, 3 hours, four hours five hours, six hours, eight hours, twelve hours, twenty-four hours, or greater than twenty-four hours. Adenosine, an adenosine derivative or analog, an adenosine receptor agonist, a chemical entity that is taken up by a nucleoside transporter, or a chemical entity that binds to and/or modulates a nucleoside transporter or an active compound may be administered in a single dose or multiple doses, with varying amounts of time between administered doses.

In the case of transplant, the present invention may be used pre-and or post-operatively to render host or graft materials quiescent. In a specific embodiment, a surgical site may be injected or perfused with a formulation comprising adenosine, an adenosine derivative or analog, an adenosine receptor agonist, a chemical entity that is taken up by a nucleoside transporter, or a chemical entity that binds to and/or modulates a nucleoside transporter. The perfusion may be continued post-surgery, for example, by leaving a catheter implanted at the site of the surgery.

Solutions of adenosine and/or other active compounds may be prepared in water or saline suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms. The pharmaceutical forms suitable for injectable use include sterile aqueous solutions or dispersions and sterile powders for the extemporaneous preparation of sterile injectable solutions or dispersions (U.S. Pat. No. 5,466,468). In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous, intratumoral and intraperitoneal administration. In this connection, sterile aqueous media that can be employed will be known to those of skill in the art in light of the present disclosure. For example, one dosage may be dissolved in 1 ml of isotonic NaCI solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

Sterile injectable solutions are prepared by incorporating the adenosine or active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

The phrase “pharmaceutically-acceptable” or “pharmacologically-acceptable” refers to molecular entities and compositions that do not produce an allergic or similar untoward reaction when administered to a human.

6. Articles of Manufacture

The present invention further provides articles of manufacture and kits useful in practicing the methods of the present invention. In one embodiment, an article of manufacture comprises packaging material and, contained within the packaging material, adenosine, an adenosine derivative or analog, an adenosine receptor agonist, a chemical entity that is taken up by a nucleoside transporter, or a chemical entity that binds to and/or modulates a nucleoside transporter, wherein the packaging material comprises a label that indicates that the adenosine, an adenosine derivative or analog, an adenosine receptor agonist, a chemical entity that is taken up by a nucleoside transporter, or a chemical entity that binds to and/or modulates a nucleoside transporter, can be used for enhancing the survivability of an in vivo biological material. In particular embodiment, the adenosine is 5′-AMP. The article of manufacture may further comprising a pharmaceutically acceptable diluent. In particular embodiments, the adenosine, an adenosine derivative or analog, an adenosine receptor agonist, a chemical entity that is taken up by a nucleoside transporter, or a chemical entity that binds to and/or modulates a nucleoside transporter, is provided in a first sealed container and the pharmaceutically acceptable diluent is provided in a second sealed container.

In other related embodiments, the articles of manufacture and kits of the present invention further comprise one or more additional active compounds, for use in a method of the present invention that includes combination therapy with adenosine, an adenosine derivative or analog, an adenosine receptor agonist, a chemical entity that is taken up by a nucleoside transporter, or a chemical entity that binds to and/or modulates a nucleoside transporter, and one or more additional active compounds. Typically, the one or more additional active compounds are each provided in separate sealed containers. Instructions for administering the combination of adenosine, an adenosine derivative or analog, an adenosine receptor agonist, a chemical entity that is taken up by a nucleoside transporter, or a chemical entity that binds to and/or modulates a nucleoside transporter, and active compound(s) are also optionally provided.

### EXAMPLES

### Example 1

### Protection from Lethal Hypoxia using 5′-AMP

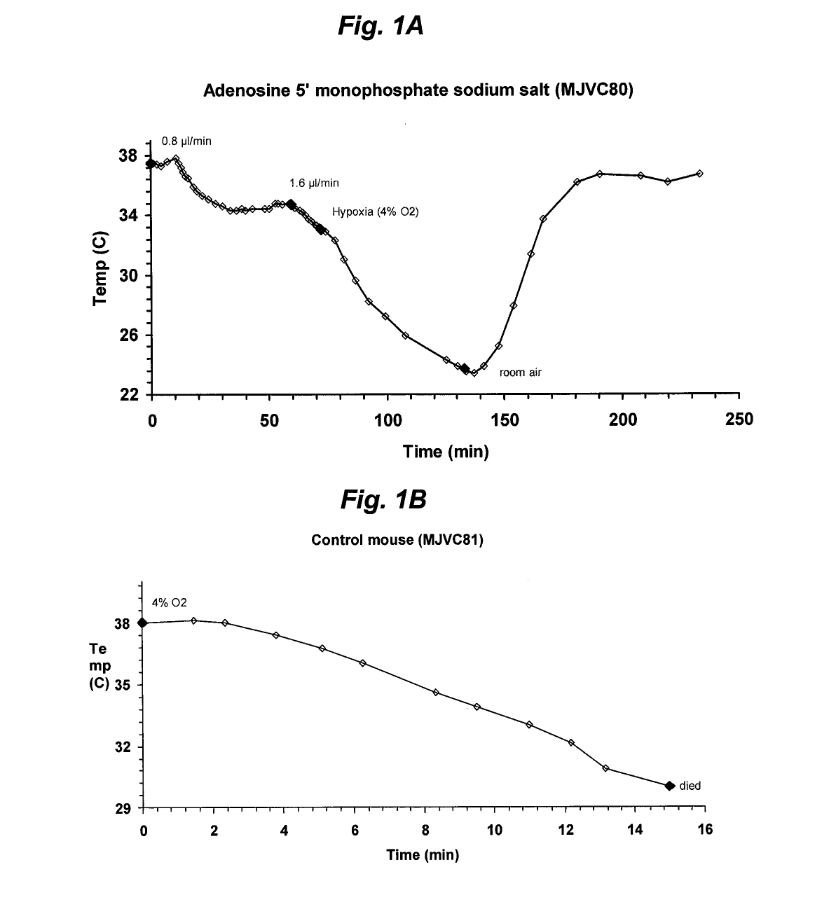

The ability of 5′-AMP to protect mice from lethal hypoxia was determined by comparing the effects of hypoxia on mice treated with 5′-AMP to the effects of hypoxia on untreated mice. Male C57BI/6 mice, 5-6 weeks old, with implanted jugular vein catheter (Taconic) were implanted dorsally with a subcutaneous RFID temperature sensor (IPTT-300, Bio Medic Data Systems, Inc.) and allowed to recover for at least 24 hours. Adenosine-5′-monophosphate (AMP sodium salt, Sigma) was dissolved in water, the osmolarity was adjusted with sodium chloride to 300 mOsm, the pH was adjusted with NaOH to 6.3, and the solution was filtered through a 0.2 um filter. The final AMP concentration was 40 mM. Mice were placed into a tall glass-bottom jar with opaque walls and infused via the jugular vein at a rate of 0.8 ul/min for 59.5 min followed by 1.6 ul/min for 12.8 min (total AMP dose=145 umol/kg) with a syringe pump (Harvard Apparatus), until their subcutaneous temperature had decreased to about 33° C. The infusion was stopped, and the mice were exposed for 60 min to an atmosphere of 4% oxygen constructed by mixing air and nitrogen using mass flow controllers (Sierra Instruments). During the infusion and exposure to hypoxia, the animals' subcutaneous temperature was read every 3-5 min and their vital signs were monitored. At the end if the hypoxic period, the animals were removed to ambient air. Temperature measurements were continued until the animals' temperature had returned to the normal range (FIGS. 1A and 1B). A control mouse was exposed to hypoxia, and its time of death was recorded.

The AMP-infused mouse (MJVC80) survived exposure to hypoxia for 60 min and recovered fully from the lowest temperature of 23.4° C. within 1 hour. The control mouse (MJVC81) died after 15 min of hypoxia with a final temperature of 30° C.

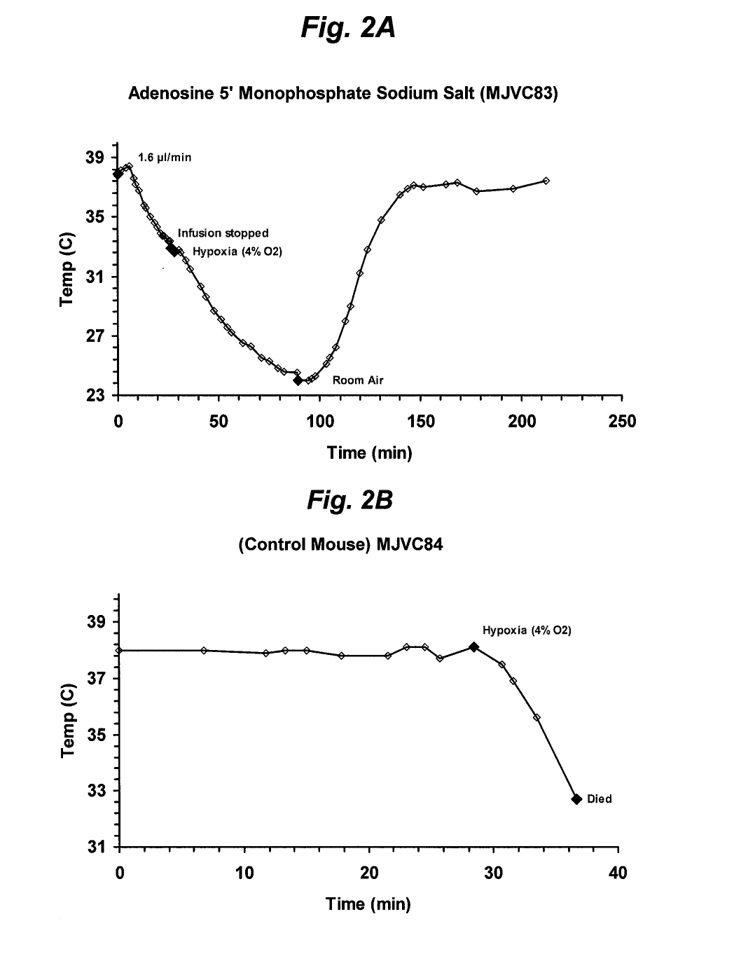

The same procedure described above was repeated under conditions wherein the mouse treated with 5′AMP received a constant infusion of 40 mM AMP at 1.6 ul/min for 26.5 min (total AMP dose of 69 umol/kg) until its subcutaneous temperature had reached 33 C. In this case, the AMP-infused mouse (MJVC83) survived exposure to hypoxia for 60 min and recovered fully from the lowest temperature of 24° C. within 1 hour. The control mouse (MJVC84) died after 8.5 min of hypoxia with a final temperature of 32.7° C. (FIGS. 2A and 2B).

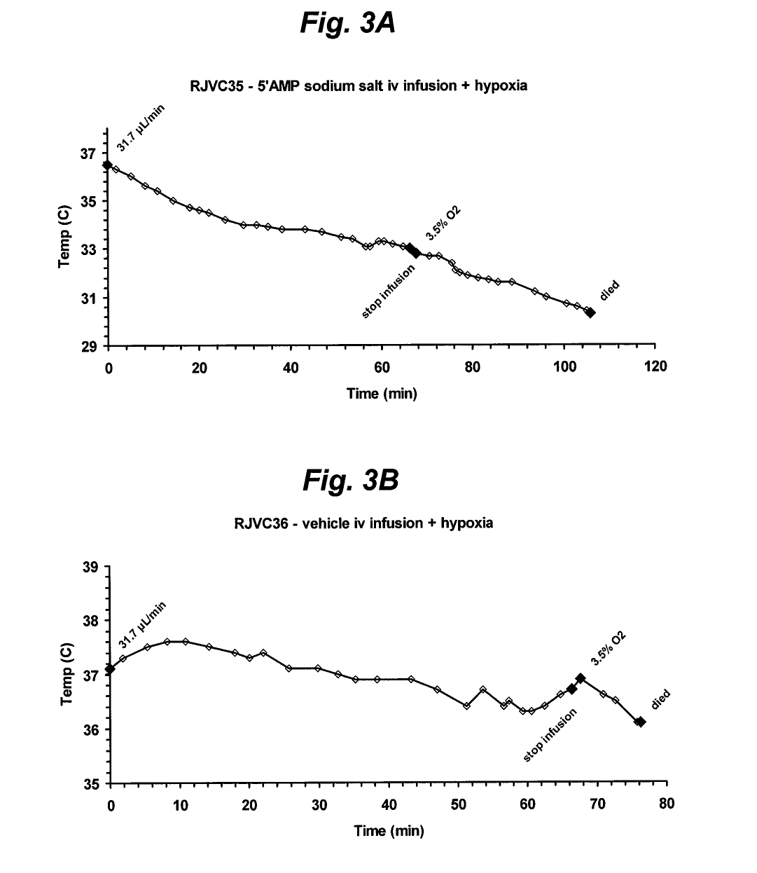

The procedure described above was also performed on Sprague Dawley rats (345-390 g) with jugular vein catheters. 40 mM 5′AMP of pH 6.4 was administered as a bolus (258 ul) followed by constant infusion at 31.7 ul/min for 66.4 min (total AMP dose 270 uM/kg). A control rat was infused with an equivalent volume of saline instead of 40 mM AMP. The hypoxic atmosphere contained 3.5% oxygen. Under these conditions, 5′AMP infusion extended the time of survival in 3.5% oxygen from 8.6 min (control rat RJVC36) to 38.2 min (RJVC35; FIGS. 3A and 3B).

Together, the results of these experiments establish that treatment with adenosine, e.g., 5′-AMP, protects animals from lethal hypoxia, either by preventing death or extending survival. In addition, these experiments support the conclusion that adenosine is useful in treating diseases caused by ischemia and hypoxia, such as, e.g., myocardial infarction, stroke, hemorrhagic shock, traumatic brain injury, kidney ischemia, tissue hypoperfusion, and trauma.

### Example 2

### Protection from Lethal Hypoxia using Adenosine

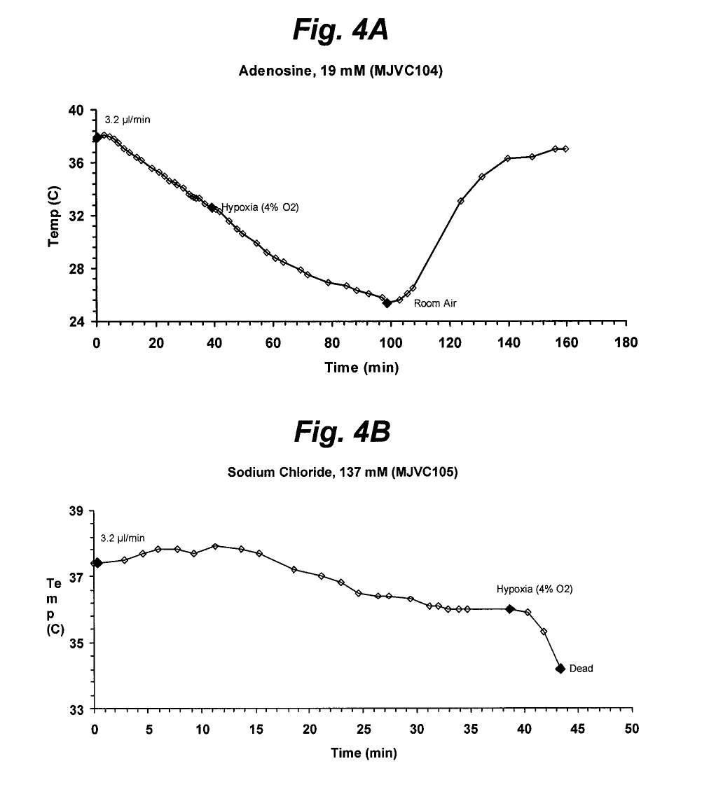

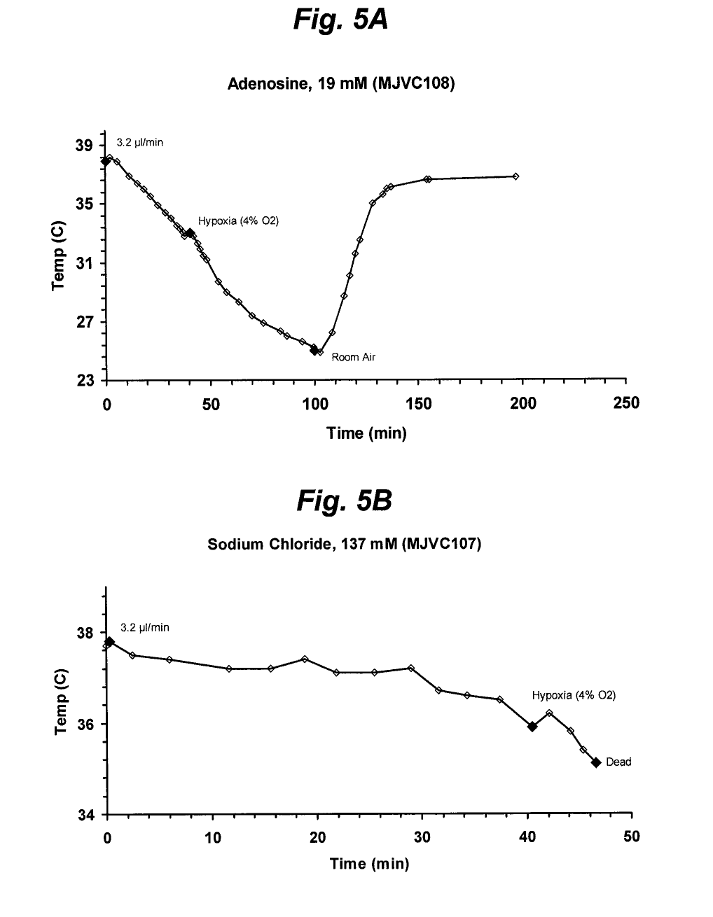

The ability of adenosine to protect mice from lethal hypoxia was determined by comparing the effects of hypoxia on mice treated with adenosine to the effects of hypoxia on untreated mice. Male C57BI/6 mice, 5-8 weeks old, with implanted jugular vein catheter (Taconic) were implanted dorsally with a subcutaneous RFID temperature sensor (PTT-300, Bio Medic Data Systems, Inc.) and allowed to recover for at least 24 hours after implantation. Mice were injected with adenosine (19 mM, 3.2 μl/min total dose approximately 27 mg/kg) and temperature was monitored via the temperature sensor until subcutaneous body temperature had decreased to about 33° C. The infusion was stopped, and these mice, as well as control mice injected with sodium chloride) were exposed for 60 min to an atmosphere of 4% oxygen constructed by mixing air and nitrogen using mass flow controllers (Sierra Instruments). During the infusion and exposure to hypoxia, the animals' subcutaneous temperature was monitored every 3-5 min and their vital signs were monitored. In parallel, a control mouse was injected with vehicle (sodium chloride; NaCI; 137 mM, 3.2 μl/min, total dose of NaCl administered was approximately 41 mg/kg) and put into a hypoxic atmosphere (4% O₂) at the same time as the mouse injected with adenosine, and its behavior and body temperature monitored. Following 60 min in hypoxic conditions, the mice (MJVC104 and MJVC 108) treated with adenosine were transferred into room air, and recovery of the mice was recorded by observing behavior and monitoring body temperature. The two mice injected with adenosine survived the hypoxic atmosphere (4% O₂) for 60 minutes and recovered completely after the return to room air (FIGS. 4A and 5A). No behavioral abnormalities were noted in these mice the following day. The control mice (MJVC105 and MJVC107) died after approximately six minutes in the hypoxic atmosphere (4% O₂; FIGS. 4B and 5B).

The results of these experiments establish that treatment with adenosine protects animals from lethal hypoxia, either by preventing death or extending survival. In addition, these experiments support the conclusion that adenosine is useful in treating diseases caused by ischemia and hypoxia, such as, e.g., myocardial infarction, stroke, hemorrhagic shock, traumatic brain injury, kidney ischemia, tissue hypoperfusion, and trauma.

All of the U.S. patents, U.S. patent application publications, U.S. patent applications, foreign patents, foreign patent applications and non-patent publications referred to in this specification and/or listed in the Application Data Sheet, are incorporated herein by reference, in their entirety.

From the foregoing it will be appreciated that, although specific embodiments of the invention have been described herein for purposes of illustration, various modifications may be made without deviating from the spirit and scope of the invention. Accordingly, the invention is not limited except as by the appended claims.

### REFERENCES

The following references, to the extent that they provide exemplary procedural or other details supplementary to those set forth herein, are specifically incorporated herein by reference.
U.S. Pat. No. 3,777,507
U.S. Pat. No. 3,881,990
U.S. Pat. No. 3,989,816
U.S. Pat. No. 3,995,444
U.S. Pat. No. 4,034,753
U.S. Pat. No. 4,186,565
U.S. Pat. No. 4,266,573
U.S. Pat. No. 4,292,817
U.S. Pat. No. 4,442,856
U.S. Pat. No. 4,444,762
U.S. Pat. No. 4,447,415
U.S. Pat. No. 4,473,637
U.S. Pat. No. 4,502,295
U.S. Pat. No. 4,559,258
U.S. Pat. No. 4,723,974
U.S. Pat. No. 4,745,759
U.S. Pat. No. 4,798,824
U.S. Pat. No. 4,828,976
U.S. Pat. No. 4,938,961
U.S. Pat. No. 4,951,482
U.S. Pat. No. 5,066,578
U.S. Pat. No. 5,157,930
U.S. Pat. No. 5,217,860
U.S. Pat. No. 5,231,025
U.S. Pat. No. 5,285,657
U.S. Pat. No. 5,326,706
U.S. Pat. No. 5,370,989
U.S. Pat. No. 5,395,314
U.S. Pat. No. 5,399,363
U.S. Pat. No. 5,405,742
U.S. Pat. No. 5,434,045
U.S. Pat. No. 5,466,468
U.S. Pat. No. 5,470,738
U.S. Pat. No. 5,476,763
U.S. Pat. No. 5,543,158
U.S. Pat. No. 5,552,267
U.S. Pat. No. 5,568,910
U.S. Pat. No. 5,569,579
U.S. Pat. No. 5,580,781
U.S. Pat. No. 5,599,659
U.S. Pat. No. 5,636,643
U.S. Pat. No. 5,641,515
U.S. Pat. No. 5,645,081
U.S. Pat. No. 5,693,462
U.S. Pat. No. 5,699,793
U.S. Pat. No. 5,719,174
U.S. Pat. No. 5,736,397
U.S. Pat. No. 5,739,169
U.S. Pat. No. 5,752,929
U.S. Pat. No. 5,801,005
U.S. Pat. No. 5,830,880
U.S. Pat. No. 5,846,945
U.S. Pat. No. 5,912,019
U.S. Pat. No. 5,952,168
U.S. Pat. No. 6,013,256
U.S. Pat. No. 6,046,046
U.S. Pat. No. 6,054,261
U.S. Pat. No. 6,057,148
U.S. Pat. No. 6,100,082
U.S. Pat. No. 6,187,529
U.S. Pat. No. 6,365,338
U.S. Pat. No. 6,490,880
U.S. Pat. No. 6,492,103
U.S. Pat. No. 6,524,785
U.S. Pat. No. 6,552,083
U.S. Pat. No. 6,602,277
U.S. Pat. No. 6,790,603
U.S. patent application Ser. No. 10/971,575,
U.S. patent application Ser. No. 10/971,576
U.S. patent application Ser. No. 10/972,063
U.S. Prov. Appln. 60/513,458
U.S. Prov. Appln. 60/548,150
U.S. Prov. Appln. 60/557,942
U.S. Prov. Appln. 60/673,037
U.S. Prov. Appln. 60/673,295
U.S. Prov. Appln. 60/713,073
U.S. Prov. Appln. 60/731,549
U.S. Prov. Appln. 60/762462
Alam, Antioxid Redox Signal, 4(4):559-62, 2002.
Amersi et al., ]Hepatology, 35(4):815-823, 2002.
Austin-Ward and Villaseca, Revista Medica de Chile, 126(7):838-845, 1998.
Baldwin S A et al., Pflugers Arch. 447(5):735-43, 2004 February, Epub 28 Jun. 2003.
Baldwin, et al., Molecular Medicine Today 5, 216-224, 1999.
Barton & Ollis, Oxford, UK, Jones (Ed.), Pergamon Press, 3:373-487, 1979.
Baskin and Wang, Tetrahedron Lett., 43:8479-8483, 2002.
Baskin et al., Org. Lett., 4:4423, 2002.
Beauchamp et al., Crit. Rev. Toxicol. 13, 25, 1984.
Beck et al., Proc. Soc. Exp. Biol. Med. 86, 823, 1954.
Behringer et al., Crit Care Med., 31(5):1523-1531, 2003.
Bellamy et al., Crit. Care Med., 24(2 Suppl):S24-47, 1996.
Bernard et al., J. Thorac. Cardiovasc. Surg. 90:235-242, 1985.
Bernard et al., N. Engl. J. Med., 346(8):557-563, 2002.
Blackstone et al., Science, 308:518, 2005.
Boyce and Ham, J. Invest. Dermatol., 81:335-405, 1983.
Boyce and Ham, J. Tissue Culture Methods, 9:83-93, 1985.
Briese, Neurosci. Biobehav. Rev., 22(3):427-436, 1998.
Brizel, Seminars Radiation Oncol., 8(4Suppl): 17-20, 1998.
Brouard et al., J. Biol. Chem., 277(20):17950-17961, 2002.
Bukowski et al., Clinical Cancer Res., 4(10):2337-2347, 1998.
Burns & Murphey, Arch. Biochem. Biophys., 339:33-39, 1997. 1997
Burns et al., Arch. Biochem. Bipophys, 10:60-68, 1995.
Cairns et al., J. Am. Chem. Soc., 74:3982, 1952.
Carter, et al., Trends in Parasitolology 17, 142-145, 2001.
Cerqueira M D, Am J Cardiol. 94(2A):33D-40D; 22 Jul. 2004, discussion 40D-42D
Chapter IV; Chapter VI; Chapter VII; Chapter VIII; Chapter IX of Klayman, D. L.; Gunther, W. H. H. Eds, Wiley Interscience, New York, 1973.
Chasteen and Bentley, Chem. Rev., 103(1):1-25, 2003.
Christodoulides et al., Microbiology, 144(Pt 11):3027-3037, 1998.
CIIT (Chemical Industry Institute of Toxicology), In: 90 day vaporinhalation toxicity study of hydrogen sulfide, Toxigenics, 420-0710, 1983.
Clive et al., J. Org. Chem., 47:1641, 1982.
Cloarec & Charette, Org. Lett., 6:4731, 2004.
Cohen et al., Ann. Thorac. Surg., 67(5):1489-1491 , 1999.
Cristalli G et al., Curr Top Med Chem. 3(4):387-401, 2003.
Curran, Seminars Radiation Oncol., 8(4Suppl):2-4, 1998.
Dalpiaz A et al., Curr Med Chem. 9(21):1923-37, 2002 November.
Davidson et al., J. Immunother., 21(5):389-398, 1998.
Davis (1994)
Demuynck and Vialle, Bulletin de la Societe Chimique de France, 4:1213-1218, 1967
Demuynck et al., Bulletin de la Societe Chimique de France, 3366-3367, 1966.
Demuynck et al., Bulletin de la Societe Chimique de France, 8:2748-2754, 1967.
Dhanasekaran et al., J. Biol. Chem., 279:37575-37587, 2004.
Dillman, Cancer Biother. Radiopharm., 14(1):5-10, 1999.
Dittmer and Hoey, In: The Chemistry of Sulphinic Acids, Esters, and Their Derivatives, Wiley: Chichester, U.K., 239-273, 1990.
Dorman et al. Neurotoxicol. Teratol., 22(1):71-84, 2000.
Dulak et al., Antioxid. Redox Signal, 4(2):229-240, 2002.
Duus,. In Comprehensive Organic Chemistry: The Synthesis and Reactions of Organic Compounds, 1^{st} Ed., 1994.
Eto et al., Biochem. Biphys. Res. Commun., 293:1483-1488, 2002.
Ganther, Carcinogenesis 20(9): 1657-66 (1999)
Gao Z G et al. Mini Rev Med Chem. 5(6):545-53, 2005.
Gao Z G et al., Curr Top Med Chem. 4(8):855-62, 2004.
Gilbert et al., LANCET, 355:375-376, 2000.
Gladysz et al., J. Org. Chem., 43:1204, 1987.
Glass, Phosph. Sulfur Silicon Rel. Elem., 136, 137, 138:159-174, 1998.
Gorman et al., Toxicology, 187(1):25-38, 2003.
Gray J H et al., Pflugers Arch. 447(5):728-34, 2004 February, Epub 11 Jul. 2003.
Griffiths, et al., Nature Medicine 3, 89-93, 1997.
Guillemin et al., Cell, 89(1):9-12, 1997.
Hanibuchi et al., Intl. J. Cancer, 78(4):480-45, 1998.
Hannan et al., JAMA, 290(6):773-780, 2003.
Harris, J. Org. Chem., 25:225, 1960.
Harris, J. Org. Chem., 30:2190, 1965.
Hays, In: Studies of the Effects of Atmospheric Hydrogen Sulfide in Animals, thesis dissertation, University of Missouri-Columbia, 1972.
Headrick J P et al., Vascul Pharmacol. 42(5-6):271-9, 2005 April-May, Epub 19 Apr. 2005.
Hellstrand et al., Acta Oncologica, 37(4):347-353, 1998.
Higuchi and Fukamachi, Folia Pharmacologica Japonica, 73(3):307-319, 1977.
Hobert et al., Organomet allics,20: 1370, 2001.
Hochachka et al., Comp. Biochem. Physiol. B Biochem. Mol. Biol., 130(4):435-459, 2001.
Hochachka et al., Proc. Natl. Acad. Sci. USA, 93(18):9493-94938, 1996.
Hui and Hashimoto, Infection Immun., 66(11):5329-5336, 1998.
Hutchinson S A et al., Curr Pharm Des. 10(17):2021-39, 2004.
Hwang & Greenberg, Biochemistry, 38:14248, 1999.
Hyde, et al., Molecular Membrane Biology 18, 53-63, 2001.
Hyspler et al., J. Chromatography, 770:255-259, 2002.
Innicenti et al., Bioorg. Med. Chem. Lett. 14, 5769 (2004).
Jacobson K A et al., Nat Rev Drug Discov. 5(3):247-64, 2006 March.
Jiang et al., Am. J. Physiol. Cell Physiol., 280:1140-1150, 2001.
Ju et al., J. Neuropathol. Exp. Neurol., 59(3):241-50, 2000.
Kamoun, Amino Acids 26, 243, 2004.
Kelso et al., J. Biol. Chem., 276:4588-4596, 2001.
Khan et al., Toxicol. Applied Pharmacol., 103:482-490, 1990.
Kilburn and Warshaw, Toxicology Indust. Health, 11 (2): 185-197, 1995.
Kilburn, Environ. Health, 54(3):150, 1999
Kilburn, Environ. Res., 81(2):92-99, 1999.
Knapp and Darout, Org. Lett., 7:203, 2005.
Kong W et al., Curr Drug Metab. 5(1):63-84, 2004 February.
Kontou et al., J. Agricultureal and Food Chem., 52:1212, 2004.
Kopecky et al., American Heart Journal 146:146-152, 2003.
Kuroda et al., Transplantation, 46(3):457-460, 1988.
Kuroda et al., Transplantation, 46(3):457-460, 1988.
Lai et al., Biochemistry, 40:4904-4910, 2001.
Langer et al., Biochemistry 33:14034, 1994.
Langer et al., Biochemistry, 33:10867, 1997.
Ledingham et al., Circulation, 82(2):IV351-358, 1990.
Ledingham et al., J. Thorac. Cardiobasc. Surg., 93:240-246, 1987.
Lin et al., Antimicrob Agents Chemother. 31(9): 1431-1433, 1987.
Liu et al., J. Org. Chem., 67:9267, 2002.
Lukashev D et al., Drug Discov Today 9(9):403-9, 1 May 2001.
Lundgren-Eriksson et al., Anticancer Res. 2001 September-October;21 (5):3269-74.
Mackey, et al., Drug Resistance Updates 1, 310-324, 1998.
Mehlhorn et al., Cardiovasc Surg., 9(5):482-486 , 2001.
Menasche et al., Eur J. Cardio. Thorax. Surg., 8:207-213, 1994.
Michaels et al., Circulation, 106(23):el 87-190, 2002.
Mugesh et al., Chem. Rev., 101:2125, 2001.
Muller C E, Curr Top Med Chem. 3(4):445-62, 2003.
Murai and Kato, In: Organoselenium Chemistry: Modern Developments in Organic Synthesis, Wirth (Ed.), Springer, N.Y., Vo. 28, 2000.
Murai, et al., J. Org. Chem., 66:8101, 2001.
Netherton & Fu, Org. Lett, 3:4295, 2001.
Noguchi et al., Biochemistry, 42:11642, 2003.
Nogueira et al., Chem. Rev., 104:6255, 2004.
Noji T et al., Eur J Pharmacol. 495(1):1-16, 8 Jul. 2004.
Nystul et al., Science, 302(5647):1038-1041, 2003.
O'Sullivan et al., J. Am. Chem. Soc., 126:2194, 2004.
Olojo et al., J. Phys. Chem. A, 108:1018, 2004.
Otterbein et al., Am. J. Physiol. Lung Cell Mol. Physiol., 279(6):L1029-L1037, 2000.
Otterbein et al., Trends Immunol., 24(8):449-455, 2003.
Padilla et al., Molec. Biology of the Cell, 13:1473-1483, 2002.
Padilla et al., Proc. Natl. Acad. Sci. USA, 98(13):7331-7335, 2001.
Partlo et al., Neurotoxicology, 22(2): 177-189, 2001.
PCT Appln. WO 94/17178
Petersen, Biochemica et Biophysica Acta, 460:299-307, 1977.
Pietras et al., Oncogene, 17(17):2235-2249, 1998.
Podgorska M et al., Acta Biochim Pol. 52(4):749-58, 2005, Epub 25 Oct. 2005.
Punch et al., 2001
Qin et al., Proc. Natl. Acad. Sci. USA, 95(24):14411-14416, 1998.
Quirante et al., J. Org. Chem., 67:2323, 2002.
Rager et al., NC Med. J., 65(1):18-25, 2004.
Reigan et al. J. Med. Chem., 48:392, 2005.
Remington's Pharmaceutical Sciences, 15^{th} ed., pages 1035-1038 and 1570-1580, Mack Publishing Company, Easton, Pa., 1980.
Rogers et al., Genome, 8:711-713, 1997.
Ryter and Otterbein, BioEssays, 26:270-280, 2004.
Seburg abd Squires, Intl. J. Mass Spectrometry Ion Proc., 167/168:541, 1997.
Semenza, Cell, 98(3):281-284, 1999.
Semenza, Trends Mol. Med., 7(8):345-350, 2001.
Shaw (1996)
Shawali et al., J. Org. Chem., 61:4055, 2001.
Shen et al., J. Agric. Food Chem., 50:2644, 2002.
Shryock, et al., American Journal of Cardiology 79(12A), 2-10, 1997.
Smith et al., Eur. J. Biochem., 263:709-716, 1999.
Soledad et al., Org. Lett., 3:1213, 2001.
Steudel, Chem. Rev., 102:3905, 2002.
Struve et al., Neurotoxicology, 22(3):375-385, 2001.
Sullivan G W, Curr Opin Investig Drugs 4(11):1313-9, 2003 November.
Sundarrajan et al., Macromolecules, 35:3331, 2002.
Supuran et al., Med. Res. Rev., 23(2):146-189, 2003.
Sweeney, In: A Survey of Compounds from the Antiradiation Drug Development Program of the U.S. Army Medical Research and Development Command. Walter Reed Army Institute of Research, Washington D.C., 1979.
Teodoro and OFarrell, EMBO J., 22(3):580-587, 2003.
The Hypothermia After Cardiac Arrest Study Group et al., 2002.
Thorn J A, et al., Gen Pharmacol. 27(4):613-20, 1996 June.
Tisherman, Crit. Care Med., 32(2):S46-S50, 2004.
Van Voorhies et al., J. Exp. Biol., 203(Pt 16):2467-2478, 2000.
Wang et al., 1992
Wang et al., 1993
Wang et al., 1994
Wang, FASEB J., 16(13): 1792-1798, 2002.
Yaffe et al., Crit. Care Med., 32(2):S51-55, 2004.
Yaghi et al., Nature, 423(6941):705-714, 2003.
Yan L et al., Expert Opin Emerg Drugs. 8(2):537-76, 2003 November.
Yang et al., J. Agric. Food Chem., 52:7051, 2004.
Yoshikawa et al., J. Biochem. (Tokyo), 71:859-872, 1972.
Zablocki J A et al., Curr Top Med Chem. 4(8):839-54, 2004.
Zhang et al., J. Appl. Physiol. 96(1):392-397, 2004.
Zhang et al., J. Org. Chem. 63:5314, 1998.
Zhang et al., Nature 439(19):340-343, 2006.
Ziegler, Ann. Rev. Biochem. 54, 305, 1985.

1. A method for enhancing the survivability of a biological material exposed to ischemic or hypoxic conditions comprising contacting the biological material with an effective amount of a chemical entity selected from the group consisting of:
(a) adenosine or a derivative, analog, or salt thereof;
(b) an adenosine receptor agonist;
(c) a chemical entity that is transported into a cell by a nucleoside transporter; and
(d) a chemical entity that binds to and/or modulates the action of a nucleoside transporter.

2. The method of claim 1, wherein the chemical entity is adenosine.

3. The method of claim 2, wherein the adenosine is 5′-AMP.

4. (canceled)

5. The method of claim 1, wherein the ischemic or hypoxic condition result from an injury to the material, the onset or progression of a disease that adversely affects the material, or hemorrhaging of the material.

6-10. (canceled)

11. The method of claim 1, wherein the biological material is selected from the group consisting of: cells, tissues, organs, organisms, and animals.

12. (canceled)

13. The method of claim 11, wherein the animal is a mammal.

14-15. (canceled)

16. The method of claim 11, wherein the biological material is to be transplanted.

17. The method of claim 1, wherein the biological material is at risk for reperfusion injury or hemorrhagic shock.

18-19. (canceled)

20. The method of claim 1, wherein the chemical entity is adenosine, which is provided to the biological material by infusion at a dosage in the range of 10 μg/kg/min to 350 μg/kg/min.

21-24. (canceled)

25. The method of claim 1, further comprising contacting the biological material with an effective amount of an active compound.

26-37. (canceled)

38. The method of claim 1, wherein the chemical entity is provided to the biological material as a pharmaceutical composition.

39. A method for preventing or reducing damage to a biological material exposed to ischemic or hypoxic conditions comprising contacting the biological material with an effective amount of a chemical entity selected from the group consisting of:
(a) adenosine or a derivative, analog, or salt thereof;
(b) an adenosine receptor agonist;
(c) a chemical entity that is transported into a cell by a nucleoside transporter; and
(d) a chemical entity that binds to and/or modulates the action of a nucleoside transporter.

40. A method for reversibly inhibiting metabolism in a biological material comprising contacting the biological material with an effective amount of a chemical entity selected from the group consisting of:
(a) adenosine or a derivative, analog, or salt thereof;
(b) an adenosine receptor agonist;
(c) a chemical entity that is transported into a cell by a nucleoside transporter; and
(d) a chemical entity that binds to and/or modulates the action of a nucleoside transporter.

41. (canceled)

42. A method of enhancing survivability of a mammal suffering from hemorrhagic shock or at risk of hemorrhagic shock, comprising contacting the mammal with an effective amount of a chemical entity selected from the group consisting of:
(a) adenosine or a derivative, analog, or salt thereof;
(b) an adenosine receptor agonist;
(c) a chemical entity that is transported into a cell by a nucleoside transporter; and
(d) a chemical entity that binds to and/or modulates the action of a nucleoside transporter.

43. (canceled)

44. A method of enhancing survivability of a mammal undergoing a surgery, comprising contacting the mammal with an effective amount of a chemical entity selected from the group consisting of:
(a) adenosine or a derivative, analog, or salt thereof;
(b) an adenosine receptor agonist;
(c) a chemical entity that is transported into a cell by a nucleoside transporter; and
(d) a chemical entity that binds to and/or modulates the action of a nucleoside transporter.

45. (canceled)

46. A method of preserving biological material ex vivo comprising contacting the biological material with a chemical entity selected from the group consisting of:
(a) adenosine or a derivative, analog, or salt thereof;
(b) an adenosine receptor agonist;
(c) a chemical entity that is transported into a cell by a nucleoside transporter; and
(d) a chemical entity that binds to and/or modulates the action of a nucleoside transporter.

47-59. (canceled)

60. A pharmaceutical composition comprising an active compound, a pharmaceutically acceptable diluent or carrier, and a chemical entity selected from the group consisting of:
(a) adenosine or a derivative, analog, or salt thereof;
(b) an adenosine receptor agonist;
(c) a chemical entity that is transported into a cell by a nucleoside transporter; and
(d) a chemical entity that binds to and/or modulates the action of a nucleoside transporter,
wherein said composition is formulated for administration to a mammal.

61-66. (canceled)
